(19) United States
(12) Patent Application Publication
(10) Pub. No.: US 2012/0260371 A1
(43) Pub. Date: Oct. 11, 2012

(54) Polypeptides Having Cellobiohydrolase Activity and Polynucleotides Encoding Same

(75) Inventors: Kimberly Brown, Elk Grove, CA (US); Eric Abbate, Vacaville, CA (US); Nikolaj Spodsberg, Bagsvaerd (DK)

(73) Assignees: NOVOZYMES A/S, Bagsvaerd (DK); NOVOZYMES, INC., Davis, CA (US)

(21) Appl. No.: 13/503,835

(22) PCT Filed: Oct. 28, 2010
(86) PCT No.: PCT/US10/54484

§ 371 Date: Jun. 26, 2012

Related U.S. Application Data
(60) Provisional application No. 61/256,074, filed on Oct. 29, 2009.

(51) Int. Cl. C12N 9/42; C12N 1/21; C12N 5/10; C12N 1/15; C12N 1/19; C12P 7/10; A01H 5/00; C07H 21/02; C12N 9/99; C12N 15/31; C12P 21/00; C12N 15/56; C12N 15/09

(52) U.S. Cl. 800/298; 435/209; 536/23.2; 435/252.31; 435/252.3; 435/252.35; 435/252.33; 435/252.34; 435/325; 435/348; 435/419; 435/254.11; 435/254.2; 435/254.22; 435/254.23; 435/254.21; 435/254.3; 435/254.7; 435/254.8; 435/254.4; 435/254.5; 435/254.6; 435/440; 536/24.5; 435/184; 536/23.74; 435/69.1; 435/165

(57) ABSTRACT

The present invention relates to isolated polypeptides having cellobiohydrolase activity and isolated polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### REFERENCE TO A DEPOSIT OF BIOLOGICAL MATERIAL

This application contains a reference to a deposit of biological material, which deposit is incorporated herein by reference.

### BACKGROUND OF THE INVENTION

1. Field of the Invention

The present invention relates to polypeptides having cellobiohydrolase activity and polynucleotides encoding the polypeptides. The invention also relates to nucleic acid constructs, vectors, and host cells comprising the polynucleotides as well as methods of producing and using the polypeptides.

2. Description of the Related Art

Cellulose is a polymer of the simple sugar glucose linked by beta-1,4 bonds. Many microorganisms produce enzymes that hydrolyze beta-linked glucans. These enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose from the ends of the cellulose polymer. Cellobiose is a water-soluble beta-1,4-linked dimer of glucose. Beta-glucosidases hydrolyze cellobiose to glucose.

The conversion of lignocellulosic feedstocks into ethanol has the advantages of the ready availability of large amounts of feedstock, the desirability of avoiding burning or land filling the materials, and the cleanliness of the ethanol fuel. Wood, agricultural residues, herbaceous crops, and municipal solid wastes have been considered as feedstocks for ethanol production. These materials primarily consist of cellulose, hemicellulose, and lignin. Once the cellulose is converted to glucose, the glucose is easily fermented by yeast into ethanol.

WO 2008/095033 discloses a fungal glycoside hydrolase.

It would be advantageous in the art to improve the ability to enzymatically degrade lignocellulosic feedstocks.

The present invention provides polypeptides having cellobiohydrolase activity and polynucleotides encoding the polypeptides.

### SUMMARY OF THE INVENTION

The present invention relates to isolated polypeptides having cellobiohydrolase activity selected from the group consisting of:

(a) a polypeptide comprising an amino acid sequence having at least 99% identity to the mature polypeptide of SEQ ID NO: 2;

(b) a polypeptide encoded by a polynucleotide comprising a nucleotide sequence having at least 99% identity to the mature polypeptide coding sequence of SEQ ID NO: 1; and

(d) a polypeptide comprising the mature polypeptide of SEQ ID NO: 2, or a fragment thereof having cellobiohydrolase activity.

The present invention also relates to isolated polynucleotides encoding the polypeptides of the present invention; nucleic acid constructs, recombinant expression vectors, and recombinant host cells comprising the polynucleotides; and methods of producing the polypeptides.

The present invention also relates to methods for degrading or converting a cellulosic material, comprising: treating the cellulosic material with an enzyme composition in the presence of a polypeptide of the present invention.

The present invention also relates to methods for producing a fermentation product, comprising: (a) saccharifying a cellulosic material with an enzyme composition in the presence of a polypeptide of the present invention; (b) fermenting the saccharified cellulosic material with one or more fermenting microorganisms to produce the fermentation product; and (c) recovering the fermentation product from the fermentation

The present invention also relates to methods of fermenting a cellulosic material, comprising: fermenting the cellulosic material with one or more fermenting microorganisms, wherein the cellulosic material is saccharified with an enzyme composition in the presence of a polypeptide of the present invention.

The present invention also relates to a polynucleotide encoding a signal peptide comprising or consisting of amino acids 1 to 18 of SEQ ID NO: 2, which is operably linked to a gene encoding a protein; nucleic acid constructs, expression vectors, and recombinant host cells comprising the polynucleotides; and methods of producing a protein.

### BRIEF DESCRIPTION OF THE FIGURES

FIGS. 1A and 1B show the cDNA sequence and the deduced amino acid sequence of an Aspergillus aculeatus strain NN000525 (IAM 2445) GH6 cellobiohydrolase gene (SEQ ID NOs: 1 and 2, respectively).

FIG. 2 shows the results of a 20% replacement (by protein) of a Trichoderma reesei cellulolytic protein preparation (loaded at 2 mg per g of cellulose) with A. aculeatus cellobiohydrolase in the hydrolysis of pretreated corn stover.

FIG. 3 shows a restriction map of pXYG1051-P6XY.

FIG. 4 shows a restriction map of pCR2.1-P6XY.

### DEFINITIONS

Cellobiohydrolase: The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178). For purposes of the present invention, cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581; and van Tilbeurgh et al., 1985, Eur. J. Biochem. 148: 329-334. The Lever et al. method can be employed to assess hydrolysis of cellulose in corn stover, while the methods of van Tilbeurgh et al. and Tomme et al. can be used to determine cellobiohydrolase I activity on 4-methylumbelliferyl-β-D-lactopyranoside.

Cellulolytic enzyme or cellulase: The term “cellulolytic enzyme” or “cellulase” means one or more (several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-20 mg of cellulolytic enzyme protein/g of cellulose in PCS for 3-7 days at 50° C. compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 72 hours, sugar analysis by AMINEX® HPX-87H column (Bio-Rad Laboratories, Inc., Hercules, Calif., USA).

Endoglucanase: The term “endoglucanase” means an endo-1,4-(1,3;1,4)-beta-D-glucan 4-glucanohydrolase (E.C. 3.2.1.4), which catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Beta-glucosidase: The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Polypeptide having cellulolytic enhancing activity: The term “polypeptide having cellulolytic enhancing activity” means a GH61 polypeptide that enhances the hydrolysis of a cellulosic material by enzyme having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in PCS, wherein total protein is comprised of 50-99.5% w/w cellulolytic enzyme protein and 0.5-50% w/w protein of a GH61 polypeptide having cellulolytic enhancing activity for 1-7 days at 50° C. compared to a control hydrolysis with equal total protein loading without cellulolytic enhancing activity (1-50 mg of cellulolytic protein/g of cellulose in PCS). In a preferred aspect, a mixture of CELLUCLAST® 1.5L (Novozymes A/S, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, more preferably at least 1.05-fold, more preferably at least 1.10-fold, more preferably at least 1.25-fold, more preferably at least 1.5-fold, more preferably at least 2-fold, more preferably at least 3-fold, more preferably at least 4-fold, more preferably at least 5-fold, even more preferably at least 10-fold, and most preferably at least 20-fold.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Hemicellulolytic enzyme or hemicellulase: The term “hemicellulolytic enzyme” or “hemicellulase” means one or more (several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetyxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families marked by numbers. Some families, with overall similar fold, can be further grouped into clans, marked alphabetically (e.g., GH-A). A most informative and updated classification of these and other carbohydrate active enzymes is available on the Carbohydrate-Active Enzymes (CAZy) database. Hemicellulolytic enzyme activities can be measured according to Ghose and Bisaria, 1987, Pure & Appl. Chem. 59: 1739-1752.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes is summarized in several publications including Biely and Puchard, Recent progress in the assays of xylanolytic enzymes, 2006, Journal of the Science of Food and Agriculture 86(11): 1636-1647; Spanikova and Biely, 2006, Glucuronoyl esterase—Novel carbohydrate esterase produced by Schizophyllum commune, FEBS Letters 580(19): 4597-4601; Herrmann, Vrsanska, Jurickova, Hirsch, Biely, and Kubicek, 1997, The beta-D-xylosidase of Trichoderma reesei is a multifunctional beta-D-xylan xylohydrolase, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

Beta-xylosidase: The term “beta-xylosidase” means a beta-D-xyloside xylohydrolase (E.C. 3.2.1.37) that catalyzes the exo-hydrolysis of short beta-(4)-xylooligosaccharides, to remove successive D-xylose residues from the non-reducing termini. For purposes of the present invention, one unit of beta-xylosidase is defined as 1.0 pmole of p-nitrophenolate anion produced per minute at 40° C., pH 5 from 1 mM p-nitrophenyl-beta-D-xyloside as substrate in 100 mM sodium citrate containing 0.01% TWEEN® 20.

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. For purposes of the present invention, acetylxylan esterase activity is determined using 0.5 mM p-nitrophenylacetate as substrate in 50 mM sodium acetate pH 5.0 containing 0.01% TWEEN™ 20. One unit of acetylxylan esterase is defined as the amount of enzyme capable of releasing 1 μmole of p-nitrophenolate anion per minute at pH 5, 25° C.

Feruloyl esterase: The term “feruloyl esterase” means a 4-hydroxy-3-methoxycinnamoyl-sugar hydrolase (EC 3.1.1.73) that catalyzes the hydrolysis of the 4-hydroxy-3-methoxycinnamoyl (feruloyl) group from an esterified sugar, which is usually arabinose in “natural” substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II. For purposes of the present invention, feruloyl esterase activity is determined using 0.5 mM p-nitrophenylferulate as substrate in 50 mM sodium acetate pH 5.0. One unit of feruloyl esterase equals the amount of enzyme capable of releasing 1 pmole of p-nitrophenolate anion per minute at pH 5, 25° C.

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. For purposes of the present invention, alpha-glucuronidase activity is determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, herbaceous material, agricultural residue, forestry residue, municipal solid waste, waste paper, and pulp and paper mill residue (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred aspect, the cellulosic material is lignocellulose, which comprises cellulose, hemicellulose, and lignin.

In one aspect, the cellulosic material is herbaceous material. In another aspect, the cellulosic material is agricultural residue. In another aspect, the cellulosic material is forestry residue. In another aspect, the cellulosic material is municipal solid waste. In another aspect, the cellulosic material is waste paper. In another aspect, the cellulosic material is pulp and paper mill residue.

In another aspect, the cellulosic material is corn stover. In another aspect, the cellulosic material is corn fiber. In another aspect, the cellulosic material is corn cob. In another aspect, the cellulosic material is orange peel. In another aspect, the cellulosic material is rice straw. In another aspect, the cellulosic material is wheat straw. In another aspect, the cellulosic material is switch grass. In another aspect, the cellulosic material is miscanthus. In another aspect, the cellulosic material is bagasse.

In another aspect, the cellulosic material is microcrystalline cellulose. In another aspect, the cellulosic material is bacterial cellulose. In another aspect, the cellulosic material is algal cellulose. In another aspect, the cellulosic material is cotton linter. In another aspect, the cellulosic material is amorphous phosphoric-acid treated cellulose. In another aspect, the cellulosic material is filter paper.

The cellulosic material may be used as is or may be subjected to pretreatment, using conventional methods known in the art, as described herein. In a preferred aspect, the cellulosic material is pretreated.

Pretreated corn stover: The term “PCS” or “Pretreated Corn Stover” means a cellulosic material derived from corn stover by treatment with heat and dilute sulfuric acid.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the methods of the present invention, any material containing xylan may be used. In a preferred aspect, the xylan-containing material is lignocellulose.

Isolated or Purified: The term “isolated” or “purified” means a polypeptide or polynucleotide that is removed from at least one component with which it is naturally associated. For example, a polypeptide may be at least 1% pure, e.g., at least 5% pure, at least 10% pure, at least 20% pure, at least 40% pure, at least 60% pure, at least 80% pure, at least 90% pure, or at least 95% pure, as determined by SDS-PAGE, and a polynucleotide may be at least 1% pure, e.g., at least 5% pure, at least 10% pure, at least 20% pure, at least 40% pure, at least 60% pure, at least 80% pure, at least 90% pure, or at least 95% pure, as determined by agarose electrophoresis.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 19 to 469 of SEQ ID NO: 2 based on the SignalP program (Nielsen et al., 1997, Protein Engineering 10:1-6) that predicts amino acids 1 to 18 of SEQ ID NO: 2 are a signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having cellobiohydrolase activity. In one aspect, the mature polypeptide coding sequence is nucleotides 55 to 1407 of SEQ ID NO: 1 based on the SignalP program (Nielsen et al., 1997, supra) that predicts nucleotides 1 to 54 of SEQ ID NO: 1 encode a signal peptide. In another aspect, the mature polypeptide coding sequence is the genomic DNA sequence of nucleotides 55 to 1407 of SEQ ID NO: 1.]

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Fragment: The term “fragment” means a polypeptide having one or more (several) amino acids deleted from the amino and/or carboxyl terminus of a mature polypeptide; wherein the fragment has cellobiohydrolase activity. In one aspect, a fragment contains at least 390 amino acid residues, e.g., at least 410 amino acid residues or at least 430 amino acid residues.

Subsequence: The term “subsequence” means a polynucleotide having one or more (several) nucleotides deleted from the 5′ and/or 3′ end of a mature polypeptide coding sequence; wherein the subsequence encodes a fragment having cellobiohydrolase activity. In one aspect, a subsequence contains at least 1170 nucleotides, e.g., at least 1230 nucleotides or at least 1290 nucleotides.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” means all components necessary for the expression of a polynucleotide encoding a polypeptide of the present invention. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Host cell: The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Variant: The term “variant” means a polypeptide having cellobiohydrolase activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding one or more (several) amino acids, e.g., 1-5 amino acids, adjacent to an amino acid occupying a position.

### DETAILED DESCRIPTION OF THE INVENTION

### Polypeptides Having Cellobiohydrolase Activity

The present invention relates to isolated polypeptides having cellobiohydrolase activity selected from the group consisting of:

(a) a polypeptide comprising an amino acid sequence having at least 99% identity to the mature polypeptide of SEQ ID NO: 2;

(b) a polypeptide encoded by a polynucleotide comprising a nucleotide sequence having at least 99% identity to the mature polypeptide coding sequence of SEQ ID NO: 1; and

(d) a polypeptide comprising the mature polypeptide of SEQ ID NO: 2, or a fragment thereof having cellobiohydrolase activity.

The present invention relates to isolated polypeptides having a sequence identity to the mature polypeptide of SEQ ID NO: 2 of at least 99%, e.g., 100%, which have cellobiohydrolase activity. In one aspect, the polypeptides differ by no more than ten amino acids, e.g., by five amino acids, by four amino acids, by three amino acids, by two amino acids, and by one amino acid from the mature polypeptide of SEQ ID NO: 2.

A polypeptide of the present invention preferably comprises or consists of the amino acid sequence of SEQ ID NO: 2 or an allelic variant thereof; or is a fragment thereof having cellobiohydrolase activity. In another aspect, the polypeptide comprises or consists of the mature polypeptide of SEQ ID NO: 2. In another preferred aspect, the polypeptide comprises or consists of amino acids 19 to 469 of SEQ ID NO: 2.

The present invention also relates to isolated polypeptides having cellobiohydrolase activity that are encoded by polynucleotides that hybridize under very high stringency conditions with (i) the mature polypeptide coding sequence of SEQ ID NO: 1, (ii) the genomic DNA sequence of the mature polypeptide coding sequence of SEQ ID NO: 1, or (iii) the full-length complementary strand of (i) or (ii) (J. Sambrook, E. F. Fritsch, and T. Maniatis, 1989, Molecular Cloning, A Laboratory Manual, 2d edition, Cold Spring Harbor, N.Y.).

The polynucleotide of SEQ ID NO: 1 or a subsequence thereof, as well as the amino acid sequence of SEQ ID NO: 2 or a fragment thereof, may be used to design nucleic acid probes to identify and clone DNA encoding polypeptides having cellobiohydrolase activity from strains of different genera or species according to methods well known in the art. In particular, such probes can be used for hybridization with the genomic DNA or cDNA of the genus or species of interest, following standard Southern blotting procedures, in order to identify and isolate the corresponding gene therein. Such probes can be considerably shorter than the entire sequence, but should be at least 14, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having cellobiohydrolase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1 or a subsequence thereof, the carrier material is preferably used in a Southern blot.

For purposes of the present invention, hybridization indicates that the polynucleotide hybridizes to a labeled nucleic acid probe corresponding to SEQ ID NO: 1; the mature polypeptide coding sequence of SEQ ID NO: 1; the genomic DNA sequence of the mature polypeptide coding sequence of SEQ ID NO: 1; its full-length complementary strand; or a subsequence thereof; under very low to very high stringency conditions. Molecules to which the nucleic acid probe hybridizes under these conditions can be detected using, for example, X-ray film.

In one aspect, the nucleic acid probe is the mature polypeptide coding sequence of SEQ ID NO: 1 or the genomic DNA sequence thereof. In another aspect, the nucleic acid probe is a polynucleotide that encodes the polypeptide of SEQ ID NO: 2 or the mature polypeptide thereof; or a fragment thereof. In another preferred aspect, the nucleic acid probe is SEQ ID NO: 1 or the genomic DNA sequence thereof. In another aspect, the nucleic acid probe is the polynucleotide contained in plasmid pCR2.1-P6XY which is contained in E. coli DSM 22994, wherein the polynucleotide encodes a polypeptide having cellobiohydrolase activity. In another aspect, the nucleic acid probe is the mature polypeptide coding region contained in plasmid pCR2.1-P6XY which is contained in E. coli DSM 22994.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), at 50° C. (low stringency), at 55° C. (medium stringency), at 60° C. (medium-high stringency), at 65° C. (high stringency), and at 70° C. (very high stringency).

For short probes of about 15 nucleotides to about 70 nucleotides in length, stringency conditions are defined as prehybridization and hybridization at about 5° C. to about 10° C. below the calculated Tₙ, using the calculation according to Bolton and McCarthy (1962, Proc. Natl. Acad. Sci. USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

The present invention also relates to isolated polypeptides having cellobiohydrolase activity encoded by polynucleotides having a sequence identity to the mature polypeptide coding sequence of SEQ ID NO: 1 or the genomic DNA sequence thereof of at least 99%, e.g., 100%.

The present invention also relates to variants comprising a substitution, deletion, and/or insertion of one or more (or several) amino acids of the mature polypeptide of SEQ ID NO: 2, or a homologous sequence thereof. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cellobiohydrolase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to the parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The total number of amino acid substitutions, deletions and/or insertions of the mature polypeptide of SEQ ID NO: 2 is not more than 10, e.g., 1, 2, 3, 4, 5, 6, 7, 8 or 9.

The polypeptide may be hybrid polypeptide in which a portion of one polypeptide is fused at the N-terminus or the C-terminus of a portion of another polypeptide.

The polypeptide may be a fused polypeptide or cleavable fusion polypeptide in which another polypeptide is fused at the N-terminus or the C-terminus of the polypeptide of the present invention. A fused polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Cellobiohydrolase Activity

A polypeptide having cellobiohydrolase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a gram-positive bacterial polypeptide such as a Bacillus, Clostridium, Enterococcus, Geobacillus, Lactobacillus, Lactococcus, Oceanobacillus, Staphylococcus, Streptococcus, or Streptomyces polypeptide having cellobiohydrolase activity, or a gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

The polypeptide may also be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

In another aspect, the polypeptide is an Aspergillus aculeatus polypeptide having cellobiohydrolase activity. In another aspect, the polypeptide is an Aspergillus aculeatus IAM 2445 polypeptide having cellobiohydrolase activity, e.g., the polypeptide comprising the mature polypeptide of SEQ ID NO: 2.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) using the above-mentioned probes. Techniques for isolating microorganisms from natural habitats are well known in the art. The polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are well known to those of ordinary skill in the art (see, e.g., Sambrook et al., 1989, supra).

### Polynucleotides

The present invention also relates to isolated polynucleotides encoding a polypeptide of the present invention.

The techniques used to isolate or clone a polynucleotide encoding a polypeptide are known in the art and include isolation from genomic DNA, preparation from cDNA, or a combination thereof. The cloning of the polynucleotides from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus, or a related organism and thus, for example, may be an allelic or species variant of the polypeptide encoding region of the polynucleotide.

The present invention also relates to isolated polynucleotides comprising or consisting of polynucleotides having a degree of sequence identity to the mature polypeptide coding sequence of SEQ ID NO: 1 or the genomic DNA sequence thereof of at least 99%, e.g., 100%, which encode a polypeptide having cellobiohydrolase activity.

Modification of a polynucleotide encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variant may be constructed on the basis of the polynucleotide presented as the mature polypeptide coding sequence of SEQ ID NO: 1 or the genomic DNA sequence thereof, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

The present invention also relates to isolated polynucleotides encoding polypeptides of the present invention, which hybridize under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the genomic DNA sequence of the mature polypeptide coding sequence of SEQ ID NO: 1, or (iii) the full-length complementary strand of (i) or (ii); or allelic variants and subsequences thereof (Sambrook et al., 1989, supra), as defined herein.

In one aspect, the polynucleotide comprises or consists of SEQ ID NO: 1, the mature polypeptide coding sequence of SEQ ID NO: 1, or the sequence contained in plasmid pCR2.1-P6XY which is contained in E. coli DSM 22994, or a subsequence of SEQ ID NO: 1 that encodes a fragment of SEQ ID NO: 2 having cellobiohydrolase activity, such as the polynucleotide of nucleotides 55 to 1407 of SEQ ID NO: 1.

### Nucleic Acid Constructs

The present invention also relates to nucleic acid constructs comprising a polynucleotide of the present invention operably linked to one or more (several) control sequences that direct the expression of the coding sequence in a suitable host cell under conditions compatible with the control sequences.

A polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, a polynucleotide that is recognized by a host cell for expression of a polynucleotide encoding a polypeptide of the present invention. The promoter sequence contains transcriptional control sequences that mediate the expression of the polypeptide. The promoter may be any polynucleotide that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, Streptomyces coelicolor agarase gene (dagA), and prokaryotic beta-lactamase gene (VIIIa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from a gene encoding a neutral alpha-amylase in Aspergilli in which the untranslated leader has been replaced by an untranslated leader from a gene encoding triose phosphate isomerase in Aspergilli; non-limiting examples include modified promoters from the gene encoding neutral alpha-amylase in Aspergillus niger in which the untranslated leader has been replaced by an untranslated leader from the gene encoding triose phosphate isomerase in Aspergillus nidulans or Aspergillus oryzae); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the host cell of choice may be used in the present invention.

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the host cell of choice may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present at the N-terminus of a polypeptide, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more (several) convenient restriction sites to allow for insertion or substitution of the polynucleotide encoding the polypeptide at such sites. Alternatively, the polynucleotide may be expressed by inserting the polynucleotide or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers that confer antibiotic resistance such as ampicillin, chloramphenicol, kanamycin, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more (several) control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any gram-positive or gram-negative bacterium. Gram-positive bacteria include, but not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may, for instance, be effected by protoplast transformation (see, e.g., Chang and Cohen, 1979, Mol. Gen. Genet. 168: 111-115), by using competent cells (see, e.g., Young and Spizizen, 1961, J. Bacteriol. 81: 823-829, or Dubnau and Davidoff-Abelson, 1971, J. Mol. Biol. 56: 209-221), by electroporation (see, e.g., Shigekawa and Dower, 1988, Biotechniques 6: 742-751), or by conjugation (see, e.g., Koehler and Thorne, 1987, J. Bacteriol. 169: 5271-5278). The introduction of DNA into an E. coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios 68: 189-207), by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising: (a) cultivating a cell, which in its wild-type form produces the polypeptide, under conditions conducive for production of the polypeptide; and (b) recovering the polypeptide. In a preferred aspect, the cell is of the genus Aspergillus. In a more preferred aspect, the cell is Aspergillus aculeatus. In a most preferred aspect, the cell is Aspergillus aculeatus IAM 2445.

The present invention also relates to methods of producing a polypeptide of the present invention, comprising: (a) cultivating a recombinant host cell of the present invention under conditions conducive for production of the polypeptide; and (b) recovering the polypeptide.

The host cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Plants

The present invention also relates to isolated plants, e.g., a transgenic plant, plant part, or plant cell, comprising an isolated polynucleotide of the present invention so as to express and produce the polypeptide in recoverable quantities. The polypeptide may be recovered from the plant or plant part. Alternatively, the plant or plant part containing the polypeptide may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seeds coats.

Also included within the scope of the present invention are the progeny of such plants, plant parts, and plant cells.

The transgenic plant or plant cell expressing a polypeptide may be constructed in accordance with methods known in the art. In short, the plant or plant cell is constructed by incorporating one or more (several) expression constructs encoding a polypeptide into the plant host genome or chloroplast genome and propagating the resulting modified plant or plant cell into a transgenic plant or plant cell.

The expression construct is conveniently a nucleic acid construct that comprises a polynucleotide encoding a polypeptide operably linked with appropriate regulatory sequences required for expression of the polynucleotide in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the polypeptide is desired to be expressed. For instance, the expression of the gene encoding a polypeptide may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, and the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may inducible by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a polypeptide in the plant. For instance, the promoter enhancer element may be an intron that is placed between the promoter and the polynucleotide encoding a polypeptide. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Mol. Biol. 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods for use in accordance with the present disclosure include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct prepared according to the present invention, transgenic plants may be made by crossing a plant having the construct to a second plant lacking the construct. For example, a construct encoding a polypeptide can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention, or a portion of a DNA construct prepared in accordance with the present invention. Crossing results in the introduction of a transgene into a plant line by cross pollinating a starting line with a donor plant line. Non-limiting examples of such steps are further articulated in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a polypeptide of the present invention comprising: (a) cultivating a transgenic plant or a plant cell comprising a polynucleotide encoding the polypeptide under conditions conducive for production of the polypeptide; and (b) recovering the polypeptide.

### Removal or Reduction of Cellobiohydrolase Activity

The present invention also relates to methods of producing a mutant of a parent cell, which comprises disrupting or deleting a polynucleotide, or a portion thereof, encoding a polypeptide of the present invention, which results in the mutant cell producing less of the polypeptide than the parent cell when cultivated under the same conditions.

The mutant cell may be constructed by reducing or eliminating expression of the polynucleotide using methods well known in the art, for example, insertions, disruptions, replacements, or deletions. In a preferred aspect, the polynucleotide is inactivated. The polynucleotide to be modified or inactivated may be, for example, the coding region or a part thereof essential for activity, or a regulatory element required for the expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the polynucleotide may be accomplished by introduction, substitution, or removal of one or more (several) nucleotides in the gene or a regulatory element required for the transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In a particularly preferred aspect, the polynucleotide is disrupted with a selectable marker such as those described herein.

The present invention also relates to methods of inhibiting the expression of a polypeptide having cellobiohydrolase activity in a cell, comprising administering to the cell or expressing in the cell a double-stranded RNA (dsRNA) molecule, wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length.

The dsRNA is preferably a small interfering RNA (sRNA) or a micro RNA (miRNA). In a preferred aspect, the dsRNA is small interfering RNA (siRNAs) for inhibiting transcription. In another preferred aspect, the dsRNA is micro RNA (miRNAs) for inhibiting translation.

The present invention also relates to such double-stranded RNA (dsRNA) molecules, comprising a portion of the mature polypeptide coding sequence of SEQ ID NO: 1 for inhibiting expression of the polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi).

The dsRNAs of the present invention can be used in gene-silencing. In one aspect, the invention provides methods to selectively degrade RNA using a dsRNAi of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824; and 6,515,109.

The present invention further relates to a mutant cell of a parent cell that comprises a disruption or deletion of a polynucleotide encoding the polypeptide or a control sequence thereof or a silenced gene encoding the polypeptide, which results in the mutant cell producing less of the polypeptide or no polypeptide compared to the parent cell.

The polypeptide-deficient mutant cells are particularly useful as host cells for the expression of native and heterologous polypeptides. Therefore, the present invention further relates to methods of producing a native or heterologous polypeptide, comprising: (a) cultivating the mutant cell under conditions conducive for production of the polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

The methods used for cultivation and purification of the product of interest may be performed by methods known in the art.

The methods of the present invention for producing an essentially cellobiohydrolase-free product is of particular interest in the production of eukaryotic polypeptides, in particular fungal proteins such as enzymes. The cellobiohydrolase-deficient cells may also be used to express heterologous proteins of pharmaceutical interest such as hormones, growth factors, receptors, and the like. The term “eukaryotic polypeptides” includes not only native polypeptides, but also those polypeptides, e.g., enzymes, which have been modified by amino acid substitutions, deletions or additions, or other such modifications to enhance activity, thermostability, pH tolerance and the like.

In a further aspect, the present invention relates to a protein product essentially free from cellobiohydrolase activity that is produced by a method of the present invention.

### Compositions

The present invention also relates to compositions comprising a polypeptide of the present invention.

The composition may comprise a polypeptide of the present invention as the major enzymatic component, e.g., a mono-component composition. Alternatively, the composition may comprise multiple enzymatic activities, such as one or more (several) enzymes selected from the group consisting of a cellulase, a GH61 polypeptide having cellulolytic enhancing activity, a hemicellulase, an expansin, an esterase, a laccase, a ligninolytic enzyme, a pectinase, a peroxidase, a protease, and a swollenin.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

### Uses

The present invention is also directed to the following methods for using the polypeptides, or compositions thereof.

The present invention also relates to methods for degrading or converting a cellulosic material, comprising: treating the cellulosic material with an enzyme composition in the presence of a polypeptide of the present invention. In one aspect, the method above further comprises recovering the degraded or converted cellulosic material. Soluble products of degradation or conversion of the cellulosic material can be separated from the insoluble cellulosic material using technology well known in the art such as, for example, centrifugation, filtration, and gravity settling.

The present invention also relates to methods for producing a fermentation product, comprising: (a) saccharifying a cellulosic material with an enzyme composition in the presence of a polypeptide of the present invention; (b) fermenting the saccharified cellulosic material with one or more (several) fermenting microorganisms to produce the fermentation product; and (c) recovering the fermentation product from the fermentation.

The present invention also relates to methods of fermenting a cellulosic material, comprising: fermenting the cellulosic material with one or more (several) fermenting microorganisms, wherein the cellulosic material is saccharified with an enzyme composition in the presence of a polypeptide of the present invention. In one aspect, the fermenting of the cellulosic material produces a fermentation product. In another aspect, the method further comprises recovering the fermentation product from the fermentation.

The processing of the cellulosic material according to the present invention can be accomplished using processes conventional in the art. Moreover, the methods of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and cofermentation (SSCF); hybrid hydrolysis and fermentation (HHF); separate hydrolysis and co-fermentation (SHCF); hybrid hydrolysis and co-fermentation (HHCF); and direct microbial conversion (DMC). SHF uses separate process steps to first enzymatically hydrolyze cellulosic material to fermentable sugars, e.g., glucose, cellobiose, cellotriose, and pentose sugars, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the cofermentation of multiple sugars (Sheehan, J., and Himmel, M., 1999, Enzymes, energy and the environment: A strategic perspective on the U.S. Department of Energy's research and development activities for bioethanol, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (several) steps where the same organism is used to produce the enzymes for conversion of the cellulosic material to fermentable sugars and to convert the fermentable sugars into a final product (Lynd, L. R., Weimer, P. J., van Zyl, W. H., and Pretorius, I. S., 2002, Microbial cellulose utilization: Fundamentals and biotechnology, Microbiol. Mol. Biol. Reviews 66: 506-577). It is understood herein that any method known in the art comprising pretreatment, enzymatic hydrolysis (saccharification), fermentation, or a combination thereof, can be used in the practicing the methods of the present invention.

A conventional apparatus can include a fed-batch stirred reactor, a batch stirred reactor, a continuous flow stirred reactor with ultrafiltration, and/or a continuous plug-flow column reactor (Fernanda de Castilhos Corazza, Flávio Faria de Moraes, Gisella Maria Zanin and Ivo Neitzel, 2003, Optimal control in fed-batch reactor for the cellobiose hydrolysis, Acta Scientiarum. Technology 25: 33-38; Gusakov, A. V., and Sinitsyn, A. P., 1985, Kinetics of the enzymatic hydrolysis of cellulose: 1. A mathematical model for a batch reactor process, Enz. Microb. Technol. 7: 346-352), an attrition reactor (Ryu, S. K., and Lee, J. M., 1983, Bioconversion of waste cellulose by using an attrition bioreactor, Biotechnol. Bioeng. 25: 53-65), or a reactor with intensive stirring induced by an electromagnetic field (Gusakov, A. V., Sinitsyn, A. P., Davydkin, I. Y., Davydkin, V. Y., Protas, O. V., 1996, Enhancement of enzymatic cellulose hydrolysis using a novel type of bioreactor with intensive stirring induced by electromagnetic field, Appl. Biochem. Biotechnol. 56: 141-153). Additional reactor types include: fluidized bed, upflow blanket, immobilized, and extruder type reactors for hydrolysis and/or fermentation.

Pretreatment. In practicing the methods of the present invention, any pretreatment process known in the art can be used to disrupt plant cell wall components of the cellulosic material (Chandra et al., 2007, Substrate pretreatment: The key to effective enzymatic hydrolysis of lignocellulosics? Adv. Biochem. Engin./Biotechnol. 108: 67-93; Galbe and Zacchi, 2007, Pretreatment of lignocellulosic materials for efficient bioethanol production, Adv. Biochem. Engin./Biotechnol. 108: 41-65; Hendriks and Zeeman, 2009, Pretreatments to enhance the digestibility of lignocellulosic biomass, Bioresource Technol. 100: 10-18; Mosier et al., 2005, Features of promising technologies for pretreatment of lignocellulosic biomass, Bioresource Technol. 96: 673-686; Taherzadeh and Karimi, 2008, Pretreatment of lignocellulosic wastes to improve ethanol and biogas production: A review, Int. J. of Mol. Sci. 9: 1621-1651; Yang and Wyman, 2008, Pretreatment: the key to unlocking low-cost cellulosic ethanol, Biofuels Bioproducts and Biorefining-Biofpr. 2: 26-40).

The cellulosic material can also be subjected to particle size reduction, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of the cellulosic material to fermentable sugars (even in absence of enzymes).

Steam Pretreatment: In steam pretreatment, cellulosic material is heated to disrupt the plant cell wall components, including lignin, hemicellulose, and cellulose to make the cellulose and other fractions, e.g., hemicellulose, accessible to enzymes. Cellulosic material is passed to or through a reaction vessel where steam is injected to increase the temperature to the required temperature and pressure and is retained therein for the desired reaction time. Steam pretreatment is preferably done at 140-230° C., more preferably 160-200° C., and most preferably 170-190° C., where the optimal temperature range depends on any addition of a chemical catalyst. Residence time for the steam pretreatment is preferably 1-15 minutes, more preferably 3-12 minutes, and most preferably 4-10 minutes, where the optimal residence time depends on temperature range and any addition of a chemical catalyst. Steam pretreatment allows for relatively high solids loadings, so that cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 20020164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

A catalyst such as H₂SO₄ or SO₂ (typically 0.3 to 3% w/w) is often added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762).

Chemical Pretreatment: The term “chemical treatment” refers to any chemical pretreatment that promotes the separation and/or release of cellulose, hemicellulose, and/or lignin. Examples of suitable chemical pretreatment processes include, for example, dilute acid pretreatment, lime pretreatment, wet oxidation, ammonia fiber/freeze explosion (AFEX), ammonia percolation (APR), and organosolv pretreatments.

In dilute acid pretreatment, cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technol. 91: 179-188; Lee et al., 1999, Adv. Biochem. Eng. Biotechnol. 65: 93-115).

Several methods of pretreatment under alkaline conditions can also be used. These alkaline pretreatments include, but are not limited to, lime pretreatment, wet oxidation, ammonia percolation (APR), and ammonia fiber/freeze explosion (AFEX).

Lime pretreatment is performed with calcium carbonate, sodium hydroxide, or ammonia at low temperatures of 85-150° C. and residence times from 1 hour to several days (Wyman et al., 2005, Bioresource Technol. 96: 1959-1966; Mosier et al., 2005, Bioresource Technol. 96: 673-686). WO 2006/110891, WO 2006/11899, WO 2006/11900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technol. 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed at preferably 1-40% dry matter, more preferably 2-30% dry matter, and most preferably 5-20% dry matter, and often the initial pH is increased by the addition of alkali such as sodium carbonate.

A modification of the wet oxidation pretreatment method, known as wet explosion (combination of wet oxidation and steam explosion), can handle dry matter up to 30%. In wet explosion, the oxidizing agent is introduced during pretreatment after a certain residence time. The pretreatment is then ended by flashing to atmospheric pressure (WO 2006/032282).

Ammonia fiber explosion (AFEX) involves treating cellulosic material with liquid or gaseous ammonia at moderate temperatures such as 90-100° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technol. 96: 2014-2018). AFEX pretreatment results in the depolymerization of cellulose and partial hydrolysis of hemicellulose. Lignin-carbohydrate complexes are cleaved.

Organosolv pretreatment delignifies cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose is removed.

Other examples of suitable pretreatment methods are described by Schell et al., 2003, Appl. Biochem. and Biotechnol. Vol. 105-108, p. 69-85, and Mosier et al., 2005, Bioresource Technology 96: 673-686, and U.S. Published Application 2002/0164730.

In one aspect, the chemical pretreatment is preferably carried out as an acid treatment, and more preferably as a continuous dilute and/or mild acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, more preferably 1-4, and most preferably 1-3. In one aspect, the acid concentration is in the range from preferably 0.01 to 20 wt % acid, more preferably 0.05 to 10 wt % acid, even more preferably 0.1 to 5 wt % acid, and most preferably 0.2 to 2.0 wt % acid. The acid is contacted with cellulosic material and held at a temperature in the range of preferably 160-220° C., and more preferably 165-195° C., for periods ranging from seconds to minutes to, e.g., 1 second to 60 minutes.

In another aspect, pretreatment is carried out as an ammonia fiber explosion step (AFEX pretreatment step).

In another aspect, pretreatment takes place in an aqueous slurry. In preferred aspects, cellulosic material is present during pretreatment in amounts preferably between 10-80 wt %, more preferably between 20-70 wt %, and most preferably between 30-60 wt %, such as around 50 wt %. The pretreated cellulosic material can be unwashed or washed using any method known in the art, e.g., washed with water.

Mechanical Pretreatment: The term “mechanical pretreatment” refers to various types of grinding or milling (e.g., dry milling, wet milling, or vibratory ball milling).

Physical Pretreatment: The term “physical pretreatment” refers to any pretreatment that promotes the separation and/or release of cellulose, hemicellulose, and/or lignin from the cellulosic material. For example, physical pretreatment can involve irradiation (e.g., microwave irradiation), steaming/steam explosion, hydrothermolysis, and combinations thereof.

Physical pretreatment can involve high pressure and/or high temperature (steam explosion). In one aspect, high pressure means pressure in the range of preferably about 300 to about 600 psi, more preferably about 350 to about 550 psi, and most preferably about 400 to about 500 psi, such as around 450 psi. In another aspect, high temperature means temperatures in the range of about 100 to about 300° C., preferably about 140 to about 235° C. In a preferred aspect, mechanical pretreatment is performed in a batch-process, steam gun hydrolyzer system that uses high pressure and high temperature as defined above, e.g., a Sunds Hydrolyzer available from Sunds Defibrator AB, Sweden.

Combined Physical and Chemical Pretreatment: Cellulosic material can be pretreated both physically and chemically. For instance, the pretreatment step can involve dilute or mild acid treatment and high temperature and/or pressure treatment. The physical and chemical pretreatments can be carried out sequentially or simultaneously, as desired. A mechanical pretreatment can also be included.

Accordingly, in a preferred aspect, the cellulosic material is subjected to mechanical, chemical, or physical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Physicochemical and biological treatments for enzymatic/microbial conversion of cellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng./Biotechnol. 42: 63-95).

Saccharification. In the hydrolysis step, also known as saccharification, the cellulosic material, e.g., pretreated, is hydrolyzed to break down cellulose and alternatively also hemicellulose to fermentable sugars, such as glucose, cellobiose, xylose, xylulose, arabinose, mannose, galactose, and/or soluble oligosaccharides. The hydrolysis is performed enzymatically by an enzyme composition in the presence of a polypeptide having cellobiohydrolase activity. The enzyme and protein components of the compositions can be added sequentially.

Enzymatic hydrolysis is preferably carried out in a suitable aqueous environment under conditions that can be readily determined by one skilled in the art. In a preferred aspect, hydrolysis is performed under conditions suitable for the activity of the enzyme(s), i.e., optimal for the enzyme(s). The hydrolysis can be carried out as a fed batch or continuous process where the pretreated cellulosic material (substrate) is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 96 hours, more preferably about 16 to about 72 hours, and most preferably about 24 to about 48 hours. The temperature is in the range of preferably about 25° C. to about 70° C., more preferably about 30° C. to about 65° C., and more preferably about 40° C. to 60° C., in particular about 50° C. The pH is in the range of preferably about 3 to about 8, more preferably about 3.5 to about 7, and most preferably about 4 to about 6, in particular about pH 5. The dry solids content is in the range of preferably about 5 to about 50 wt %, more preferably about 10 to about 40 wt %, and most preferably about 20 to about 30 wt %.

The optimum amounts of the enzymes and polypeptides having cellobiohydrolase activity depend on several factors including, but not limited to, the mixture of component cellulolytic enzymes, the cellulosic substrate, the concentration of cellulosic substrate, the pretreatment(s) of the cellulosic substrate, temperature, time, pH, and inclusion of fermenting organism (e.g., yeast for Simultaneous Saccharification and Fermentation).

In one aspect, an effective amount of cellulolytic or hemicellulolytic enzyme protein to cellulosic material is about 0.5 to about 50 mg, preferably at about 0.5 to about 40 mg, more preferably at about 0.5 to about 25 mg, more preferably at about 0.75 to about 20 mg, more preferably at about 0.75 to about 15 mg, even more preferably at about 0.5 to about 10 mg, and most preferably at about 2.5 to about 10 mg per g of cellulosic material.

In another aspect, an effective amount of a polypeptide having cellobiohydrolase activity to cellulosic material is about 0.01 to about 50.0 mg, preferably about 0.01 to about 40 mg, more preferably about 0.01 to about 30 mg, more preferably about 0.01 to about 20 mg, more preferably about 0.01 to about 10 mg, more preferably about 0.01 to about 5 mg, more preferably at about 0.025 to about 1.5 mg, more preferably at about 0.05 to about 1.25 mg, more preferably at about 0.075 to about 1.25 mg, more preferably at about 0.1 to about 1.25 mg, even more preferably at about 0.15 to about 1.25 mg, and most preferably at about 0.25 to about 1.0 mg per g of cellulosic material.

In another aspect, an effective amount of a polypeptide having cellobiohydrolase activity to cellulolytic enzyme protein is about 0.005 to about 1.0 g, preferably at about 0.01 to about 1.0 g, more preferably at about 0.15 to about 0.75 g, more preferably at about 0.15 to about 0.5 g, more preferably at about 0.1 to about 0.5 g, even more preferably at about 0.1 to about 0.5 g, and most preferably at about 0.05 to about 0.2 g per g of cellulolytic enzyme protein.

The enzyme compositions can comprise any protein that is useful in degrading or converting a cellulosic material.

In one aspect, the enzyme composition comprises or further comprises one or more (several) proteins selected from the group consisting of a cellulase, a GH61 polypeptide having cellulolytic enhancing activity, a hemicellulase, an expansin, an esterase, a laccase, a ligninolytic enzyme, a pectinase, a peroxidase, a protease, and a swollenin. In another aspect, the cellulase is preferably one or more (several) enzymes selected from the group consisting of an endoglucanase, a cellobiohydrolase, and a beta-glucosidase. In another aspect, the hemicellulase is preferably one or more (several) enzymes selected from the group consisting of an acetylmannan esterase, an acetyxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

In another aspect, the enzyme composition comprises one or more (several) cellulolytic enzymes. In another aspect, the enzyme composition comprises or further comprises one or more (several) hemicellulolytic enzymes. In another aspect, the enzyme composition comprises one or more (several) cellulolytic enzymes and one or more (several) hemicellulolytic enzymes. In another aspect, the enzyme composition comprises one or more (several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises a polypeptide having cellulolytic enhancing activity. In another aspect, the enzyme composition comprises an endoglucanase and a polypeptide having cellulolytic enhancing activity. In another aspect, the enzyme composition comprises a cellobiohydrolase and a polypeptide having cellulolytic enhancing activity. In another aspect, the enzyme composition comprises a beta-glucosidase and a polypeptide having cellulolytic enhancing activity. In another aspect, the enzyme composition comprises an endoglucanase and a cellobiohydrolase. In another aspect, the enzyme composition comprises an endoglucanase and a beta-glucosidase. In another aspect, the enzyme composition comprises a cellobiohydrolase and a beta-glucosidase. In another aspect, the enzyme composition comprises an endoglucanase, a cellobiohydrolase, and a polypeptide having cellulolytic enhancing activity. In another aspect, the enzyme composition comprises an endoglucanase, a beta-glucosidase, and a polypeptide having cellulolytic enhancing activity. In another aspect, the enzyme composition comprises a cellobiohydrolase, a beta-glucosidase, and a polypeptide having cellulolytic enhancing activity. In another aspect, the enzyme composition comprises an endoglucanase, a cellobiohydrolase, and a beta-glucosidase, and a polypeptide having cellulolytic enhancing activity.

In another aspect, the enzyme composition comprises an acetylmannan esterase. In another aspect, the enzyme composition comprises an acetyxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In a preferred aspect, the xylanase is a Family 10 xylanase. In another aspect, the enzyme composition comprises a xylosidase. In another aspect, the enzyme composition comprises an expansin. In another aspect, the enzyme composition comprises an esterase. In another aspect, the enzyme composition comprises a laccase. In another aspect, the enzyme composition comprises a ligninolytic enzyme. In a preferred aspect, the ligninolytic enzyme is a manganese peroxidase. In another preferred aspect, the ligninolytic enzyme is a lignin peroxidase. In another preferred aspect, the ligninolytic enzyme is a H₂O₂-producing enzyme. In another aspect, the enzyme composition comprises a pectinase. In another aspect, the enzyme composition comprises a peroxidase. In another aspect, the enzyme composition comprises a protease. In another aspect, the enzyme composition comprises a swollenin.

In the methods of the present invention, the enzyme(s) can be added prior to or during fermentation, e.g., during saccharification or during or after propagation of the fermenting microorganism(s).

One or more (several) components of the enzyme composition may be wild-type proteins, recombinant proteins, or a combination of wild-type proteins and recombinant proteins. For example, one or more (several) components may be native proteins of a cell, which is used as a host cell to express recombinantly one or more (several) other components of the enzyme composition. One or more (several) components of the enzyme composition may be produced as monocomponents, which are then combined to form the enzyme composition. The enzyme composition may be a combination of multicomponent and monocomponent protein preparations.

The enzymes used in the methods of the present invention may be in any form suitable for use, such as, for example, a crude fermentation broth with or without cells removed, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The enzymes can be derived or obtained from any suitable origin, including, bacterial, fungal, yeast, plant, or mammalian origin. The term “obtained” means herein that the enzyme may have been isolated from an organism that naturally produces the enzyme as a native enzyme. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

The polypeptide having enzyme activity may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having enzyme activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity.

In a preferred aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having enzyme activity.

In another preferred aspect, the polypeptide is a Streptococcus equisimilis, Streptococcus pyogenes, Streptococcus uberis, or Streptococcus equi subsp. Zooepidemicus polypeptide having enzyme activity.

In another preferred aspect, the polypeptide is a Streptomyces achromogenes, Streptomyces avermitilis, Streptomyces coelicolor, Streptomyces griseus, or Streptomyces lividans polypeptide having enzyme activity.

The polypeptide having enzyme activity may also be a fungal polypeptide, and more preferably a yeast polypeptide such as a Candida, Kluyveromyces, Pichia, Saccharomyces, Schizosaccharomyces, or Yarrowia polypeptide having enzyme activity; or more preferably a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme activity.

In another preferred aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having enzyme activity.

Chemically modified or protein engineered mutants of the polypeptides having enzyme activity may also be used.

One or more (several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic enzymes may also be prepared by purifying such a protein from a fermentation broth.

In one aspect, the one or more (several) cellulolytic enzymes comprise a commercial cellulolytic enzyme preparation. Examples of commercial cellulolytic enzyme preparations suitable for use in the present invention include, for example, CELLIC™ CTec (Novozymes A/S), CELLIC™ CTec2 (Novozymes A/S), CELLUCLAST™ (Novozymes A/S), NOVOZYM™ 188 (Novozymes A/S), CELLUZYME™ (Novozymes A/S), CEREFLO™ (Novozymes A/S), and ULTRAFLO™ (Novozymes A/S), ACCELERASE™ (Genencor Int.), LAMINEX™ (Genencor Int.), SPEZYME™ CP (Genencor Int.), ROHAMENT™ 7069 W (Röhm GmbH), FIBREZYME® LDI (Dyadic International, Inc.), FIBREZYME® LBR (Dyadic International, Inc.), or VISCOSTAR® 150L (Dyadic International, Inc.). The cellulase enzymes are added in amounts effective from about 0.001 to about 5.0 wt % of solids, more preferably from about 0.025 to about 4.0 wt % of solids, and most preferably from about 0.005 to about 2.0 wt % of solids.

Examples of bacterial endoglucanases that can be used in the methods of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the present invention include, but are not limited to, a Trichoderma reesei endoglucanase I (Penttila et al., 1986, Gene 45: 253-263; Trichoderma reesei Cel7B endoglucanase I; GENBANK™ accession no. M15665; SEQ ID NO: 4); Trichoderma reesei endoglucanase II (Saloheimo, et al., 1988, Gene 63:11-22; Trichoderma reesei Cel5A endoglucanase II; GENBANK™ accession no. M19373; SEQ ID NO: 6); Trichoderma reesei endoglucanase III (Okada et al., 1988, Appl. Environ. Microbiol. 64: 555-563; GENBANK™ accession no. AB003694; SEQ ID NO: 8); Trichoderma reesei endoglucanase V (Saloheimo et al., 1994, Molecular Microbiology 13: 219-228; GENBANK™ accession no. Z33381; SEQ ID NO: 10); Aspergillus aculeatus endoglucanase (Ooi et al., 1990, Nucleic Acids Research 18: 5884); Aspergillus kawachii endoglucanase (Sakamoto et al., 1995, Current Genetics 27: 435-439); Erwinia carotovara endoglucanase (Saarilahti et al., 1990, Gene 90: 9-14); Fusarium oxysporum endoglucanase (GENBANK™ accession no. L29381); Humicola grisea var. thermoidea endoglucanase (GENBANK™ accession no. AB003107); Melanocarpus albomyces endoglucanase (GENBANK™ accession no. MAL515703); Neurospora crassa endoglucanase (GENBANK™ accession no. XM_{—}324477); Humicola insolens endoglucanase V (SEQ ID NO: 12); Myceliophthora thermophila CBS117.65 endoglucanase (SEQ ID NO: 14); basidiomycete CBS 495.95 endoglucanase (SEQ ID NO: 16); basidiomycete CBS 494.95 endoglucanase (SEQ ID NO: 18); Thielavia terrestris NRRL 8126 CEL6B endoglucanase (SEQ ID NO: 20); Thielavia terrestris NRRL 8126 CEL6C endoglucanase (SEQ ID NO: 22); Thielavia terrestris NRRL 8126 CEL7C endoglucanase (SEQ ID NO: 24); Thielavia terrestris NRRL 8126 CEL7E endoglucanase (SEQ ID NO: 26); Thielavia terrestris NRRL 8126 CEL7F endoglucanase (SEQ ID NO: 28); Cladorrhinum foecundissimum ATCC 62373 CEL7A endoglucanase (SEQ ID NO: 30); and Trichoderma reesei strain No. VTT-D-80133 endoglucanase (SEQ ID NO: 32; GENBANK™ accession no. M15665). The endoglucanases of SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32, described above are encoded by the mature polypeptide coding sequence of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31, respectively.

Examples of cellobiohydrolases useful in the present invention include, but are not limited to, Trichoderma reesei cellobiohydrolase I (SEQ ID NO: 34); Trichoderma reesei cellobiohydrolase II (SEQ ID NO: 36); Humicola insolens cellobiohydrolase I (SEQ ID NO: 38); Myceliophthora thermophila cellobiohydrolase II (SEQ ID NO: 40 and SEQ ID NO: 42); Thielavia terrestris cellobiohydrolase II (CEL6A) (SEQ ID NO: 44); Chaetomium thermophilum cellobiohydrolase I (SEQ ID NO: 46); and Chaetomium thermophilum cellobiohydrolase II (SEQ ID NO: 48), Aspergillus fumigatus cellobiohydrolase I (SEQ ID NO: 50), and Aspergillus fumigatus cellobiohydrolase II (SEQ ID NO: 52). The cellobiohydrolases of SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, and SEQ ID NO: 52, described above are encoded by the mature polypeptide coding sequence of SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, and SEQ ID NO: 51, respectively.

Examples of beta-glucosidases useful in the present invention include, but are not limited to, Aspergillus oryzae beta-glucosidase (SEQ ID NO: 54); Aspergillus fumigatus beta-glucosidase (SEQ ID NO: 56); Penicillium brasilianum IBT 20888 beta-glucosidase (SEQ ID NO: 58); Aspergillus niger beta-glucosidase (SEQ ID NO: 60); and Aspergillus aculeatus beta-glucosidase (SEQ ID NO: 62). The beta-glucosidases of SEQ ID NO: 54, SEQ ID NO: 56, SEQ ID NO: 58, SEQ ID NO: 60, and SEQ ID NO: 62, described above are encoded by the mature polypeptide coding sequence of SEQ ID NO: 53, SEQ ID NO: 55, SEQ ID NO: 57, SEQ ID NO: 59, and SEQ ID NO: 61, respectively.

Examples of other beta-glucosidases useful in the present invention include a Aspergillus oryzae beta-glucosidase variant fusion protein of SEQ ID NO: 64 or the Aspergillus oryzae beta-glucosidase fusion protein of SEQ ID NO: 66. The beta-glucosidase fusion proteins of SEQ ID NO: 64 and SEQ ID NO: 66 are encoded by SEQ ID NO: 63 and SEQ ID NO: 65, respectively.

The Aspergillus oryzae beta-glucosidase can be obtained according to WO 2002/095014. The Aspergillus fumigatus beta-glucosidase can be obtained according to WO 2005/047499. The Penicillium brasilianum beta-glucosidase can be obtained according to WO 2007/019442. The Aspergillus niger beta-glucosidase can be obtained according to Dan et al., 2000, J. Biol. Chem. 275: 4973-4980. The Aspergillus aculeatus beta-glucosidase can be obtained according to Kawaguchi et al., 1996, Gene 173: 287-288.

Other useful endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Other cellulolytic enzymes that may be useful in the present invention are described in EP 495,257, EP 531,315, EP 531,372, WO 89/09259, WO 94/07998, WO 95/24471, WO 96/11262, WO 96/29397, WO 96/034108, WO 97/14804, WO 98/08940, WO 98/012307, WO 98/13465, WO 98/015619, WO 98/015633, WO 98/028411, WO 99/06574, WO 99/10481, WO 99/025846, WO 99/025847, WO 99/031255, WO 2000/009707, WO 2002/050245, WO 2002/0076792, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. No. 4,435,307, U.S. Pat. No. 5,457,046, U.S. Pat. No. 5,648,263, U.S. Pat. No. 5,686,593, U.S. Pat. No. 5,691,178, U.S. Pat. No. 5,763,254, and U.S. Pat. No. 5,776,757.

In the methods of the present invention, any GH61 polypeptide having cellulolytic enhancing activity can be used.

In a first aspect, the polypeptide having cellulolytic enhancing activity comprises the following motifs:

[ILMV]-P-X(4,5)-G-X-Y-[ILMV]-X-R-X-[EQ]-X(4)-[HNQ] and [FW]-[TF]-K-[AIV],

wherein X is any amino acid, X(4,5) is any amino acid at 4 or 5 contiguous positions, and X(4) is any amino acid at 4 contiguous positions.

The polypeptide comprising the above-noted motifs may further comprise:

H-X(1,2)-G-P-X(3)-[YW]-[AILMV],

[EQ]-X-Y-X(2)-C-X-[EHQN]-[FILV]-X-[ILV], or

H-X(1,2)-G-P-X(3)-[YW]-[AILMV] and [EQ]-X-Y-X(2)-C-X-[EHQN]-[FILV]-X-[ILV],

wherein X is any amino acid, X(1,2) is any amino acid at 1 position or 2 contiguous positions, X(3) is any amino acid at 3 contiguous positions, and X(2) is any amino acid at 2 contiguous positions. In the above motifs, the accepted IUPAC single letter amino acid abbreviation is employed.

In a preferred aspect, the polypeptide having cellulolytic enhancing activity further comprises H-X(1,2)-G-P-X(3)-[YW]-[AILMV]. In another preferred aspect, the isolated polypeptide having cellulolytic enhancing activity further comprises [EQ]-X-Y-X(2)-C-X-[EHQN]-[FILV]-X-[ILV]. In another preferred aspect, the polypeptide having cellulolytic enhancing activity further comprises H-X(1,2)-G-P-X(3)-[YW]-[AILMV] and [EQ]-X-Y-X(2)-C-X-[EHQN]-[FILV]-X-[ILV].

In a second aspect, the polypeptide having cellulolytic enhancing activity comprises the following motif:

[ILMV]-P-x(4,5)-G-x-Y-[ILMV]-x-R-x-[EQ]-x(3)-A-[HNQ],

wherein x is any amino acid, x(4,5) is any amino acid at 4 or 5 contiguous positions, and x(3) is any amino acid at 3 contiguous positions. In the above motif, the accepted IUPAC single letter amino acid abbreviation is employed.

In a third aspect, the polypeptide having cellulolytic enhancing activity comprises an amino acid sequence that has a degree of identity to the mature polypeptide of SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, or SEQ ID NO: 130 of at least 60%, e.g., at least 65%, at least 70%, at least 75%, at least 80%, at least 85%, at least 90%, at least 91%, at least 92%, at least 93%, at least 94%, or at least 95%, at least 96%, at least 97%, at least 98%, at least 99%, or at least 100%.

In a fourth aspect, the polypeptide having cellulolytic enhancing activity is encoded by a polynucleotide that hybridizes under at least very low stringency conditions, preferably at least low stringency conditions, more preferably at least medium stringency conditions, more preferably at least medium-high stringency conditions, even more preferably at least high stringency conditions, and most preferably at least very high stringency conditions with (i) the mature polypeptide coding sequence of SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, or SEQ ID NO: 129, (ii) the cDNA sequence contained in the mature polypeptide coding sequence of SEQ ID NO: 73, SEQ ID NO: 75, SEQ ID NO: 77, or SEQ ID NO: 81, or the genomic DNA sequence comprising the mature polypeptide coding sequence of SEQ ID NO: 67, SEQ ID NO: 69, SEQ ID NO: 71, SEQ ID NO: 79, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, or SEQ ID NO: 129, (iii) a subsequence of (i) or (ii), or (iv) a full-length complementary strand of (i), (ii), or (iii) (J. Sambrook, E. F. Fritsch, and T. Maniatus, 1989, supra). A subsequence of the mature polypeptide coding sequence of SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, or SEQ ID NO: 129 contains at least 100 contiguous nucleotides or preferably at least 200 contiguous nucleotides. Moreover, the subsequence may encode a polypeptide fragment that has cellulolytic enhancing activity.

In a fifth aspect, the polypeptide having cellulolytic enhancing activity is encoded by a polynucleotide comprising or consisting of a nucleotide sequence that has a degree of identity to the mature polypeptide coding sequence of SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, or SEQ ID NO: 129 of preferably at least 60%, more preferably at least 65%, more preferably at least 70%, more preferably at least 75%, more preferably at least 80%, more preferably at least 85%, even more preferably at least 90%, most preferably at least 91%, at least 92%, at least 93%, at least 94%, or at least 95%, and even most preferably at least 96%, at least 97%, at least 98%, at least 99%, or at least 100%.

In a sixth aspect, the polypeptide having cellulolytic enhancing activity is an artificial variant comprising a substitution, deletion, and/or insertion of one or more (or several) amino acids of the mature polypeptide of SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, or SEQ ID NO: 130; or a homologous sequence thereof.

Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cellulolytic enhancing activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to the parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The total number of amino acid substitutions, deletions and/or insertions of the mature polypeptide of SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, or SEQ ID NO: 130 is not more than 4, e.g., 1, 2, 3, or 4.

In one aspect, the one or more (several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC™ HTec (Novozymes A/S), CELLIC™ HTec2 (Novozymes A/S), VISCOZYME® (Novozymes A/S), ULTRAFLO® (Novozymes A/S), PULPZYME® HC (Novozymes A/S), MULTIFECT® Xylanase (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK).

Examples of xylanases useful in the methods of the present invention include, but are not limited to, Aspergillus aculeatus xylanase (GeneSeqP:AAR63790; WO 94/21785), Aspergillus fumigatus xylanases (WO 2006/078256; xyl 3 SEQ ID NO: 131 [DNA sequence] and SEQ ID NO: 132 [deduced amino acid sequence]), and Thielavia terrestris NRRL 8126 xylanases (WO 2009/079210).

Examples of beta-xylosidases useful in the methods of the present invention include, but are not limited to, Trichoderma reesei beta-xylosidase (UniProtKB/TrEMBL accession number Q92458; SEQ ID NO: 133 [DNA sequence] and SEQ ID NO: 134 [deduced amino acid sequence]), Talaromyces emersonii (SwissProt accession number Q8×212), and Neurospora crassa (SwissProt accession number Q7SOW4).

Examples of acetylxylan esterases useful in the methods of the present invention include, but are not limited to, Hypocrea jecorina acetylxylan esterase (WO 2005/001036), Neurospora crassa acetylxylan esterase (UniProt accession number q7s259), Thielavia terrestris NRRL 8126 acetylxylan esterase (WO 2009/042846), Chaetomium globosum acetylxylan esterase (Uniprot accession number Q2GWX4), Chaetomium gracile acetylxylan esterase (GeneSeqP accession number AAB82124), Phaeosphaeria nodorum acetylxylan esterase (Uniprot accession number QOUHJ1), and Humicola insolens DSM 1800 acetylxylan esterase (WO 2009/073709).

Examples of ferulic acid esterases useful in the methods of the present invention include, but are not limited to, Humicola insolens DSM 1800 feruloyl esterase (WO 2009/076122), Neurospora crassa feruloyl esterase (UniProt accession number Q9HGR3), and Neosartorya fischeri feruloyl esterase (UniProt Accession number A1D9T4).

Examples of arabinofuranosidases useful in the methods of the present invention include, but are not limited to, Humicola insolens DSM 1800 arabinofuranosidase (WO 2009/073383) and Aspergillus niger arabinofuranosidase (GeneSeqP accession number AAR94170).

Examples of alpha-glucuronidases useful in the methods of the present invention include, but are not limited to, Aspergillus clavatus alpha-glucuronidase (UniProt accession number alcc12), Trichoderma reesei alpha-glucuronidase (Uniprot accession number Q99024), Talaromyces emersonii alpha-glucuronidase (UniProt accession number Q8×211), Aspergillus niger alpha-glucuronidase (Uniprot accession number Q96WX9), Aspergillus terreus alpha-glucuronidase (SwissProt accession number Q0CJP9), and Aspergillus fumigatus alpha-glucuronidase (SwissProt accession number Q4WW45).

The enzymes and proteins used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, NY, 1986).

The fermentation can be any method of cultivation of a cell resulting in the expression or isolation of an enzyme. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Fermentation. The fermentable sugars obtained from the hydrolyzed cellulosic material can be fermented by one or more (several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous, as described herein.

Any suitable hydrolyzed cellulosic material can be used in the fermentation step in practicing the present invention. The material is generally selected based on the desired fermentation product, i.e., the substance to be obtained from the fermentation, and the process employed, as is well known in the art.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be C₆ and/or C₅ fermenting organisms, or a combination thereof. Both C₆ and C₅ fermenting organisms are well known in the art. Suitable fermenting microorganisms are able to ferment, i.e., convert, sugars, such as glucose, xylose, xylulose, arabinose, maltose, mannose, galactose, or oligosaccharides, directly or indirectly into the desired fermentation product.

Examples of bacterial and fungal fermenting organisms producing ethanol are described by Lin et al., 2006, Appl. Microbiol. Biotechnol. 69: 627-642.

Examples of fermenting microorganisms that can ferment C₆ sugars include bacterial and fungal organisms, such as yeast. Preferred yeast includes strains of the Saccharomyces spp., preferably Saccharomyces cerevisiae.

Examples of fermenting organisms that can ferment Cₛ sugars include bacterial and fungal organisms, such as some yeast. Preferred Cₛ fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis, or Candida utilis.

Other fermenting organisms include strains of Zymomonas, such as Zymomonas mobilis; Hansenula, such as Hansenula anomala; Kluyveromyces, such as K fragilis; Schizosaccharomyces, such as S. pombe; E. coli, especially E. coli strains that have been genetically modified to improve the yield of ethanol; Clostridium, such as Clostridium acetobutylicum, Chlostridium thermocellum, and Chlostridium phytofermentans; Geobacillus sp.; Thermoanaerobacter, such as Thermoanaerobacter saccharolyticum; and Bacillus, such as Bacillus coagulans.

In a preferred aspect, the yeast is a Saccharomyces spp. In a more preferred aspect, the yeast is Saccharomyces cerevisiae. In another more preferred aspect, the yeast is Saccharomyces distaticus. In another more preferred aspect, the yeast is Saccharomyces uvarum. In another preferred aspect, the yeast is a Kluyveromyces. In another more preferred aspect, the yeast is Kluyveromyces marxianus. In another more preferred aspect, the yeast is Kluyveromyces fragilis. In another preferred aspect, the yeast is a Candida. In another more preferred aspect, the yeast is Candida boidinii. In another more preferred aspect, the yeast is Candida brassicae. In another more preferred aspect, the yeast is Candida diddensii. In another more preferred aspect, the yeast is Candida pseudotropicalis. In another more preferred aspect, the yeast is Candida utilis. In another preferred aspect, the yeast is a Clavispora. In another more preferred aspect, the yeast is Clavispora lusitaniae. In another more preferred aspect, the yeast is Clavispora opuntiae. In another preferred aspect, the yeast is a Pachysolen. In another more preferred aspect, the yeast is Pachysolen tannophilus. In another preferred aspect, the yeast is a Pichia. In another more preferred aspect, the yeast is a Pichia stipitis. In another preferred aspect, the yeast is a Bretannomyces. In another more preferred aspect, the yeast is Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212).

Bacteria that can efficiently ferment hexose and pentose to ethanol include, for example, Zymomonas mobilis, Clostridium acetobutylicum, Clostridium thermocellum, Chlostridium phytofermentans, Geobacillus sp., Thermoanaerobacter saccharolyticum, and Bacillus coagulans (Philippidis, 1996, supra).

In a preferred aspect, the bacterium is a Zymomonas. In a more preferred aspect, the bacterium is Zymomonas mobilis. In another preferred aspect, the bacterium is a Clostridium. In another more preferred aspect, the bacterium is Clostridium thermocellum.

Commercially available yeast suitable for ethanol production includes, e.g., ETHANOL RED™ yeast (Fermentis/Lesaffre, USA), FALI™ (Fleischmann's Yeast, USA), SUPERSTART™ and THERMOSACC™ fresh yeast (Ethanol Technology, WI, USA), BIOFERM™ AFT and XR (NABC—North American Bioproducts Corporation, GA, USA), GERT STRAND™ (Gert Strand AB, Sweden), and FERMIOL™ (DSM Specialties).

In a preferred aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (cofermentation) (Chen and Ho, 1993, Cloning and improving the expression of Pichia stipitis xylose reductase gene in Saccharomyces cerevisiae, Appl. Biochem. Biotechnol. 39-40: 135-147; Ho et al., 1998, Genetically engineered Saccharomyces yeast capable of effectively cofermenting glucose and xylose, Appl. Environ. Microbiol. 64: 1852-1859; Kotter and Ciriacy, 1993, Xylose fermentation by Saccharomyces cerevisiae, Appl. Microbiol. Biotechnol. 38: 776-783; Walfridsson et al., 1995, Xylose-metabolizing Saccharomyces cerevisiae strains overexpressing the TKL1 and TAL1 genes encoding the pentose phosphate pathway enzymes transketolase and transaldolase, Appl. Environ. Microbiol. 61: 4184-4190; Kuyper et al., 2004, Minimal metabolic engineering of Saccharomyces cerevisiae for efficient anaerobic xylose fermentation: a proof of principle, FEMS Yeast Research 4: 655-664; Beall et al., 1991, Parametric studies of ethanol production from xylose and other sugars by recombinant Escherichia coli, Biotech. Bioeng. 38: 296-303; Ingram et al., 1998, Metabolic engineering of bacteria for ethanol production, Biotechnol. Bioeng. 58: 204-214; Zhang et al., 1995, Metabolic engineering of a pentose metabolism pathway in ethanologenic Zymomonas mobilis, Science 267: 240-243; Deanda et al., 1996, Development of an arabinose-fermenting Zymomonas mobilis strain by metabolic pathway engineering, Appl. Environ. Microbiol. 62: 4465-4470; WO 2003/062430, xylose isomerase).

In a preferred aspect, the genetically modified fermenting microorganism is Saccharomyces cerevisiae. In another preferred aspect, the genetically modified fermenting microorganism is Zymomonas mobilis. In another preferred aspect, the genetically modified fermenting microorganism is Escherichia coli. In another preferred aspect, the genetically modified fermenting microorganism is Klebsiella oxytoca. In another preferred aspect, the genetically modified fermenting microorganism is Kluyveromyces sp.

It is well known in the art that the organisms described above can also be used to produce other substances, as described herein.

The fermenting microorganism is typically added to the degraded lignocellulose or hydrolysate and the fermentation is performed for about 8 to about 96 hours, such as about 24 to about 60 hours. The temperature is typically between about 26° C. to about 60° C., in particular about 32° C. or 50° C., and at about pH 3 to about pH 8, such as around pH 4-5, 6, or 7.

In a preferred aspect, the yeast and/or another microorganism is applied to the degraded cellulosic material and the fermentation is performed for about 12 to about 96 hours, such as typically 24-60 hours. In a preferred aspect, the temperature is preferably between about 20° C. to about 60° C., more preferably about 25° C. to about 50° C., and most preferably about 32° C. to about 50° C., in particular about 32° C. or 50° C., and the pH is generally from about pH 3 to about pH 7, preferably around pH 4-7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

For ethanol production, following the fermentation the fermented slurry is distilled to extract the ethanol. The ethanol obtained according to the methods of the invention can be used as, e.g., fuel ethanol, drinking ethanol, i.e., potable neutral spirits, or industrial ethanol.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation products: A fermentation product can be any substance derived from the fermentation. The fermentation product can be, without limitation, an alcohol (e.g., arabinitol, butanol, ethanol, glycerol, methanol, 1,3-propanediol, sorbitol, and xylitol); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); a ketone (e.g., acetone); an amino acid (e.g., aspartic acid, glutamic acid, glycine, lysine, serine, and threonine); and a gas (e.g., methane, hydrogen (H₂), carbon dioxide (CO₂), and carbon monoxide (CO)). The fermentation product can also be protein as a high value product.

In a preferred aspect, the fermentation product is an alcohol. It will be understood that the term “alcohol” encompasses a substance that contains one or more hydroxyl moieties. In a more preferred aspect, the alcohol is arabinitol. In another more preferred aspect, the alcohol is butanol. In another more preferred aspect, the alcohol is ethanol. In another more preferred aspect, the alcohol is glycerol. In another more preferred aspect, the alcohol is methanol. In another more preferred aspect, the alcohol is 1,3-propanediol. In another more preferred aspect, the alcohol is sorbitol. In another more preferred aspect, the alcohol is xylitol. See, for example, Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Silveira, M. M., and Jonas, R., 2002, The biotechnological production of sorbitol, Appl. Microbiol. Biotechnol. 59: 400-408; Nigam, P., and Singh, D., 1995, Processes for fermentative production of xylitol—a sugar substitute, Process Biochemistry 30 (2): 117-124; Ezeji, T. C., Qureshi, N. and Blaschek, H. P., 2003, Production of acetone, butanol and ethanol by Clostridium beijerinckii BA101 and in situ recovery by gas stripping, World Journal of Microbiology and Biotechnology 19 (6): 595-603.

In another preferred aspect, the fermentation product is an organic acid. In another more preferred aspect, the organic acid is acetic acid. In another more preferred aspect, the organic acid is acetonic acid. In another more preferred aspect, the organic acid is adipic acid. In another more preferred aspect, the organic acid is ascorbic acid. In another more preferred aspect, the organic acid is citric acid. In another more preferred aspect, the organic acid is 2,5-diketo-D-gluconic acid. In another more preferred aspect, the organic acid is formic acid. In another more preferred aspect, the organic acid is fumaric acid. In another more preferred aspect, the organic acid is glucaric acid. In another more preferred aspect, the organic acid is gluconic acid. In another more preferred aspect, the organic acid is glucuronic acid. In another more preferred aspect, the organic acid is glutaric acid. In another preferred aspect, the organic acid is 3-hydroxypropionic acid. In another more preferred aspect, the organic acid is itaconic acid. In another more preferred aspect, the organic acid is lactic acid. In another more preferred aspect, the organic acid is malic acid. In another more preferred aspect, the organic acid is malonic acid. In another more preferred aspect, the organic acid is oxalic acid. In another more preferred aspect, the organic acid is propionic acid. In another more preferred aspect, the organic acid is succinic acid. In another more preferred aspect, the organic acid is xylonic acid. See, for example, Chen, R., and Lee, Y. Y., 1997, Membrane-mediated extractive fermentation for lactic acid production from cellulosic biomass, Appl. Biochem. Biotechnol. 63-65: 435-448.

In another preferred aspect, the fermentation product is a ketone. It will be understood that the term “ketone” encompasses a substance that contains one or more ketone moieties. In another more preferred aspect, the ketone is acetone. See, for example, Qureshi and Blaschek, 2003, supra.

In another preferred aspect, the fermentation product is an amino acid. In another more preferred aspect, the organic acid is aspartic acid. In another more preferred aspect, the amino acid is glutamic acid. In another more preferred aspect, the amino acid is glycine. In another more preferred aspect, the amino acid is lysine. In another more preferred aspect, the amino acid is serine. In another more preferred aspect, the amino acid is threonine. See, for example, Richard, A., and Margaritis, A., 2004, Empirical modeling of batch fermentation kinetics for poly(glutamic acid) production and other microbial biopolymers, Biotechnology and Bioengineering 87 (4): 501-515.

In another preferred aspect, the fermentation product is a gas. In another more preferred aspect, the gas is methane. In another more preferred aspect, the gas is H₂. In another more preferred aspect, the gas is CO₂. In another more preferred aspect, the gas is CO. See, for example, Kataoka, N., A. Miya, and K. Kiriyama, 1997, Studies on hydrogen production by continuous culture system of hydrogen-producing anaerobic bacteria, Water Science and Technology 36 (6-7): 41-47; and Gunaseelan V. N. in Biomass and Bioenergy, Vol. 13 (1-2), pp. 83-114, 1997, Anaerobic digestion of biomass for methane production: A review.

Recovery. The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### Signal Peptide

The present invention also relates to an isolated polynucleotide encoding a signal peptide comprising or consisting of amino acids 1 to 18 of SEQ ID NO: 2. The polynucleotide may further comprise a gene encoding a protein, which is operably linked to the signal peptide and/or propeptide. The protein is preferably foreign to the signal peptide. In one aspect, the polynucleotide for the signal peptide is nucleotides 1 to 54 of SEQ ID NO: 1.

The present invention also relates to nucleic acid constructs, expression vectors and recombinant host cells comprising such polynucleotides.

The present invention also relates to methods of producing a protein, comprising: (a) cultivating a recombinant host cell comprising such polynucleotide; and (b) recovering the protein.

The protein may be native or heterologous to a host cell. The term “protein” is not meant herein to refer to a specific length of the encoded product and, therefore, encompasses peptides, oligopeptides, and polypeptides. The term “protein” also encompasses two or more polypeptides combined to form the encoded product. The proteins also include hybrid polypeptides and fused polypeptides.

Preferably, the protein is a hormone or variant thereof, enzyme, receptor or portion thereof, antibody or portion thereof, or reporter. For example, the protein may be an oxidoreductase, transferase, hydrolase, lyase, isomerase, or ligase such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, another lipase, mannosidase, mutanase, oxidase, pectinolytic enzyme, peroxidase, phytase, polyphenoloxidase, proteolytic enzyme, ribonuclease, transglutaminase or xylanase.

The gene may be obtained from any prokaryotic, eukaryotic, or other source.

The present invention is further described by the following examples that should not be construed as limiting the scope of the invention.

### EXAMPLES

Chemicals used as buffers and substrates were commercial products of at least reagent grade.

### Strains

Aspergillus aculeatus strain NN000525 (IAM 2445, IAM Culture Collection, Institute of Molecular and Cellular Biosciences, The University of Tokyo) was used as a source of a GH6 polypeptide having cellobiohydrolase activity. Aspergillus oryzae JaL355 strain (WO 2005/070962) was used for expression of the Aspergillus aculeatus GH6 polypeptide having cellobiohydrolase activity.

### Media

Shake flask medium was composed of 15 g of glucose, 4 g of K₂HPO₄, 1 g of NaCl, 0.2 g of MgSO₄.7H₂O, 2 g of MES free acid, 1 g of Bacto Peptone, 5 g of yeast extract, 2.5 g of citric acid, 0.2 g of CaCl₂.2H₂O, 5 g of NH₄NO₃, 1 ml of trace elements solution, and deionized water to 1 liter.

Trace elements solution was composed of 1.2 g of FeSO₄.7H₂O, 10 g of ZnSO₄.7H₂O, 0.7 g of MnSO₄.H₂O, 0.4 g of CuSO₄.5H₂O, 0.4 g of Na₂B₄O₇.10H₂O, 0.8 g of Na₂MoO₂.2H₂O, and deionized water to 1 liter.

PDA plates were composed of 39 grams of potato dextrose agar and deionized water to 1 liter.

NNCYP-PCS medium was composed of 1 g of NaCl, 5 g of NH₄NO₃, 2 g of MES hydrate, 2.75 g of citric acid, 0.2 g of CaCl₂.H₂O, 5 g of bacto peptone, 5 g of yeast extract, 0.2 g of MgSO₄.7H₂O, 4 g of K₂HPO₄, 1 ml COVE trace metals solution, 2 g of dextrose, 5% w/v PCS (dilute acid pretreated corn stover pH 5), and deionized water to 1 liter.

COVE trace metals solution was composed 0.04 g of Na₂B₄O₇10H₂O, 0.4 g of CuSO₄.5H₂O, 1.2 g of FeSO₄.7H₂O, 0.7 g of MnSO₄.H₂O, 0.8 g of Na₂MoO₄.2H₂O, 10 g of ZnSO₄.7H₂O), and deionized water to 1 liter.

LB plates were composed of 10 g of tryptone, 5 g of yeast extract, 10 g of sodium chloride, 15 g of agar, and deionized water to 1 liter.

YP medium was composed of 10 g of yeast extract, 20 g of Bacto peptone, and deionized water to 1 liter.

YPM medium was composed of 10 g of yeast extract, 20 g of Bacto peptone, 20 g of maltose, and deionized water to 1 liter.

### Example 1

### Growth of Wild-Type Aspergillus aculeatus

Aspergillus aculeatus strain NN000525 was inoculated into 100 ml of shake flask medium in a 500 ml shake flask using two plugs from a PDA plate and incubated at 45° C. on an orbital shaker at 200 rpm for 48 hours. Fifty ml of the shake flask medium was used to inoculate a 2 liter fermentation vessel.

Fermentation batch medium was composed of 5 g of yeast extract, 176 g of powdered cellulose, 2 g of glucose, 1 g of NaCl, 1 g of Bacto Peptone, 4 g of K₂HPO₄, 0.2 g of CaCl₂.2H₂O, 0.2 g of MgSO₄.7H₂O, 2.5 g of citric acid, 5 g of NH₄NO₃, 1.8 ml of anti-foam, 1 ml of trace elements solution, and deionized water to 1 liter. Fermentation feed was composed of water and antifoam.

A total of 1.8 liters of the fermentation batch medium was added to a two liter glass jacketed fermentor (Applikon Biotechnology, Schiedam, Netherlands). Fermentation feed medium was dosed at a rate of 4 g/l/hr for a period of 72 hours. The fermentation vessel was maintained at a temperature of 45° C. and pH was controlled using an Applikon 1030 control system (Applikon Biotechnology, Schiedam, Netherlands) to a set-point of 5.6+/−0.1. Air was added to the vessel at a rate of 1 vvm and the broth was agitated by Rushton impeller rotating at 1100 to 1300 rpm. At the end of the fermentation, whole broth was harvested from the vessel and centrifuged at 3000×g to remove the biomass.

### Example 2

### Purification of Native Cel6A Cellobiohydrolase from Wild-Type Aspergillus aculeatus Whole Broth

The harvested A. aculeatus broth obtained in Example 1 was centrifuged in 500 ml bottles at 13,000×g for 20 minutes at 4° C. and then sterile filtered using a 0.22 μm polyethersulfone membrane (Millipore, Bedford, Mass., USA). The filtered broth was concentrated and buffer exchanged with 20 mM Tris-HCl pH 8.5 using a tangential flow concentrator (Pall Filtron, Northborough, Mass., USA) equipped with a 10 kDa polyethersulfone membrane at approximately 20 psi. To decrease the amount of pigment, the concentrate was applied to a 60 ml Q SEPHAROSE™ Big Bead column (GE Healthcare, Piscataway, N.J., USA) equilibrated with 20 mM Tris-HCl pH 8.5, and step eluted with equilibration buffer containing 0 to 600 mM NaCl. Flow-through and eluate fractions were examined on 8-16% CRITERION™ SDS-PAGE gels (Bio-Rad Laboratories, Inc., Hercules, Calif., USA) stained with GELCODE® Blue Stain Reagent (Thermo Fisher Scientific, Waltham, Mass., USA). The eluate fraction contained A. aculeatus Cel6A cellobiohydrolase as judged by the presence of a 70 kDa band corresponding to the apparent molecular weight of the Cel6A cellobiohydrolase.

The eluate fraction was concentrated using an Amicon ultrafiltration device (Millipore, Bedford, Mass., USA; 10 kDa polyethersulfone membrane, 40 psi, 4° C.) and desalted (HIPREP™ 26/10 desalting columns, GE Healthcare, Piscataway, N.J., USA) into 20 mM Tris-HCl pH 8.5. The desalted material was loaded onto a MONO Q™ column (HR 16/10, GE Healthcare, Piscataway, N.J., USA) equilibrated with 20 mM Tris-HCl pH 8.5. Bound proteins were eluted with a salt gradient (20 column volumes) from 0 M NaCl to 600 mM NaCl in 20 mM Tris-HCl pH 8.5. Fractions were examined by 8-16% SDS-PAGE gels as described above and revealed that the Aspergillus aculeatus Cel6A cellobiohydrolase eluted at approximately 50 mM NaCl.

Fractions containing Cel6A cellobiohydrolase were pooled and mixed with an equal volume of 20 mM Tris-HCl pH 7.5 containing 3.4 M ammonium sulfate for a final concentration of 1.7 M ammonium sulfate. The sample was filtered (0.2 μM syringe filter, polyethersulfone membrane, Whatman, Maidstone, United Kingdom) to remove particulate matter prior to loading onto a 20 ml SOURCE™ 15PHE column (GE Healthcare, Piscataway, N.J., USA) equilibrated with 1.7 M ammonium sulfate in 20 mM Tris-HCl pH 7.5. Bound proteins were eluted with a decreasing salt gradient (15 column volumes) from 1.7 M ammonium sulfate to 0 M ammonium sulfate in 20 mM Tris-HCl pH 7.5. Fractions were analyzed by 8-16% SDS-PAGE gel electrophoresis as described above, which revealed the Cel6A cellobiohydrolase eluted at the very end of the gradient (approximately 50 mM ammonium sulfate).

The A. aculeatus Cel6A cellobiohydrolase was greater than 90% pure as judged by SDS-PAGE. Protein concentrations were determined using a BCA Protein Assay Kit (Thermo Fisher Scientific, Waltham, Mass., USA) in which bovine serum albumin was used as a protein standard.

### Example 3

### Effect of Aspergillus aculeatus Family 6 Cellobiohydrolase on PCS Hydrolysis

Corn stover was pretreated at the U.S. Department of Energy National Renewable Energy Laboratory (NREL) using 1.4 wt % sulfuric acid at 165° C. and 107 psi for 8 minutes. The water-insoluble solids in the pretreated corn stover contained 57.5% cellulose, 4.6% hemicellulose and 28.4% lignin. Cellulose and hemicellulose were determined by a two-stage sulfuric acid hydrolysis with subsequent analysis of sugars by high performance liquid chromatography using NREL Standard Analytical Procedure #002. Lignin was determined gravimetrically after hydrolyzing the cellulose and hemicellulose fractions with sulfuric acid using NREL Standard Analytical Procedure #003.

The pretreated corn stover was milled and washed with water prior to use. Milled, washed pretreated corn stover (initial dry weight 32.35%) was prepared by milling in a Cosmos ICMG 40 wet multi-utility grinder (EssEmm Corporation, Tamil Nadu, India), and subsequently washing repeatedly with deionized water and decanting off the supernatant fraction. The dry weight of the milled, water-washed pretreated corn stover was found to be 7.114%.

A. aculeatus cellobiohydrolase was evaluated for its ability to enhance the hydrolysis of PCS by a Trichoderma reesei cellulolytic protein composition (Trichoderma reesei broth expressing Thermoascus aurantiacus GH61A and Aspergillus oryzae beta-glucosidase fusion; PCT/US2008/065417).

The hydrolysis of PCS was conducted using 2.2 ml deep-well plates (Axygen, Union City, Calif., USA) in a total reaction volume of 1.0 ml. The hydrolysis was performed with 50 mg of PCS per ml of 50 mM sodium acetate pH 5.0 buffer containing 1 mM manganese sulfate and a fixed protein loading of 2 mg of the T. reesei cellulolytic protein preparation per gram of cellulose or a 20% replacement (by protein) of the T. reesei cellulolytic protein preparation with A. aculeatus cellobiohydrolase enzyme (1.6 mg of the T. reesei cellulolytic protein composition per g of cellulose and 0.4 mg of each enzyme per g of cellulose). Hydrolysis assays were performed in triplicate for 72 hours at 50° C. Following hydrolysis, samples were filtered with a 0.45 μm Multiscreen 96-well filter plate (Millipore, Bedford, Mass., USA) and filtrates analyzed for sugar content as described below.

When not used immediately, filtered sugary aliquots were frozen at −20° C. Sugar concentrations of samples diluted in 0.005 M H₂SO₄ were measured after elution by 0.005 M H₂SO₄ with 0.05% w/w benzoic acid at a flow rate of 0.6 ml per minute from a 4.6×250 mm AMINEX® HPX-87H column (Bio-Rad Laboratories, Inc., Hercules, Calif., USA) at 65° C. with quantitation by integration of the glucose and cellobiose signal from refractive index detection (CHEMSTATION®, AGILENT® 1100 HPLC, Agilent Technologies, Santa Clara, Calif., USA) calibrated by pure sugar samples. The resultant equivalents were used to calculate the percentage of cellulose conversion for each reaction.

The degree of cellulose conversion was calculated using the following equation: % conversion=[glucose concentration+1.053×(cellobiose concentration)]/[(glucose concentration+1.053×(cellobiose concentration) in a limit digest]. The 1.053 factor for cellobiose takes into account the increase in mass when cellobiose is converted to glucose. Fifty mg of the T. reesei cellulolytic protein preparation per g of cellulose was used for the limit digest.

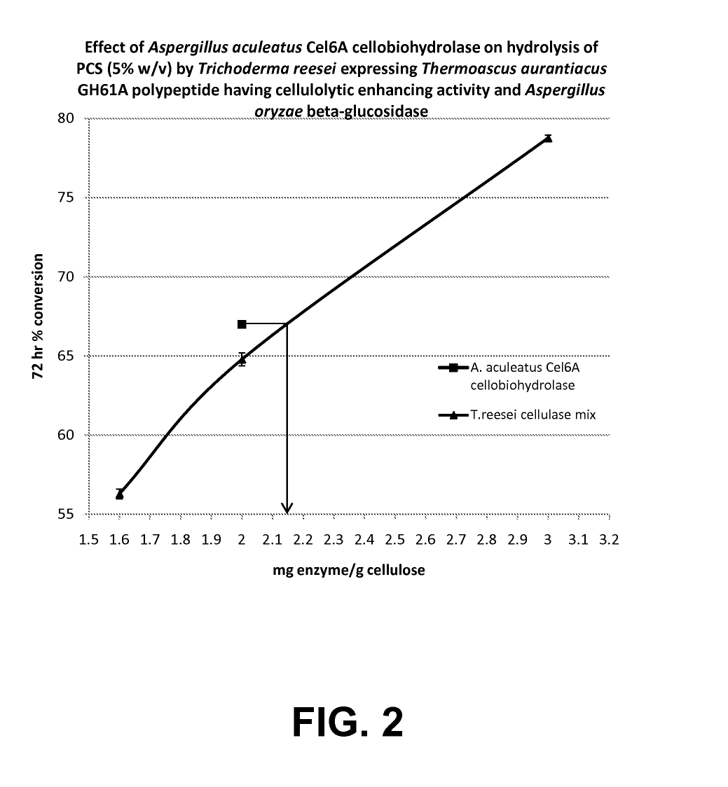

The results shown in FIG. 2 demonstrated that a 20% replacement (by protein) of the T. reesei cellulolytic protein preparation (loaded at 2 mg per g of cellulose) with A. aculeatus cellobiohydrolase improved the 72 hour hydrolysis yield by 3.4%. Alternatively, the percent conversion with a 20% replacement of a T. reesei cellulolytic protein preparation (loaded at 2 mg per g of cellulose) with the A. aculeatus Cel6A cellobiohydrolase was equivalent to a loading of 2.15 mg of the T. reesei cellulolytic protein preparation per g of cellulose (a 1.08-fold improvement).

### Example 4

### Identification of an Aspergillus aculeatus Family 6 cellobiohydrolase

In-Gel Digestion of Polypeptides for Peptide Sequencing.

A MULTIPROBE® II Liquid Handling Robot (PerkinElmer Life and Analytical Sciences, Boston, Mass., USA) was used to perform the in-gel digestions. The 70 kDa protein gel band described in Example 2 was excised with a razor blade and reduced with 50 μl of 10 mM dithiothreitol (DTT) in 100 mM ammonium bicarbonate pH 8.0 for 30 minutes. Following reduction, the gel piece was alkylated with 50 μl of 55 mM iodoacetamide in 100 mM ammonium bicarbonate pH 8.0 for 20 minutes. The dried gel piece was allowed to swell in 25 μl of a trypsin digestion solution containing 6 ng of sequencing grade trypsin (Promega, Madison, Wis., USA) per μl of 50 mM ammonium bicarbonate pH 8 for 30 minutes at room temperature, followed by an 8 hour digestion at 40° C. Each of the reaction steps described above was followed by numerous washes and pre-washes with the appropriate solutions following the manufacturer's standard protocol. Fifty μl of acetonitrile was used to de-hydrate the gel piece between reactions and the gel piece was air dried between steps. Peptides were extracted twice with 1% formic acid/2% acetonitrile in HPLC grade water for 30 minutes. Peptide extraction solutions were transferred to a 96 well skirted PCR type plate (ABGene, Rochester, N.Y., USA) that had been cooled to 10-15° C. and covered with a 96-well plate lid (PerkinElmer Life and Analytical Sciences, Boston, Mass., USA) to prevent evaporation. Plates were further stored at 4° C. until mass spectrometry analysis was performed.

Protein Identification.

For de novo peptide sequencing by tandem mass spectrometry, a Q-TOFMICRO™ (Waters Micromass MS Technologies, Milford, Mass., USA), a hybrid orthogonal quadrupole time-of-flight mass spectrometer, was used for LC/MS/MS analysis. The Q-TOF MICRO™ is fully microprocessor controlled using MASSLYNX™ software version 4.1 (Waters Micromass MS Technologies, Milford, Mass., USA). The Q-TOF MICRO™ was fitted with a NANOACQUITY HPLC® (Waters Corp, Milford, Mass., USA) for concentrating and desalting samples. Samples were loaded onto a trapping column (180 μm ID×20 mm, 5 μm SYMMETRY® C18) (Waters Corp, Milford, Mass., USA) fitted in the injection loop and washed with 0.1% formic acid in water at 15 μl per minute for 1 minute using the binary solvent manager pump. Peptides were separated on a 100 μm ID×100 mm, C18, 1.7 μm, BEH130™ C18 nanoflow fused capillary column (Waters Corp, Milford, Mass., USA) at a flow rate of 400 nl per minute. A step elution gradient of 1% to 85% acetonitrile in 0.1% formic acid was applied over a 30 minute interval. The column eluent was monitored at 214 nm and introduced into the Q-TOF MICRO™ through an electrospray ion source fitted with the nanospray interface.

Data was acquired in survey scan mode from a mass range of m/z 400 to 1990 with switching criteria for MS to MS/MS to include an ion intensity of greater than 10.0 counts per second and charge states of +2, +3, and +4. Analysis spectra of up to 6 co-eluting species with a scan time of 1.9 seconds and inter-scan time of 0.1 seconds could be obtained. A cone voltage of 45 volts was typically used and the collision energy was programmed to be varied according to the mass and charge state of the eluting peptide and in the range of 10-60 volts. The acquired spectra were combined, smoothed, and centered in an automated fashion and a peak list generated. The peak list was searched against selected databases using PROTEINLYNX GLOBAL SERVER™ 2.3 software (Waters Micromass MS Technologies, Milford, Mass., USA) and PEAKS Studio version 4.5 (SP1) (Bioinformatic Solutions Inc., Waterloo, Ontario, Canada). Results from the PROTEINLYNX GLOBAL SERVER™ and PEAKS Studio searches were evaluated and un-identified proteins were analyzed further by evaluating the MS/MS spectrums of each ion of interest and de novo sequence was determined by identifying the y and b ion series and matching mass differences to the appropriate amino acid.

Peptide sequences were obtained from several multiple charged ions for the in-gel digested 70 kDa polypeptide gel band. A doubly charged tryptic peptide ion of 404.233 m/z sequence was determined to be Phe-[Ile/Leu]-Val-Asp-Thr-Gly-Arg (amino acids 370 to 376 of SEQ ID NO: 2). Another doubly charged tryptic peptide ion of 419.2206 m/z sequence was determined to be Ala-Tyr-[Ile/Leu]-Asp-Ser-[Ile/Leu]-Arg (amino acids 221 to 227 of SEQ ID NO: 2). Another doubly charged tryptic peptide ion of 486.313 m/z sequence was determined to be [Ile/Leu]-Val-Thr-Asn-[Ile/Leu]-Asn-Val-Ala-Lys (amino acids 250 to 258 of SEQ ID NO: 2). Another doubly charged tryptic peptide ion of 514.793 m/z sequence was determined to be Ala-Asn-[Ile/Leu]-Tyr-Ala-Ser-Val-Tyr-Lys (amino acids 304 to 312 of SEQ ID NO: 2). Another doubly charged tryptic peptide ion of 575.817 m/z sequence was determined to be Ser-[Ile/Leu]-Ala-Asn-Asn-Gly-Val-Ala-Asn-Tyr-Lys (amino acids 210 to 220 of SEQ ID NO: 2). Another double charged tryptic peptide of 666.3743 was determined to be Val-Pro-Ser-Phe-Val-Trp-Leu-Asp-Val-Ala-Ala-Lys (amino acids 152 to 163 of SEQ ID NO:2). Another doubly charged tryptic peptide ion of 669.881 m/z a was determined to be Val-Pro-Thr-Met-Ala-Thr-Tyr-[Ile/Leu]-Ala-Asp-[Ile/Leu]-Lys (amino acids 164 to 175 of SEQ ID NO: 2). [Ile/Leu] could not be distinguished because they have equivalent masses.

### Example 5

### Preparation of Aspergillus aculeatus Strain NN000525 Mycelia for cDNA Library Production

A. aculeatus strain NN000525 was inoculated onto a PDA plate and incubated for 4 days at 37° C. in the darkness. Several mycelia-PDA plugs were inoculated into 500 ml shake flasks containing 150 ml of NNCYP-PCS medium. The flasks were incubated for 4 days at 26° C. with shaking at 120 rpm. The mycelia from the solid media were collected and frozen in liquid nitrogen and then stored in a −80° C. freezer until use.

### Example 6

### Aspergillus aculeatus Strain NN000525 RNA Isolation

The frozen mycelia were transferred into a liquid nitrogen prechilled mortar and pestle and ground to a fine powder with a small amount of baked quartz sand. Total RNA was prepared from the powdered mycelia by extraction with TRIZOL® LS (Invitrogen Corp., Carlsbad, Calif., USA) followed by triple extraction with chloroform and precipitation with 0.7 v/v isopropanol. The total RNA pellet was redissolved in RNAase free water and stored in a −80° C. freezer until use.

### Example 7

### Construction of Aspergillus aculeatus Strain NN000525 cDNA

Double stranded cDNA was synthesized using a SMART™ PCR cDNA Synthesis Kit (Clontech, Saint-Germain-en-Laye, France) according to the manufacturer's LD PCR cDNA amplification protocol.

### Example 8

### Isolation of the cDNA Encoding Aspergillus aculeatus Strain NN000525 GH6 Polypeptide

PCR was used to amplify a fragment of the cDNA containing the 5′ end using the SMART™ II A oligonucleotide (Clontech, Saint-Germain-en-Laye, France) and the following degenerate primers (TAG Copenhagen, Denmark):

Primer #578:
(SEQ ID NO: 135)
5′-GAGCAGTCTCGGTCGGGNADRTTRTA-3′
Primer #580:
(SEQ ID NO: 136)
5′-GCCGTCGGACTCGCCNCCNGGYTT-3′

The amplification reaction was composed of 1 μl of Aspergillus aculeatus strain NN000525 SMART™ cDNA, 12.5 μl of 2× REDDYMIX™ PCR Buffer (Thermo Fisher Scientific Inc., Waltham, Mass., USA), 1 μl of SMART™ II A oligonucleotide, 9.5 μl of H₂O, and 1 μl of a 5 μM solution of either primer #578 or primer #580. The amplification reactions were incubated in a PTC-200 DNA ENGINE™ Thermal Cycler (MJ Research Inc., Waltham, Mass., USA) programmed for 1 cycle at 94° C. for 2 minutes; and 35 cycles each at 94° C. for 15 seconds and 60° C. for 1 minute.

A 0.9 kb PCR reaction product and a 1.3 kb PCR reaction product were isolated by 1% agarose gel electrophoresis using TAE buffer (40 mM Tris base-20 mM sodium acetate-1 mM disodium EDTA) and staining with SYBR® Safe DNA gel stain (Invitrogen Corp., Carlsbad, Calif., USA). The DNA bands were visualized with the aid of an EAGLE EYE® Imaging System (Stratagene, La Jolla, Calif., USA) and a DARKREADER® Transilluminator (Clare Chemical Research, Dolores, Colo., USA). The 0.9 and 1.3 kb DNA bands were excised from the gels and purified using a GFX® PCR DNA and Gel Band Purification Kit (GE Healthcare Life Sciences, Piscataway, N.J., USA) according to the manufacturer's instructions. The 0.9 kb band was sequenced with the #578 primer and the 1.3 kb fragment was sequenced using the #580 primer.

The 3′ end of the cDNA was amplified using the CDSIII oligonucleotide (Clontech, Saint-Germain-en-Laye, France) together with either of the following primers (TAG Copenhagen, Denmark):

Primer #601:
(SEQ ID NO: 137)
5′-CTCCTACACCCAGGGCAACA-3′
Primer #602:
(SEQ ID NO: 138)
5′-CGATTGGTGCAACGTCATCA-3′

The amplification reactions were composed of 1 μl of A. aculeatus strain NN000525 SMART cDNA, 12.5 μl of 2× REDDYMIX™ PCR Buffer, 9.5 μl of H₂O, and 1 μl of a 5 μM solution of primer #601 or primer #602. The amplification reactions were incubated in a PTC-200 DNA ENGINE™ Thermal Cycler programmed for 1 cycle at 94° C. for 2 minutes; and 35 cycles each at 94° C. for 15 seconds and 60° C. for 1 minute.

A 0.6 kb PCR reaction product and a 0.4 kbp PCR reaction product were isolated by 1% agarose gel electrophoresis using TAE buffer and staining with SYBR® Safe DNA gel stain. The DNA bands were visualized with the aid of an EAGLE EYE® Imaging System and a DARKREADER® Transilluminator. The 0.6 kb and a 0.4 kb DNA bands were excised from the gels and purified using a GFX® PCR DNA and Gel Band Purification Kit according to the manufacturer's instructions. Both fragments were sequenced using primer #602.

### Example 9

### Characterization of the Aspergillus aculeatus Strain NN000525 cDNA Sequence Encoding a Family GH6 Polypeptide Having Cellobiohydrolase Activity

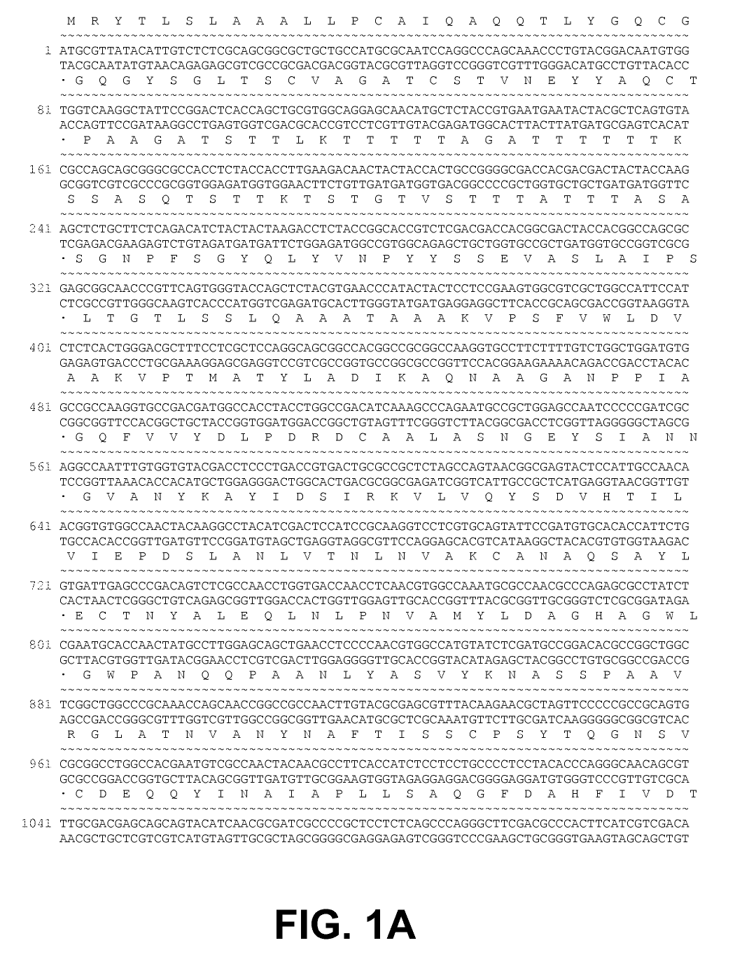

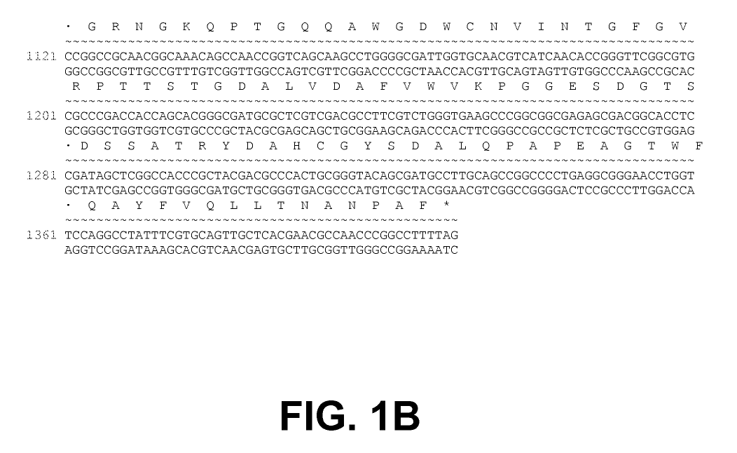

The nucleotide sequence (SEQ ID NO: 1) and deduced amino acid sequence (SEQ ID NO: 2) of the cDNA encoding the A. aculeatus GH6 polypeptide having cellobiohydrolase activity are shown in FIGS. 1A and 1B. The open reading frame is 1407 bp including the stop codon, and encodes a polypeptide of 469 amino acids. The % G+C content of the full-length coding sequence and the mature coding sequence is 61.9% and 62.0%, respectively. Using the SignalP software program (Nielsen et al., 1997, Protein Engineering 10:1-6), a signal peptide of 18 residues was predicted. The predicted mature protein contains 451 amino acids with a molecular mass of 47 kDa.

Analysis of the deduced amino acid sequence of the GH6 polypeptide having cellobiohydrolase activity with the Interproscan program (Mulder et al., 2007, Nucleic Acids Res. 35: D224-D228) showed that the GH6 polypeptide contained the sequence signature of glycoside hydrolase family 6 (InterPro accession IPR001524). This sequence signature was found from approximately residues 90 to 451 of the mature polypeptide (Pfam accession PF01341). The Interproscan program analysis also revealed a CBM 1 cellulose binding domain (InterPro accession IPR000254). This sequence signature was found from approximately residues 4 to 37 of the mature polypeptide (Pfam accession PF01341).

A comparative pairwise global alignment of amino acid sequences was determined using the Needleman-Wunsch algorithm (Needleman and Wunsch, 1970, J. Mol. Biol. 48: 443-453) as implemented in the Needle program of EMBOSS with gap open penalty of 10, gap extension penalty of 0.5, and the EBLOSUM62 matrix. The alignment showed that the deduced amino acid sequence of the A. aculeatus GH6 mature polypeptide shared 98% identity (excluding gaps) to the deduced amino acid sequence of a fungal Family 6 glycoside hydrolase protein (GeneSeqP accession number ASR94299).

### Example 10

### Cloning of the Aspergillus aculeatus Strain NN000525 GH6 Polypeptide Encoding cDNA

Based on the cDNA sequence, oligonucleotide primers, shown below, were designed to amplify the GH6 gene from cDNA of A. aculeatus strain NN000525.

Primer #609:
(SEQ ID NO: 139)
5′-TAAGAATTCACCATGCGTTATACATTGTCTCTCGCA3′
Primer #608:
(SEQ ID NO: 140)
5′-TATGCGGCCGCYTARAANGCNGGRTTNGCRTT-3′

The amplification reaction was composed of 1 μl of Aspergillus aculeatus strain NN000525 SMART cDNA, 12.5 μl of 2× REDDYMIX™ PCR Buffer, 1 μl of 5 μM primer #609, 1 μl of 5 μM primer #608, and 9.5 μl of H₂O. The amplification reaction was incubated in a PTC-200 DNA ENGINE™ Thermal Cycler programmed for 1 cycle at 94° C. for 2 minutes; and 35 cycles each at 94° C. for 15 seconds and 60° C. for 1.5 minutes.

A 1.4 kb PCR reaction product was isolated by 1% agarose gel electrophoresis using TAE buffer and staining with SYBR® Safe DNA gel stain. The DNA band was visualized with the aid of an EAGLE EYE® Imaging System and a DARKREADER® Transilluminator. The 1.4 kb DNA band was excised from the gel and purified using a GFX® PCR DNA and Gel Band Purification Kit according to the manufacturer's instructions.

The 1.4 kb fragment was cleaved with Eco RI and Not I and purified using a GFX® PCR DNA and Gel Band Purification Kit according to the manufacturer's instructions.

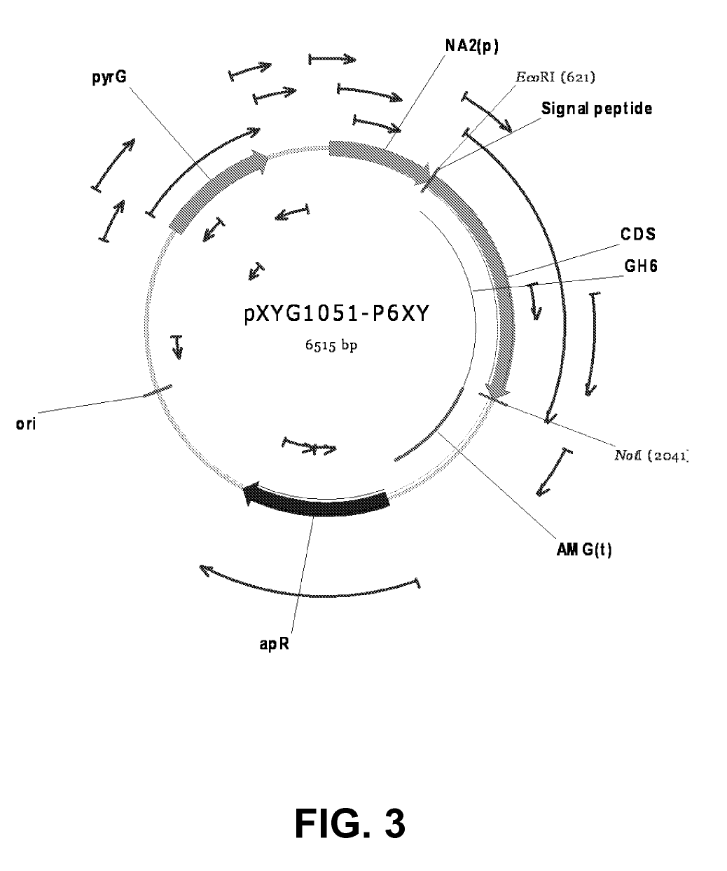

The cleaved 1.4 kb fragment was then directionally cloned by ligation into Eco RI-Not I cleaved pXYG1051 (WO 2005/080559) using T4 ligase (Promega, Madison, Wis., USA) according to the manufacturer's instructions. The ligation mixture was transformed into E. coli TOP10F competent cells (Invitrogen Corp., Carlsbad, Calif., USA) according to the manufacturer's instructions. The transformation mixture was plated onto LB plates supplemented with 100 μg of ampicillin per ml. Plasmid minipreps were prepared from several transformants and sequenced. One plasmid with the correct Aspergillus aculeatus GH6 coding sequence was chosen. The plasmid was designated pXYG1051-P6XY (FIG. 3). The expression vector pXYG1051 contains the same neutral amylase II (NA2) promoter derived from Aspergillus niger, and terminator elements as pCaHj483 (disclosed in Example 4 of WO 98/00529). Furthermore pXYG1051 has pUC18 derived sequences for selection and propagation in E. coli, and pDSY82 (disclosed in Example 4 of U.S. Pat. No. 5,958,727) derived sequences for selection and expression in Aspergillus facilitated by the pyrG gene of Aspergillus oryzae, which encodes orotidine decarboxylase and is used to complement a pyrG mutant Aspergillus strain.

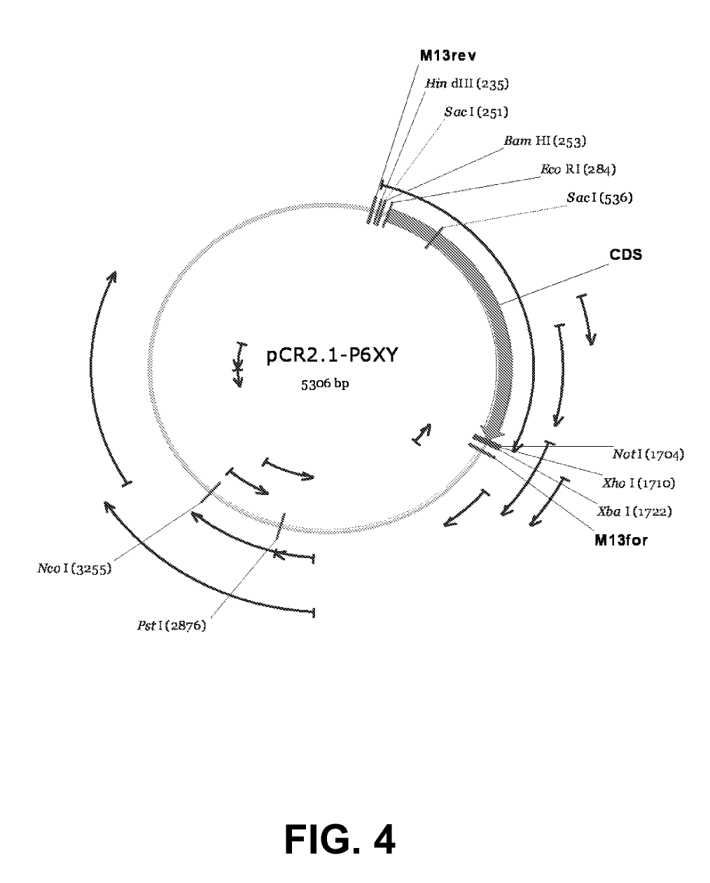

The 1.4 kb fragment PCR amplified by primers #609 and #608 was also cloned by ligation into pCR®2.1 (Invitrogen, Carlsbad, Calif., USA) digested with Eco RI and Not I using standard molecular biology techniques to yield pCR2.1-P6XY (FIG. 4). The Aspergillus aculeatus GH6 polypeptide gene insert in pCR2.1-P6XY was determined by Sanger sequencing to encode the same polypeptide sequence as in pXYG1051-P6XY, but varied at several positions (SEQ ID NO: 141) corresponding to the wobble bases of primer #608. These changes can easily be corrected by site-directed mutagenesis. E. coli NN059164 containing pCR2.1-P6XY was deposited with the Deutsche Sammlung von Mikroorganismen and Zellkulturen GmbH (DSM) on Oct. 1, 2009 and assigned accession number DSM 22994.

### Example 11

### Production of Recombinant Aspergillus aculeatus GH6 Polypeptide Having Cellobiohydrolase Activity in Aspergillus oryzae

The expression plasmid pXYG1051-P6XY was transformed into Aspergillus oryzae JaL355 as described in WO 98/00529. Transformants were purified on selection plates through single conidia prior to sporulating them on PDA plates. Production of the Aspergillus aculeatus GH6 polypeptide by the transformants was analyzed from culture supernatants of 1 ml 96 deep well stationary cultivations at 26° C. in YP medium with 2% maltodextrin. Expression was verified on NUPAGE® 10% Bis-Tris SDS-PAGE (Invitrogen, Carlsbad, Calif., USA) by Coomassie blue staining. One transformant was selected for further work and designated Aspergillus oryzae 86.10.

For larger scale production, Aspergillus oryzae 86.10 spores were spread onto a PDA plate and incubated for five days at 37° C. The confluent spore plate was washed twice with 5 ml of 0.01% TWEEN® 20 to maximize the number of spores collected. The spore suspension was then used to inoculate twenty-five 500 ml flasks containing 100 ml of YPM medium. The culture was incubated at 30° C. with constant shaking at 85 rpm. At day four post-inoculation, the culture broth was collected by filtration through a triple layer of Whatman glass microfiber filters of 1.6 μm, 1.2 μm, and 0.7 μm. Fresh culture broth from this transformant produced a band of GH6 protein of approximately 70 kDa.

### Example 12

### Purification of Recombinant Aspergillus aculeatus Cel6A Cellobiohydrolase

One liter of harvested broth (Example 11) was sterile filtered using a 0.22 μm polyethersulfone membrane (Millipore, Bedford, Mass., USA). Ammonium sulfate was added to the filtered broth to 2 M ammonium sulfate as a final concentration and applied to a 70 ml PHENYL SEPHAROSE™ Fast Flow column (GE Healthcare, Piscataway, N.J., USA). The column was washed with 3 column volumes of 2 M ammonium sulfate and then 5 column volumes of 1 M ammonium sulfate. Bound proteins were eluted with a decreasing salt gradient (2 column volumes) of 1 M ammonium sulfate to 0 M ammonium sulfate in 20 mM HEPES pH 7.0. Fractions were analyzed by SDS-PAGE using 4-20% NUPAGE® Bis/Tris, SDS-PAGE gels (Invitrogen Corporation, Carlsbad, Calif., USA) stained with INSTANTBLUE™ Stain (Expedeon Protein Solutions, Cambridge, UK). The eluate fractions containing A. aculeatus Cel6A cellobiohydrolase as judged by the presence of a 65-70 kDa band corresponding to the apparent molecular weight of the Cel6A cellobiohydrolase were pooled and desalted (500 ml SEPHADEX™ G-25 Medium column, GE Healthcare, Piscataway, N.J., USA) into 20 mM HEPES pH 7.5.

The desalted material was applied to a 20 ml SOURCE™ 15Q column (GE Healthcare, Piscataway, N.J., USA) equilibrated with 20 mM HEPES pH 7.5. Bound proteins were eluted with a salt gradient (10 column volumes) from 0 M NaCl to 500 mM NaCl in 50 mM HEPES pH 7.5. Flow through and eluate fractions were examined by SDS-PAGE using 4-20% NUPAGE® Bis/Tris, SDS-PAGE gels stained with INSTANTBLUE™ Stain. The flow-through fraction contained A. aculeatus Cel6A cellobiohydrolase and was concentrated (VIVASPIN™ 20, 10 kDa membrane, Sartorius Stedim Biotech S.A., Aubagne, France).

The A. aculeatus Cel6A cellobiohydrolase was greater than 90% pure as judged by SDS-PAGE. Protein concentrations were determined by the absorbance at 280 nm using an extinction coefficient of 1.54 (ml)(cm⁻¹)(mg⁻¹).

### Example 13

### Effect of Recombinant Aspergillus aculeatus Cel6A Cellobiohydrolase on PCS Hydrolysis

Corn stover was pretreated at the U.S. Department of Energy National Renewable Energy Laboratory (NREL) using 0.048 g sulfuric acid/g dry biomass at 190° C. and 25% w/w dry solids for approximately 1 minute. The water-insoluble solids in the pretreated corn stover contained 52% cellulose, 3.6% hemicellulose and 29.8% lignin. Cellulose and hemicellulose were determined by a two-stage sulfuric acid hydrolysis with subsequent analysis of sugars by high performance liquid chromatography using NREL Standard Analytical Procedure #002. Lignin was determined gravimetrically after hydrolyzing the cellulose and hemicellulose fractions with sulfuric acid using NREL Standard Analytical Procedure #003. The pretreated corn stover was ground using a Multi Utility Grinder (iNNo Concepts Inc., Roswell, Ga., USA) and sieved through a Sieve Shaker AS200 equipped with a 450 μm screen (Retsch, Inc. Newtown, Pa., USA) and designated herein as GS-PCS.

The recombinant A. aculeatus cellobiohydrolase purified according to Example 12 was evaluated for its ability to enhance the hydrolysis of GS-PCS by CELLIC™ CTec (a cellulolytic protein composition available from Novozymes A/S, Bagsvaerd, Denmark). The protein concentration was determined by a BCA reagent Kit (Pierce, Rockford, Ill., USA).

Hydrolysis of GS-PCS was performed in 96 well plates in a total reaction volume of 1.0 ml. The hydrolysis was performed with 50 mg of GS-PCS per ml of 50 mM sodium acetate pH 5.0 buffer containing 1 mM manganese sulfate and 3 mg of CELLIC™ CTec per gram of cellulose and 0.6 mg of the A. aculeatus cellobiohydrolase per gram of cellulose for a total loading of 3.6 mg protein per g cellulose. The hydrolysis assays were performed in duplicate for 72 hours at 50° C. Following hydrolysis, samples were filtered with a 0.45 μm Multiscreen 96-well filter plate (Millipore, Bedford, Mass., USA), diluted 2-fold in 5 mM H₂SO₄, and analyzed by HPLC as described below. Sugar concentrations were measured after elution by 0.005 M H₂SO₄ with 0.05% w/w benzoic acid at a flow rate of 0.6 ml per minute from a 4.6×250 mm AMINEX® HPX-87H column at 65° C. using refractive index detection. Hydrolysis data are presented as % of total cellulose converted to glucose. The degree of cellulose conversion to reducing sugar was calculated using the following equation:

Conversion_{(%)}=RS_{(mg/ml)}*100*162/(Cellulose_{(mg/ml)}*180)=RS_{(mg/ml)}*100/(Cellulose_{(mg/ml)}*1.111)

In this equation, RS is the concentration of reducing sugar in solution measured in glucose equivalents (mg/ml), and the factor 1.111 reflects the weight gain in converting cellulose to glucose.

The results demonstrated that the A. aculeatus GH6 cellobiohydrolase at 0.6 mg/g cellulose and CELLIC™ CTec at 3 mg/g cellulose yielded a cellulose conversion of 64.7% after 72 hours, while CELLIC™ CTec alone at 3 mg/g cellulose yielded a cellulose conversion of 58.6%, CELLIC™ CTec alone at 3.6 mg/g cellulose yielded a cellulose conversion of 66.8%, and the A. aculeatus GH6 cellobiohydrolase alone at 0.6 mg/g cellulose yielded a cellulose conversion of 1%. The A. aculeatus GH6 cellobiohydrolase had synergistic effect on CELLIC™ CTec in GS-PCS hydrolysis at 50° C., pH 5.0.

### Example 13

### Characterization of Aspergillus aculeatus GH6 Cellobiohydrolase

Specific activity: Phosphoric acid swollen cellulose (PASC) was dissolved in 50 mM sodium acetate pH 5 with 0.01% TWEEN® 20 at 2.1 g per liter. Enzyme was diluted in the same buffer to a range of dilutions. To 190 μl of the PASC solution was added 10 μl of each enzyme dilution. The reaction was incubated at 50° C. for 30 minutes before the reaction was stopped with 50 μl of 0.5 M sodium hydroxide followed by centrifugation at 800×g for 5 minutes. Supernatant was removed and reducing sugar was measured using p-hydroxybenzoic acid hydrazide (PHBAH) reagent according to Lever, 1973, Biochem. Med. 7:274-287. An enzyme control, reagent control and substrate control were included. The absorbance at 405 nm was measured for 4-nitrophenolate production. The specific activity of the A. aculeatus GH6 cellobiohydrolase on PASC was determined to be 1.6 IU/mg.

Thermostability: The A. aculeatus GH6 cellobiohydrolase was diluted in 50 mM sodium acetate pH 5 containing 0.01% TWEEN® 20 to 1 mg per ml, and then incubated at 50° C. for 3 days and 60° C. for 3 hours and 24 hours. The same sample was stored at 4° C. to serve as control. After incubation, the activity of the samples on PASC was measured as described above using one enzyme loading which gave less than 5% conversion. The activity of the sample at 4° C. was normalized to 100%, and the activities of the other samples at other incubation conditions were compared to the 4° C. activity. The thermostability of the A. aculeatus GH6 cellobiohydrolase is shown below.

| Incubation conditions | Residual % Activity |
|---|---|
| 4° C. | 100% |
| 50° C., 72 hr | 100% |
| 60° C., 3 hr | 52% |
| 60° C., 24 hr | 0% |

pH profile: The pH activity profile of the A. aculeatus GH6 cellobiohydrolase was determined using the same protocol described above, except the cellobiohydrolase was incubated at five different pHs (4, 5, 6, 7, and 8) and one enzyme loading was used, which yielded less than 5% conversion. Britton Robinson buffer (100 mM) was used as the buffer system. The 100 mM Britton Robinson buffer was titrated to a various pH values in the range of 4-7 using 5 M sodium hydroxide and then diluted to 40 mM with deionized water. PASC was prepared in the same buffers. Cellobiohydrolase activity was measured at 50° C. The highest activity was normalized to be 100%, and activities at other pH values were compared to the highest activity and expressed in % activity. The pH profile of the A. aculeatus GH6 cellobiohydrolase is shown below.

| | Relative % |
|---|---|
| pH | Activity |
| 4.0 | 43% |
| 5.0 | 93% |
| 6.0 | 100% |
| 7.0 | 90% |
| 8.0 | 70% |

### Deposit of Biological Material

The following biological material has been deposited under the terms of the Budapest Treaty with the Deutsche Sammlung von Mikroorganismen and Zellkulturen GmbH (DSM), Mascheroder Weg 1 B, D-38124 Braunschweig, Germany, and given the following accession number:

| Deposit | Accession Number | Date of Deposit |
|---|---|---|
| E. coli | DSM 22994 | Oct. 1, 2009 |

The strain has been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

The present invention is further described by the following numbered paragraphs:

[1] An isolated polypeptide having cellobiohydrolase activity, selected from the group consisting of: (a) a polypeptide comprising an amino acid sequence having at least 99% identity to the mature polypeptide of SEQ ID NO: 2; (b) a polypeptide encoded by a polynucleotide comprising a nucleotide sequence having at least 99% identity to the mature polypeptide coding sequence of SEQ ID NO: 1; and (c) a polypeptide comprising the mature polypeptide of SEQ ID NO: 2, or a fragment thereof having cellobiohydrolase activity.

[2] The polypeptide of paragraph 1, comprising an amino acid sequence having at least 99% identity to the mature polypeptide of SEQ ID NO: 2.

[3] The polypeptide of paragraph 1, comprising or consisting of the amino acid sequence of SEQ ID NO: 2; or a fragment thereof having cellobiohydrolase activity.

[4] The polypeptide of paragraph 1, comprising or consisting of the amino acid sequence of SEQ ID NO: 2.

[5] The polypeptide of paragraph 1, comprising or consisting of the mature polypeptide of SEQ ID NO: 2.

[6] The polypeptide of paragraph 1, which is encoded by a polynucleotide comprising a nucleotide sequence having at least 99% identity to the mature polypeptide coding sequence of SEQ ID NO: 1

[7] The polypeptide of paragraph 1, which is encoded by a polynucleotide comprising or consisting of the nucleotide sequence of SEQ ID NO: 1; or a subsequence thereof encoding a fragment having cellobiohydrolase activity.

[8] The polypeptide of paragraph 7, which is encoded by a polynucleotide comprising or consisting of the nucleotide sequence of SEQ ID NO: 1.

[9] The polypeptide of paragraph 7, which is encoded by a polynucleotide comprising or consisting of the mature polypeptide coding sequence of SEQ ID NO: 1.

[10] The polypeptide of paragraph 1, which is encoded by the polynucleotide contained in plasmid pCR2.1-P6XY which is contained in E. coli DSM 22994.

[11] The polypeptide of any of paragraphs 1-12, wherein the mature polypeptide is amino acids 19 to 469 of SEQ ID NO: 2.

[12] The polypeptide of any of paragraphs 1-13, wherein the mature polypeptide coding sequence is nucleotides 55 to 1407 of SEQ ID NO: 1.

[13] An isolated polynucleotide comprising a nucleotide sequence that encodes the polypeptide of any of paragraphs 1-12.

[14] A nucleic acid construct comprising the polynucleotide of paragraph 13 operably linked to one or more (several) control sequences that direct the production of the polypeptide in an expression host.

[15] A recombinant expression vector comprising the polynucleotide of paragraph 13.

[16] A recombinant host cell comprising the polynucleotide of paragraph 13 operably linked to one or more (several) control sequences that direct the production of a polypeptide having cellobiohydrolase activity.

[17] A method of producing the polypeptide of any of paragraphs 1-12, comprising: (a) cultivating a cell, which in its wild-type form produces the polypeptide, under conditions conducive for production of the polypeptide; and (b) recovering the polypeptide.

[18] A method of producing the polypeptide of any of paragraphs 1-12, comprising: (a) cultivating a host cell comprising a nucleic acid construct comprising a polynucleotide encoding the polypeptide under conditions conducive for production of the polypeptide; and (b) recovering the polypeptide.

[19] A method of producing a mutant of a parent cell, comprising disrupting or deleting a polynucleotide encoding the polypeptide, or a portion thereof, of any of paragraphs 1-12, which results in the mutant producing less of the polypeptide than the parent cell.

[20] A mutant cell produced by the method of paragraph 19.

[21] The mutant cell of paragraph 20, further comprising a gene encoding a native or heterologous protein.

[22] A method of producing a protein, comprising: (a) cultivating the mutant cell of paragraph 21 under conditions conducive for production of the protein; and (b) recovering the protein.

[23] A method of producing the polypeptide of any of paragraphs 1-12, comprising: (a) cultivating a transgenic plant or a plant cell comprising a polynucleotide encoding the polypeptide under conditions conducive for production of the polypeptide; and (b) recovering the polypeptide.

[24] A transgenic plant, plant part or plant cell transformed with a polynucleotide encoding the polypeptide of any of paragraphs 1-12.

[25] A double-stranded inhibitory RNA (dsRNA) molecule comprising a subsequence of the polynucleotide of paragraph 13, wherein optionally the dsRNA is a siRNA or a miRNA molecule.

[26] The double-stranded inhibitory RNA (dsRNA) molecule of paragraph 25, which is about 15, 16, 17, 18, 19, 20, 21, 22, 23, 24, 25 or more duplex nucleotides in length.

[27] A method of inhibiting the expression of a polypeptide having cellobiohydrolase activity in a cell, comprising administering to the cell or expressing in the cell the double-stranded inhibitory RNA (dsRNA) molecule of paragraph 25 or 26.

[28] An isolated polynucleotide encoding a signal peptide comprising or consisting of amino acids 1 to 18 of SEQ ID NO: 2.

[29] A nucleic acid construct comprising a gene encoding a protein operably linked to the polynucleotide of paragraph 28, wherein the gene is foreign to the polynucleotide.

[30] A recombinant expression vector comprising the polynucleotide of paragraph 28.

[31] A recombinant host cell comprising the polynucleotide of paragraph 28.

[32] A method of producing a protein, comprising: (a) cultivating a recombinant host cell comprising a gene encoding a protein operably linked to the polynucleotide of paragraph 28, wherein the gene is foreign to the polynucleotide, under conditions conducive for production of the protein; and (b) recovering the protein.

[33] A composition comprising the polypeptide of any of paragraphs 1-12.

[34] The composition of paragraph 33, which further comprises one or more (several) enzymes selected from the group consisting of a cellulase, a GH61 polypeptide having cellulolytic enhancing activity, a hemicellulase, an expansin, an esterase, a laccase, a ligninolytic enzyme, a pectinase, a peroxidase, a protease, and a swollenin.

[35] A method for degrading or converting a cellulosic material, comprising: treating the cellulosic material with an enzyme composition in the presence of the polypeptide of any of paragraphs 1-12.

[36] The method of paragraph 35, wherein the cellulosic material is pretreated.

[37] The method of paragraph 35 or 36, further comprising recovering the degraded cellulosic material.

[38] The method of any of paragraphs 35-37, wherein the enzyme composition comprises one or more (several) enzymes selected from the group consisting of a cellulase, a GH61 polypeptide having cellulolytic enhancing activity, a hemicellulase, an expansin, an esterase, a laccase, a ligninolytic enzyme, a pectinase, a peroxidase, a protease, and a swollenin.

[39] The method of paragraph 38, wherein the cellulase one or more (several) enzymes selected from the group consisting of an endoglucanase, a cellobiohydrolase, and a beta-glucosidase.

[40] The method of paragraph 38, wherein the hemicellulase is one or more (several) enzymes selected from the group consisting of a xylanase, an acetyxylan esterase, a feruloyl esterase, an arabinofuranosidase, a xylosidase, and a glucuronidase.

[41] The method of any of paragraphs 35-40, wherein the degraded cellulosic material is a sugar.

[42] The method of paragraph 41, wherein the sugar is selected from the group consisting of glucose, xylose, mannose, galactose, and arabinose.

[43] A method for producing a fermentation product, comprising: (a) saccharifying a cellulosic material with an enzyme composition in the presence of the polypeptide of any of paragraphs 1-12; (b) fermenting the saccharified cellulosic material with one or more fermenting microorganisms to produce the fermentation product; and (c) recovering the fermentation product from the fermentation.

[44] The method of paragraph 43, wherein the cellulosic material is pretreated.

[45] The method of paragraph 43 or 44, wherein the enzyme composition comprises one or more (several) enzymes selected from the group consisting of a cellulase, a GH61 polypeptide having cellulolytic enhancing activity, a hemicellulase, an expansin, an esterase, a laccase, a ligninolytic enzyme, a pectinase, a peroxidase, a protease, and a swollenin.

[46] The method of paragraph 45, wherein the cellulase is one or more (several) enzymes selected from the group consisting of an endoglucanase, a cellobiohydrolase, and a beta-glucosidase.

[47] The method of paragraph 45, wherein the hemicellulase is one or more (several) enzymes selected from the group consisting of a xylanase, an acetyxylan esterase, a feruloyl esterase, an arabinofuranosidase, a xylosidase, and a glucuronidase.

[48] The method of any of paragraphs 43-47, wherein steps (a) and (b) are performed simultaneously in a simultaneous saccharification and fermentation.

[49] The method of any of paragraphs 43-48, wherein the fermentation product is an alcohol, an organic acid, a ketone, an amino acid, or a gas.

[50] A method of fermenting a cellulosic material, comprising: fermenting the cellulosic material with one or more fermenting microorganisms, wherein the cellulosic material is saccharified with an enzyme composition in the presence of the polypeptide of any of paragraphs 1-12.

[51] The method of paragraph 50, wherein the cellulosic material is pretreated before saccharification.

[52] The method of paragraph 50 or 51, wherein the enzyme composition comprises one or more (several) enzymes selected from the group consisting of a cellulase, a GH61 polypeptide having cellulolytic enhancing activity, a hemicellulase, an expansin, an esterase, a laccase, a ligninolytic enzyme, a pectinase, a peroxidase, a protease, and a swollenin.

[53] The method of paragraph 52, wherein the cellulase is one or more (several) enzymes selected from the group consisting of an endoglucanase, a cellobiohydrolase, and a beta-glucosidase.

[54] The method of paragraph 52, wherein the hemicellulase is one or more (several) enzymes selected from the group consisting of a xylanase, an acetyxylan esterase, a feruloyl esterase, an arabinofuranosidase, a xylosidase, and a glucuronidase.

[55] The method of any of paragraphs 50-54, wherein the fermenting of the cellulosic material produces a fermentation product.

[56] The method of any of paragraphs 55, further comprising recovering the fermentation product from the fermentation.

[57] The method of paragraph 55 or 56, wherein the fermentation product is an alcohol, an organic acid, a ketone, an amino acid, or a gas.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An isolated polypeptide having cellobiohydrolase activity, selected from the group consisting of:
(a) a polypeptide comprising an amino acid sequence having at least 99% identity to the mature polypeptide of SEQ ID NO: 2;
(b) a polypeptide encoded by a polynucleotide comprising a nucleotide sequence having at least 99% identity to the mature polypeptide coding sequence of SEQ ID NO: 1; and
(d) a polypeptide comprising the mature polypeptide of SEQ ID NO: 2.

2. The polypeptide of claim 1, which is encoded by the polynucleotide contained in plasmid pCR2.1-P6XY which is contained in E. coli DSM 22994.

3. An isolated polynucleotide comprising a nucleotide sequence that encodes the polypeptide of claim 1.

4. A recombinant host cell comprising the polynucleotide of claim 3 operably linked to one or more (several) control sequences that direct the production of a polypeptide having cellobiohydrolase activity.

5. A method of producing the polypeptide of claim 1, comprising: (a) cultivating a cell, which in its wild-type form produces the polypeptide, under conditions conducive for production of the polypeptide; and (b) recovering the polypeptide.

6. A method of producing the polypeptide of claim 1, comprising: (a) cultivating a host cell comprising a nucleic acid construct comprising a polynucleotide encoding the polypeptide under conditions conducive for production of the polypeptide; and (b) recovering the polypeptide.

7. A method of producing a mutant of a parent cell, comprising disrupting or deleting a polynucleotide encoding the polypeptide, or a portion thereof, of claim 1, which results in the mutant producing less of the polypeptide than the parent cell.

8. A method of producing the polypeptide of claim 1, comprising: (a) cultivating a transgenic plant or a plant cell comprising a polynucleotide encoding the polypeptide under conditions conducive for production of the polypeptide; and (b) recovering the polypeptide.

9. A transgenic plant, plant part or plant cell transformed with a polynucleotide encoding the polypeptide of claim 1.

10. A double-stranded inhibitory RNA (dsRNA) molecule comprising a subsequence of the polynucleotide of claim 3, wherein optionally the dsRNA is a siRNA or a miRNA molecule.

11. A method of inhibiting the expression of a polypeptide having cellobiohydrolase activity in a cell, comprising administering to the cell or expressing in the cell the double-stranded inhibitory RNA (dsRNA) molecule of claim 10.

12. An isolated polynucleotide encoding a signal peptide comprising or consisting of amino acids 1 to 18 of SEQ ID NO: 2.

13. A method of producing a protein, comprising: (a) cultivating a recombinant host cell comprising a gene encoding a protein operably linked to the polynucleotide of claim 12, wherein the gene is foreign to the polynucleotide, under conditions conducive for production of the protein; and (b) recovering the protein.

14. A composition comprising the polypeptide of claim 1.

15. A method for degrading or converting a cellulosic material, comprising: treating the cellulosic material with an enzyme composition in the presence of the polypeptide of claim 1 or 2.

16. The method of claim 15, further comprising recovering the degraded cellulosic material.

17. A method for producing a fermentation product, comprising:
(a) saccharifying a cellulosic material with an enzyme composition in the presence of the polypeptide of claim 1;
(b) fermenting the saccharified cellulosic material with one or more fermenting microorganisms to produce the fermentation product; and
(c) recovering the fermentation product from the fermentation.

18. A method of fermenting a cellulosic material, comprising: fermenting the cellulosic material with one or more fermenting microorganisms, wherein the cellulosic material is saccharified with an enzyme composition in the presence of the polypeptide of claim 1.

19. The method of claim 18, wherein the fermenting of the cellulosic material produces a fermentation product.

20. The method of any of claim 19, further comprising recovering the fermentation product from the fermentation.


SEQUENCE LISTING (141 sequences)

SEQ ID NO: 1 - DNA, 1410 bp, Aspergillus aculeatus
atgcgttatacattgtctctcgcagcggcgctgctgccatgcgcaatccaggcccagcaaaccctgtacggacaatgtggtggtcaaggctattccggactcaccagctgcgtggcaggagcaacatgctctaccgtgaatgaatactacgctcagtgtacgccagcagcgggcgccacctctaccaccttgaagacaactactaccactgccggggcgaccacgacgactactaccaagagctctgcttctcagacatctactactaagacctctaccggcaccgtctcgacgaccacggcgactaccacggccagcgcgagcggcaacccgttcagtgggtaccagctctacgtgaacccatactactcctccgaagtggcgtcgctggccattccatctctcactgggacgctttcctcgctccaggcagcggccacggccgcggccaaggtgccttcttttgtctggctggatgtggccgccaaggtgccgacgatggccacctacctggccgacatcaaagcccagaatgccgctggagccaatcccccgatcgcaggccaatttgtggtgtacgacctccctgaccgtgactgcgccgctctagccagtaacggcgagtactccattgccaacaacggtgtggccaactacaaggcctacatcgactccatccgcaaggtcctcgtgcagtattccgatgtgcacaccattctggtgattgagcccgacagtctcgccaacctggtgaccaacctcaacgtggccaaatgcgccaacgcccagagcgcctatctcgaatgcaccaactatgccttggagcagctgaacctccccaacgtggccatgtatctcgatgccggacacgccggctggctcggctggcccgcaaaccagcaaccggccgccaacttgtacgcgagcgtttacaagaacgctagttcccccgccgcagtgcgcggcctggccacgaatgtcgccaactacaacgccttcaccatctcctcctgcccctcctacacccagggcaacagcgtttgcgacgagcagcagtacatcaacgcgatcgccccgctcctctcagcccagggcttcgacgcccacttcatcgtcgacaccggccgcaacggcaaacagccaaccggtcagcaagcctggggcgattggtgcaacgtcatcaacaccgggttcggcgtgcgcccgaccaccagcacgggcgatgcgctcgtcgacgccttcgtctgggtgaagcccggcggcgagagcgacggcacctccgatagctcggccacccgctacgacgcccactgcgggtacagcgatgccttgcagccggcccctgaggcgggaacctggttccaggcctatttcgtgcagttgctcacgaacgccaacccggccttttag

SEQ ID NO: 2 - protein, 469 aa, Aspergillus aculeatus
MRYTLSLAAALLPCAIQAQQTLYGQCGGQGYSGLTSCVAGATCSTVNEYYAQCTPAAGATSTTLKTTTTTAGATTTTTTKSSASQTSTTKTSTGTVSTTTATTTASASGNPFSGYQLYVNPYYSSEVASLAIPSLTGTLSSLQAAATAAAKVPSFVWLDVAAKVPTMATYLADIKAQNAAGANPPIAGQFVVYDLPDRDCAALASNGEYSIANNGVANYKAYIDSIRKVLVQYSDVHTILVIEPDSLANLVTNLNVAKCANAQSAYLECTNYALEQLNLPNVAMYLDAGHAGWLGWPANQQPAANLYASVYKNASSPAAVRGLATNVANYNAFTISSCPSYTQGNSVCDEQQYINAIAPLLSAQGFDAHFIVDTGRNGKQPTGQQAWGDWCNVINTGFGVRPTTSTGDALVDAFVWVKPGGESDGTSDSSATRYDAHCGYSDALQPAPEAGTWFQAYFVQLLTNANPAF

SEQ ID NO: 3 - DNA, 1377 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctt

SEQ ID NO: 4 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 5 - DNA, 1254 bp, Trichoderma reesei
atgaacaagtccgtggctccattgctgcttgcagcgtccatactatatggcggcgccgtcgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactagcagtggtaactcatggacggacacatccttggtcagctcgtgtctcgcaagaaag

SEQ ID NO: 6 - protein, 418 aa, Trichoderma reesei
MNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTSSGNSWTDTSLVSSCLARK

SEQ ID NO: 7 - DNA, 702 bp, Trichoderma reesei
atgaagttccttcaagtcctccctgccctcataccggccgccctggcccaaaccagctgtgaccagtgggcaaccttcactggcaacggctacacagtcagcaacaacctttggggagcatcagccggctctggatttggctgcgtgacggcggtatcgctcagcggcggggcctcctggcacgcagactggcagtggtccggcggccagaacaacgtcaagtcgtaccagaactctcagattgccattccccagaagaggaccgtcaacagcatcagcagcatgcccaccactgccagctggagctacagcgggagcaacatccgcgctaatgttgcgtatgacttgttcaccgcagccaacccgaatcatgtcacgtactcgggagactacgaactcatgatctggcttggcaaatacggcgatattgggccgattgggtcctcacagggaacagtcaacgtcggtggccagagctggacgctctactatggctacaacggagccatgcaagtctattcctttgtggcccagaccaacactaccaactacagcggagatgtcaagaacttcttcaattatctccgagacaataaaggatacaacgctgcaggccaatatgttcttagctaccaatttggtaccgagcccttcacgggcagtggaactctgaacgtcgcatcctggaccgcatctatcaac

SEQ ID NO: 8 - protein, 234 aa, Trichoderma reesei
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 9 - DNA, 726 bp, Trichoderma reesei
atgaaggcaactctggttctcggctccctcattgtaggcgccgtttccgcgtacaaggccaccaccacgcgctactacgatgggcaggagggtgcttgcggatgcggctcgagctccggcgcattcccgtggcagctcggcatcggcaacggagtctacacggctgccggctcccaggctctcttcgacacggccggagcttcatggtgcggcgccggctgcggtaaatgctaccagctcacctcgacgggccaggcgccctgctccagctgcggcacgggcggtgctgctggccagagcatcatcgtcatggtgaccaacctgtgcccgaacaatgggaacgcgcagtggtgcccggtggtcggcggcaccaaccaatacggctacagctaccatttcgacatcatggcgcagaacgagatctttggagacaatgtcgtcgtcgactttgagcccattgcttgccccgggcaggctgcctctgactgggggacgtgcctctgcgtgggacagcaagagacggatcccacgcccgtcctcggcaacgacacgggctcaactcctcccgggagctcgccgccagcgacatcgtcgagtccgccgtctggcggcggccagcagacgctctatggccagtgtggaggtgccggctggacgggacctacgacgtgccaggccccagggacctgcaaggttcagaaccagtggtactcccagtgtcttcct

SEQ ID NO: 10 - protein, 242 aa, Trichoderma reesei
MKATLVLGSLIVGAVSAYKATTTRYYDGQEGACGCGSSSGAFPWQLGIGNGVYTAAGSQALFDTAGASWCGAGCGKCYQLTSTGQAPCSSCGTGGAAGQSIIVMVTNLCPNNGNAQWCPVVGGTNQYGYSYHFDIMAQNEIFGDNVVVDFEPIACPGQAASDWGTCLCVGQQETDPTPVLGNDTGSTPPGSSPPATSSSPPSGGGQQTLYGQCGGAGWTGPTTCQAPGTCKVQNQWYSQCLP

SEQ ID NO: 11 - DNA, 923 bp, Humicola insolens
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggcggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatcccctccagcagcaccagctctccggtcaaccagcctaccagcaccagcaccacgtccacctccaccacctcgagcccgccagtccagcctacgactcccagcggctgcactgctgagaggtgggctcagtgcggcggcaatggctggagcggctgcaccacctgcgtcgctggcagcacttgcacgaagattaatgactggtaccatcagtgcctgtagaattc

SEQ ID NO: 12 - protein, 305 aa, Humicola insolens
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 13 - DNA, 1188 bp, Myceliopthora thermophila
cgacttgaaacgccccaaatgaagtcctccatcctcgccagcgtcttcgccacgggcgccgtggctcaaagtggtccgtggcagcaatgtggtggcatcggatggcaaggatcgaccgactgtgtgtcgggctaccactgcgtctaccagaacgattggtacagccagtgcgtgcctggcgcggcgtcgacaacgctgcagacatcgaccacgtccaggcccaccgccaccagcaccgcccctccgtcgtccaccacctcgcctagcaagggcaagctgaagtggctcggcagcaacgagtcgggcgccgagttcggggagggcaattaccccggcctctggggcaagcacttcatcttcccgtcgacttcggcgattcagacgctcatcaatgatggatacaacatcttccggatcgacttctcgatggagcgtctggtgcccaaccagttgacgtcgtccttcgaccagggttacctccgcaacctgaccgaggtggtcaacttcgtgacgaacgcgggcaagtacgccgtcctggacccgcacaactacggccggtactacggcaacatcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaccatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcgagcctccttcgggcaccggctatgtcaactacaactcgatcttgaagaagtacttgccgtaa

SEQ ID NO: 14 - protein, 389 aa, Myceliopthora thermophila
MKSSILASVFATGAVAQSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDQGYLRNLTEVVNFVTNAGKYAVLDPHNYGRYYGNIITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSTIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 15 - DNA, 1232 bp, BASIDIOMYCETE CBS 495.95
ggatccacttagtaacggccgccagtgtgctggaaagcatgaagtctctcttcctgtcacttgtagcgaccgtcgcgctcagctcgccagtattctctgtcgcagtctgggggcaatgcggcggcattggcttcagcggaagcaccgtctgtgatgcaggcgccggctgtgtgaagctcaacgactattactctcaatgccaacccggcgctcccactgctacatccgcggcgccaagtagcaacgcaccgtccggcacttcgacggcctcggccccctcctccagcctttgctctggcagccgcacgccgttccagttcttcggtgtcaacgaatccggcgcggagttcggcaacctgaacatccccggtgttctgggcaccgactacacctggccgtcgccatccagcattgacttcttcatgggcaagggaatgaataccttccgtattccgttcctcatggagcgtcttgtcccccctgccactggcatcacaggacctctcgaccagacgtacttgggcggcctgcagacgattgtcaactacatcaccggcaaaggcggctttgctctcattgacccgcacaactttatgatctacaatggccagacgatctccagtaccagcgacttccagaagttctggcagaacctcgcaggagtgtttaaatcgaacagtcacgtcatcttcgatgttatgaacgagcctcacgatattcccgcccagaccgtgttccaactgaaccaagccgctgtcaatggcatccgtgcgagcggtgcgacgtcgcagctcattctggtcgagggcacaagctggactggagcctggacctggacgacctctggcaacagcgatgcattcggtgccattaaggatcccaacaacaacgtcgcgatccagatgcatcagtacctggatagcgatggctctggcacttcgcagacctgcgtgtctcccaccatcggtgccgagcggttgcaggctgcgactcaatggttgaagcagaacaacctcaagggcttcctgggcgagatcggcgccggctctaactccgcttgcatcagcgctgtgcagggtgcgttgtgttcgatgcagcaatctggtgtgtggctcggcgctctctggtgggctgcgggcccgtggtggggcgactactaccagtccatcgagccgccctctggcccggcggtgtccgcgatcctcccgcaggccctgctgccgttcgcgtaa

SEQ ID NO: 16 - protein, 397 aa, BASIDIOMYCETE CBS 495.95
MKSLFLSLVATVALSSPVFSVAVWGQCGGIGFSGSTVCDAGAGCVKLNDYYSQCQPGAPTATSAAPSSNAPSGTSTASAPSSSLCSGSRTPFQFFGVNESGAEFGNLNIPGVLGTDYTWPSPSSIDFFMGKGMNTFRIPFLMERLVPPATGITGPLDQTYLGGLQTIVNYITGKGGFALIDPHNFMIYNGQTISSTSDFQKFWQNLAGVFKSNSHVIFDVMNEPHDIPAQTVFQLNQAAVNGIRASGATSQLILVEGTSWTGAWTWTTSGNSDAFGAIKDPNNNVAIQMHQYLDSDGSGTSQTCVSPTIGAERLQAATQWLKQNNLKGFLGEIGAGSNSACISAVQGALCSMQQSGVWLGALWWAAGPWWGDYYQSIEPPSGPAVSAILPQALLPFA

SEQ ID NO: 17 - DNA, 1303 bp, BASIDIOMYCETE CBS 495.95
ggaaagcgtcagtatggtgaaatttgcgcttgtggcaactgtcggcgcaatcttgagcgcttctgcggccaatgcggcttctatctaccagcaatgtggaggcattggatggtctgggtccactgtttgcgacgccggtctcgcttgcgttatcctcaatgcgtactactttcagtgcttgacgcccgccgcgggccagacaacgacgggctcgggcgcaccggcgtcaacatcaacctctcactcaacggtcactacggggagctcacactcaacaaccgggacgacggcgacgaaaacaactaccactccgtcgaccaccacgaccctacccgccatctctgtgtctggtcgcgtctgctctggctccaggacgaagttcaagttcttcggtgtgaatgaaagcggcgccgaattcgggaacactgcttggccagggcagctcgggaaagactatacatggccttcgcctagcagcgtggactacttcatgggggctggattcaatacattccgtatcaccttcttgatggagcgtatgagccctccggctaccggactcactggcccattcaaccagacgtacctgtcgggcctcaccaccattgtcgactacatcacgaacaaaggaggatacgctcttattgacccccacaacttcatgcgttacaacaacggcataatcagcagcacatctgacttcgcgacttggtggagcaatttggccactgtattcaaatccacgaagaacgccatcttcgacatccagaacgagccgtacggaatcgatgcgcagaccgtatacgaactgaatcaagctgccatcaattcgatccgcgccgctggcgctacgtcacagttgattctggttgaaggaacgtcatacactggagcttggacgtgggtctcgtccggaaacggagctgctttcgcggccgttacggatccttacaacaacacggcaattgaaatgcaccaatacctcgacagcgacggttctgggacaaacgaagactgtgtctcctccaccattgggtcgcaacgtctccaagctgccactgcgtggctgcaacaaacaggactcaagggattcctcggagagacgggtgctgggtcgaattcccagtgcatcgacgccgtgttcgatgaactttgctatatgcaacagcaaggcggctcctggatcggtgcactctggtgggctgcgggtccctggtggggcacgtacatttactcgattgaacctccgagcggtgccgctatcccagaagtccttcctcagggtctcgctccattcctctag

SEQ ID NO: 18 - protein, 429 aa, BASIDIOMYCETE CBS 495.95
MVKFALVATVGAILSASAANAASIYQQCGGIGWSGSTVCDAGLACVILNAYYFQCLTPAAGQTTTGSGAPASTSTSHSTVTTGSSHSTTGTTATKTTTTPSTTTTLPAISVSGRVCSGSRTKFKFFGVNESGAEFGNTAWPGQLGKDYTWPSPSSVDYFMGAGFNTFRITFLMERMSPPATGLTGPFNQTYLSGLTTIVDYITNKGGYALIDPHNFMRYNNGIISSTSDFATWWSNLATVFKSTKNAIFDIQNEPYGIDAQTVYELNQAAINSIRAAGATSQLILVEGTSYTGAWTWVSSGNGAAFAAVTDPYNNTAIEMHQYLDSDGSGTNEDCVSSTIGSQRLQAATAWLQQTGLKGFLGETGAGSNSQCIDAVFDELCYMQQQGGSWIGALWWAAGPWWGTYIYSIEPPSGAAIPEVLPQGLAPFL

SEQ ID NO: 19 - DNA, 1580 bp, Thielavia terrestris
agccccccgttcaggcacacttggcatcagatcagcttagcagcgcctgcacagcatgaagctctcgcagtcggccgcgctggcggcactcaccgcgacggcgctcgccgccccctcgcccacgacgccgcaggcgccgaggcaggcttcagccggctgctcgtctgcggtcacgctcgacgccagcaccaacgtttggaagaagtacacgctgcaccccaacagctactaccgcaaggaggttgaggccgcggtggcgcagatctcggacccggacctcgccgccaaggccaagaaggtggccgacgtcggcaccttcctgtggctcgactcgatcgagaacatcggcaagctggagccggcgatccaggacgtgccctgcgagaacatcctgggcctggtcatctacgacctgccgggccgcgactgcgcggccaaggcgtccaacggcgagctcaaggtcggcgagatcgaccgctacaagaccgagtacatcgacagtgagtgctgccccccgggttcgagaagagcgtgggggaaagggaaagggttgactgactgacacggcgcactgcagagatcgtgtcgatcctcaaggcacaccccaacacggcgttcgcgctggtcatcgagccggactcgctgcccaacctggtgaccaacagcaacttggacacgtgctcgagcagcgcgtcgggctaccgcgaaggcgtggcttacgccctcaagaacctcaacctgcccaacgtgatcatgtacctcgacgccggccacggcggctggctcggctgggacgccaacctgcagcccggcgcgcaggagctagccaaggcgtacaagaacgccggctcgcccaagcagctccgcggcttctcgaccaacgtggccggctggaactcctggtgagcttttttccattccatttcttcttcctcttctctcttcgctcccactctgcagccccccctcccccaagcacccactggcgttccggcttgctgactcggcctccctttccccgggcaccagggatcaatcgcccggcgaattctcccaggcgtccgacgccaagtacaacaagtgccagaacgagaagatctacgtcagcaccttcggctccgcgctccagtcggccggcatgcccaaccacgccatcgtcgacacgggccgcaacggcgtcaccggcctgcgcaaggagtggggtgactggtgcaacgtcaacggtgcaggttcgttgtcttctttttctcctcttttgtttgcacgtcgtggtccttttcaagcagccgtgtttggttgggggagatggactccggctgatgttctgcttcctctctaggcttcggcgtgcgcccgacgagcaacacgggcctcgagctggccgacgcgttcgtgtgggtcaagcccggcggcgagtcggacggcaccagcgacagctcgtcgccgcgctacgacagcttctgcggcaaggacgacgccttcaagccctcgcccgaggccggcacctggaacgaggcctacttcgagatgctgctcaagaacgccgtgccgtcgttctaagacggtccagcatcatccgg

SEQ ID NO: 20 - protein, 396 aa, Thielavia terrestris
MKLSQSAALAALTATALAAPSPTTPQAPRQASAGCSSAVTLDASTNVWKKYTLHPNSYYRKEVEAAVAQISDPDLAAKAKKVADVGTFLWLDSIENIGKLEPAIQDVPCENILGLVIYDLPGRDCAAKASNGELKVGEIDRYKTEYIDKIVSILKAHPNTAFALVIEPDSLPNLVTNSNLDTCSSSASGYREGVAYALKNLNLPNVIMYLDAGHGGWLGWDANLQPGAQELAKAYKNAGSPKQLRGFSTNVAGWNSWDQSPGEFSQASDAKYNKCQNEKIYVSTFGSALQSAGMPNHAIVDTGRNGVTGLRKEWGDWCNVNGAGFGVRPTSNTGLELADAFVWVKPGGESDGTSDSSSPRYDSFCGKDDAFKPSPEAGTWNEAYFEMLLKNAVPSF

SEQ ID NO: 21 - DNA, 1203 bp, Thielavia terrestris
atgaagtacctcaacctcctcgcagctctcctcgccgtcgctcctctctccctcgctgcacccagcatcgaggccagacagtcgaacgtcaacccatacatcggcaagagcccgctcgttattaggtcgtacgcccaaaagcttgaggagaccgtcaggaccttccagcaacgtggcgaccagctcaacgctgcgaggacacggacggtgcagaacgttgcgactttcgcctggatctcggataccaatggtattggagccattcgacctctcatccaagatgctctcgcccagcaggctcgcactggacagaaggtcatcgtccaaatcgtcgtctacaacctcccagatcgcgactgctctgccaacgcctcgactggagagttcaccgtaggaaacgacggtctcaaccgatacaagaactttgtcaacaccatcgcccgcgagctctcgactgctgacgctgacaagctccactttgccctcctcctcgaacccgacgcacttgccaacctcgtcaccaacgcgaatgcccccaggtgccgaatcgccgctcccgcttacaaggagggtatcgcctacaccctcgccaccttgtccaagcccaacgtcgacgtctacatcgacgccgccaacggtggctggctcggctggaacgacaacctccgccccttcgccgaactcttcaaggaagtctacgacctcgcccgccgcatcaaccccaacgccaaggtccgcggcgtccccgtcaacgtctccaactacaaccagtaccgcgctgaagtccgcgagcccttcaccgagtggaaggacgcctgggacgagagccgctacgtcaacgtcctcaccccgcacctcaacgccgtcggcttctccgcgcacttcatcgttgaccagggacgcggtggcaagggcggtatcaggacggagtggggccagtggtgcaacgttaggaacgctgggttcggtatcaggcctactgcggatcagggcgtgctccagaacccgaatgtggatgcgattgtgtgggttaagccgggtggagagtcggatggcacgagtgatttgaactcgaacaggtatgatcctacgtgcaggagtccggtggcgcatgttcccgctcctgaggctggccagtggttcaacgagtatgttgttaacctcgttttgaacgctaacccccctcttgagcctacctggtaa

SEQ ID NO: 22 - protein, 400 aa, Thielavia terrestris
MKYLNLLAALLAVAPLSLAAPSIEARQSNVNPYIGKSPLVIRSYAQKLEETVRTFQQRGDQLNAARTRTVQNVATFAWISDTNGIGAIRPLIQDALAQQARTGQKVIVQIVVYNLPDRDCSANASTGEFTVGNDGLNRYKNFVNTIARELSTADADKLHFALLLEPDALANLVTNANAPRCRIAAPAYKEGIAYTLATLSKPNVDVYIDAANGGWLGWNDNLRPFAELFKEVYDLARRINPNAKVRGVPVNVSNYNQYRAEVREPFTEWKDAWDESRYVNVLTPHLNAVGFSAHFIVDQGRGGKGGIRTEWGQWCNVRNAGFGIRPTADQGVLQNPNVDAIVWVKPGGESDGTSDLNSNRYDPTCRSPVAHVPAPEAGQWFNEYVVNLVLNANPPLEPTW

SEQ ID NO: 23 - DNA, 1501 bp, Thielavia terrestris
gccgttgtcaagatgggccagaagacgctgcacggattcgccgccacggctttggccgttctcccctttgtgaaggctcagcagcccggcaacttcacgccggaggtgcacccgcaactgccaacgtggaagtgcacgaccgccggcggctgcgttcagcaggacacttcggtggtgctcgactggaactaccgttggatccacaatgccgacggcaccgcctcgtgcacgacgtccagcggggtcgaccacacgctgtgtccagatgaggcgacctgcgcgaagaactgcttcgtggaaggcgtcaactacacgagcagcggtgtcaccacatccggcagttcgctgacgatgaggcagtatttcaaggggagcaacgggcagaccaacagcgtttcgcctcgtctctacctgctcggctcggatggaaactacgtaatgctcaagctgctcggccaggagctgagcttcgatgtcgatctctccacgctcccctgcggcgagaacggcgcgctgtacctgtccgagatggacgcgaccggtggcaggaaccagtacaacaccggcggtgccaactacggctcgggctactgtgacgcccagtgtcccgtgcagacgtggatgaacggcacgctgaacaccaacgggcagggctactgctgcaacgagatggacatcctcgaggccaactcccgcgccaacgcgatgacacctcacccctgcgccaacggcagctgcgacaagagcgggtgcggactcaacccctacgccgagggctacaagagctactacggaccgggcctcacggttgacacgtcgaagcccttcaccatcattacccgcttcatcaccgacgacggcacgaccagcggcaccctcaaccagatccagcggatctatgtgcagaatggcaagacggtcgcgtcggctgcgtccggaggcgacatcatcacggcatccggctgcacctcggcccaggcgttcggcgggctggccaacatgggcgcggcgcttggacggggcatggtgctgaccttcagcatctggaacgacgctgggggctacatgaactggctcgacagcggcaacaacggcccgtgcagcagcaccgagggcaacccgtccaacatcctggccaactacccggacacccacgtggtcttctccaacatccgctggggagacatcggctcgacggtccaggtctcgggaggcggcaacggcggctcgaccaccaccacgtcgaccaccacgctgaggacctcgaccacgaccaccaccaccgccccgacggccactgccacgcactggggacaatgcggcggaatcggggtacgtcaaccgcctcctgcattctgttgaggaagttaactaacgtggcctacgcagtggactggaccgaccgtctgcgaatcgccgtacgcatgcaaggagctgaacccctggtactaccagtgcctctaaagtattgcagtgaagccatactccgtgctcggcatgg

SEQ ID NO: 24 - protein, 464 aa, Thielavia terrestris
MGQKTLHGFAATALAVLPFVKAQQPGNFTPEVHPQLPTWKCTTAGGCVQQDTSVVLDWNYRWIHNADGTASCTTSSGVDHTLCPDEATCAKNCFVEGVNYTSSGVTTSGSSLTMRQYFKGSNGQTNSVSPRLYLLGSDGNYVMLKLLGQELSFDVDLSTLPCGENGALYLSEMDATGGRNQYNTGGANYGSGYCDAQCPVQTWMNGTLNTNGQGYCCNEMDILEANSRANAMTPHPCANGSCDKSGCGLNPYAEGYKSYYGPGLTVDTSKPFTIITRFITDDGTTSGTLNQIQRIYVQNGKTVASAASGGDIITASGCTSAQAFGGLANMGAALGRGMVLTFSIWNDAGGYMNWLDSGNNGPCSSTEGNPSNILANYPDTHVVFSNIRWGDIGSTVQVSGGGNGGSTTTTSTTTLRTSTTTTTTAPTATATHWGQCGGIGWTGPTVCESPYACKELNPWYYQCL

SEQ ID NO: 25 - DNA, 1368 bp, Thielavia terrestris
accgatccgctcgaagatggcgcccaagtctacagttctggccgcctggctgctctcctcgctggccgcggcccagcagatcggcaaagccgtgcccgaggtccaccccaaactgacaacgcagaagtgcactctccgcggcgggtgcaagcctgtccgcacctcggtcgtgctcgactcgtccgcgcgctcgctgcacaaggtcggggaccccaacaccagctgcagcgtcggcggcgacctgtgctcggacgcgaagtcgtgcggcaagaactgcgcgctcgagggcgtcgactacgcggcccacggcgtggcgaccaagggcgacgccctcacgctgcaccagtggctcaagggggccgacggcacctacaggaccgtctcgccgcgcgtatacctcctgggcgaggacgggaagaactacgaggacttcaagctgctcaacgccgagctcagcttcgacgtcgacgtgtcccagctcgtctgcggcatgaacggcgccctgtacttctccgagatggagatggacggcggccgcagcccgctgaacccggcgggcgccacgtacggcacgggctactgcgacgcgcagtgccccaagttggactttatcaacggcgaggtatttcttctctcttctgtttttcttttccatcgctttttctgaccggaatccgccctcttagctcaacaccaaccacacgtacggggcgtgctgcaacgagatggacatctgggaggccaacgcgctggcgcaggcgctcacgccgcacccgtgcaacgcgacgcgggtgtacaagtgcgacacggcggacgagtgcgggcagccggtgggcgtgtgcgacgaatgggggtgctcgtacaacccgtccaacttcggggtcaaggactactacgggcgcaacctgacggtggacacgaaccgcaagttcacggtgacgacgcagttcgtgacgtccaacgggcgggcggacggcgagctgaccgagatccggcggctgtacgtgcaggacggcgtggtgatccagaaccacgcggtcacggcgggcggggcgacgtacgacagcatcacggacggcttctgcaacgcgacggccacctggacgcagcagcggggcgggctcgcgcgcatgggcgaggccatcggccgcggcatggtgctcatcttcagcctgtgggttgacaacggcggcttcatgaactggctcgacagcggcaacgccgggccctgcaacgccaccgagggcgacccggccctgatcctgcagcagcacccggacgccagcgtcaccttctccaacatccgatggggcgagatcggcagcacgtacaagagcgagtgcagccactagagtagagcttgtaatt

SEQ ID NO: 26 - protein, 423 aa, Thielavia terrestris
MAPKSTVLAAWLLSSLAAAQQIGKAVPEVHPKLTTQKCTLRGGCKPVRTSVVLDSSARSLHKVGDPNTSCSVGGDLCSDAKSCGKNCALEGVDYAAHGVATKGDALTLHQWLKGADGTYRTVSPRVYLLGEDGKNYEDFKLLNAELSFDVDVSQLVCGMNGALYFSEMEMDGGRSPLNPAGATYGTGYCDAQCPKLDFINGELNTNHTYGACCNEMDIWEANALAQALTPHPCNATRVYKCDTADECGQPVGVCDEWGCSYNPSNFGVKDYYGRNLTVDTNRKFTVTTQFVTSNGRADGELTEIRRLYVQDGVVIQNHAVTAGGATYDSITDGFCNATATWTQQRGGLARMGEAIGRGMVLIFSLWVDNGGFMNWLDSGNAGPCNATEGDPALILQQHPDASVTFSNIRWGEIGSTYKSECSH

SEQ ID NO: 27 - DNA, 1011 bp, Thielavia terrestris
atgaccctacggctccctgtcatcagcctgctggcctcgctggcagcaggcgccgtcgtcgtcccacgggcggagtttcacccccctctcccgacttggaaatgcacgacctccgggggctgcgtgcagcagaacaccagcgtcgtcctggaccgtgactcgaagtacgccgcacacagcgccggctcgcggacggaatcggattacgcggcaatgggagtgtccacttcgggcaatgccgtgacgctgtaccactacgtcaagaccaacggcaccctcgtccccgcttcgccgcgcatctacctcctgggcgcggacggcaagtacgtgcttatggacctcctcaaccaggagctgtcggtggacgtcgacttctcggcgctgccgtgcggcgagaacggggccttctacctgtccgagatggcggcggacgggcggggcgacgcgggggcgggcgacgggtactgcgacgcgcagtgccagggctactgctgcaacgagatggacatcctcgaggccaactcgatggcgacggccatgacgccgcacccgtgcaagggcaacaactgcgaccgcagcggctgcggctacaacccgtacgccagcggccagcgcggcttctacgggcccggcaagacggtcgacacgagcaagcccttcaccgtcgtcacgcagttcgccgccagcggcggcaagctgacccagatcacccgcaagtacatccagaacggccgggagatcggcggcggcggcaccatctccagctgcggctccgagtcttcgacgggcggcctgaccggcatgggcgaggcgctggggcgcggaatggtgctggccatgagcatctggaacgacgcggcccaggagatggcatggctcgatgccggcaacaacggcccttgcgccagtggccagggcagcccgtccgtcattcagtcgcagcatcccgacacccacgtcgtcttctccaacatcaggtggggcgacatcgggtctaccacgaagaactag

SEQ ID NO: 28 - protein, 336 aa, Thielavia terrestris
MTLRLPVISLLASLAAGAVVVPRAEFHPPLPTWKCTTSGGCVQQNTSVVLDRDSKYAAHSAGSRTESDYAAMGVSTSGNAVTLYHYVKTNGTLVPASPRIYLLGADGKYVLMDLLNQELSVDVDFSALPCGENGAFYLSEMAADGRGDAGAGDGYCDAQCQGYCCNEMDILEANSMATAMTPHPCKGNNCDRSGCGYNPYASGQRGFYGPGKTVDTSKPFTVVTQFAASGGKLTQITRKYIQNGREIGGGGTISSCGSESSTGGLTGMGEALGRGMVLAMSIWNDAAQEMAWLDAGNNGPCASGQGSPSVIQSQHPDTHVVFSNIRWGDIGSTTKN

SEQ ID NO: 29 - DNA, 1480 bp, Cladorrhinum foecundissimum
gatccgaattcctcctctcgttctttagtcacagaccagacatctgcccacgatggttcacaagttcgccctcctcaccggcctcgccgcctccctcgcatctgcccagcagatcggcaccgtcgtccccgagtctcaccccaagcttcccaccaagcgctgcactctcgccggtggctgccagaccgtcgacacctccatcgtcatcgacgccttccagcgtcccctccacaagatcggcgacccttccactccttgcgtcgtcggcggccctctctgccccgacgccaagtcctgcgctgagaactgcgcgctcgagggtgtcgactatgcctcctggggcatcaagaccgagggcgacgccctaactctcaaccagtggatgcccgacccggcgaaccctggccagtacaagacgactactccccgtacttaccttgttgctgaggacggcaagaactacgaggatgtgaagctcctggctaaggagatctcgtttgatgccgatgtcagcaaccttccctgcggcatgaacggtgctttctacttgtctgagatgttgatggatggtggacgtggcgacctcaaccctgctggtgccgagtatggtaccggttactgtgatgcgcagtgcttcaagttggatttcatcaacggcgaggccaacatcgaccaaaagcacggcgcctgctgcaacgaaatggacattttcgaatccaactcgcgcgccaagaccttcgtcccccacccctgcaacatcacgcaggtctacaagtgcgaaggcgaagacgagtgcggccagcccgtcggcgtgtgcgacaagtgggggtgcggcttcaacgagtacaaatggggcgtcgagtccttctacggccggggctcgcagttcgccatcgactcctccaagaagttcaccgtcaccacgcagttcctgaccgacaacggcaaggaggacggcgtcctcgtcgagatccgccgcttgtggcaccaggatggcaagctgatcaagaacaccgctatccaggttgaggagaactacagcacggactcggtgagcaccgagttctgcgagaagactgcttctttcaccatgcagcgcggtggtctcaaggcgatgggcgaggctatcggtcgtggtatggtgctggttttcagcatctgggcggatgattcgggttttatgaactggttggatgcggagggtaatggcccttgcagcgcgactgagggcgatccgaaggagattgtcaagaataagccggatgctagggttacgttctcaaacattaggattggtgaggttggtagcacgtatgctccgggtgggaagtgcggtgttaagagcagggttgctagggggcttactgcttcttaaggggggtgtgaagagaggaggaggtgttgttgggggttggagatgataattgggcgagatggtgtagagcgggttggttggatatgaatacgttgaattggatgt

SEQ ID NO: 30 - protein, 440 aa, Cladorrhinum foecundissimum
MVHKFALLTGLAASLASAQQIGTVVPESHPKLPTKRCTLAGGCQTVDTSIVIDAFQRPLHKIGDPSTPCVVGGPLCPDAKSCAENCALEGVDYASWGIKTEGDALTLNQWMPDPANPGQYKTTTPRTYLVAEDGKNYEDVKLLAKEISFDADVSNLPCGMNGAFYLSEMLMDGGRGDLNPAGAEYGTGYCDAQCFKLDFINGEANIDQKHGACCNEMDIFESNSRAKTFVPHPCNITQVYKCEGEDECGQPVGVCDKWGCGFNEYKWGVESFYGRGSQFAIDSSKKFTVTTQFLTDNGKEDGVLVEIRRLWHQDGKLIKNTAIQVEENYSTDSVSTEFCEKTASFTMQRGGLKAMGEAIGRGMVLVFSIWADDSGFMNWLDAEGNGPCSATEGDPKEIVKNKPDARVTFSNIRIGEVGSTYAPGGKCGVKSRVARGLTAS

SEQ ID NO: 31 - DNA, 1380 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctttag

SEQ ID NO: 32 - protein, 459 aa, Trichoderma reesei; identical to SEQ ID NO: 4

SEQ ID NO: 33 - DNA, 1545 bp, Trichoderma reesei
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 34 - protein, 514 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 35 - DNA, 1611 bp, Trichoderma reesei
atgattgtcggcattctcaccacgctggctacgctggccacactcgcagctagtgtgcctctagaggagcggcaagcttgctcaagcgtctggtaattatgtgaaccctctcaagagacccaaatactgagatatgtcaaggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctgtaggtcctcccggaaccaaggcaatctgttactgaaggctcatcattcactgcagagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattggtatgagtttaaacacctgcctccccccccccttcccttcctttcccgccggcatcttgtcgttgtgctaactattgttccctcttccagagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacgtcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtaa

SEQ ID NO: 36 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGRLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 37 - DNA, 2046 bp, Humicola insolens
gccgtgaccttgcgcgctttgggtggcggtggcgagtcgtggacggtgcttgctggtcgccggccttcccggcgatccgcgtgatgagagggccaccaacggcgggatgatgctccatggggaacttccccatggagaagagagagaaacttgcggagccgtgatctggggaaagatgctccgtgtctcgtctatataactcgagtctccccgagccctcaacaccaccagctctgatctcaccatccccatcgacaatcacgcaaacacagcagttgtcgggccattccttcagacacatcagtcaccctccttcaaaatgcgtaccgccaagttcgccaccctcgccgcccttgtggcctcggccgccgcccagcaggcgtgcagtctcaccaccgagaggcacccttccctctcttggaacaagtgcaccgccggcggccagtgccagaccgtccaggcttccatcactctcgactccaactggcgctggactcaccaggtgtctggctccaccaactgctacacgggcaacaagtgggatactagcatctgcactgatgccaagtcgtgcgctcagaactgctgcgtcgatggtgccgactacaccagcacctatggcatcaccaccaacggtgattccctgagcctcaagttcgtcaccaagggccagcactcgaccaacgtcggctcgcgtacctacctgatggacggcgaggacaagtatcagagtacgttctatcttcagccttctcgcgccttgaatcctggctaacgtttacacttcacagccttcgagctcctcggcaacgagttcaccttcgatgtcgatgtctccaacatcggctgcggtctcaacggcgccctgtacttcgtctccatggacgccgatggtggtctcagccgctatcctggcaacaaggctggtgccaagtacggtaccggctactgcgatgctcagtgcccccgtgacatcaagttcatcaacggcgaggccaacattgagggctggaccggctccaccaacgaccccaacgccggcgcgggccgctatggtacctgctgctctgagatggatatctgggaagccaacaacatggctactgccttcactcctcacccttgcaccatcattggccagagccgctgcgagggcgactcgtgcggtggcacctacagcaacgagcgctacgccggcgtctgcgaccccgatggctgcgacttcaactcgtaccgccagggcaacaagaccttctacggcaagggcatgaccgtcgacaccaccaagaagatcactgtcgtcacccagttcctcaaggatgccaacggcgatctcggcgagatcaagcgcttctacgtccaggatggcaagatcatccccaactccgagtccaccatccccggcgtcgagggcaattccatcacccaggactggtgcgaccgccagaaggttgcctttggcgacattgacgacttcaaccgcaagggcggcatgaagcagatgggcaaggccctcgccggccccatggtcctggtcatgtccatctgggatgaccacgcctccaacatgctctggctcgactcgaccttccctgtcgatgccgctggcaagcccggcgccgagcgcggtgcctgcccgaccacctcgggtgtccctgctgaggttgaggccgaggcccccaacagcaacgtcgtcttctccaacatccgcttcggccccatcggctcgaccgttgctggtctccccggcgcgggcaacggcggcaacaacggcggcaaccccccgccccccaccaccaccacctcctcggctccggccaccaccaccaccgccagcgctggccccaaggctggccgctggcagcagtgcggcggcatcggcttcactggcccgacccagtgcgaggagccctacatttgcaccaagctcaacgactggtactctcagtgcctgtaaattctgagtcgctgactcgacgatcacggccggtttttgcatgaaaggaaacaaacgaccgcgataaaaatggagggtaatgagatgtc

SEQ ID NO: 38 - protein, 525 aa, Humicola insolens
MRTAKFATLAALVASAAAQQACSLTTERHPSLSWNKCTAGGQCQTVQASITLDSNWRWTHQVSGSTNCYTGNKWDTSICTDAKSCAQNCCVDGADYTSTYGITTNGDSLSLKFVTKGQHSTNVGSRTYLMDGEDKYQTFELLGNEFTFDVDVSNIGCGLNGALYFVSMDADGGLSRYPGNKAGAKYGTGYCDAQCPRDIKFINGEANIEGWTGSTNDPNAGAGRYGTCCSEMDIWEANNMATAFTPHPCTIIGQSRCEGDSCGGTYSNERYAGVCDPDGCDFNSYRQGNKTFYGKGMTVDTTKKITVVTQFLKDANGDLGEIKRFYVQDGKIIPNSESTIPGVEGNSITQDWCDRQKVAFGDIDDFNRKGGMKQMGKALAGPMVLVMSIWDDHASNMLWLDSTFPVDAAGKPGAERGACPTTSGVPAEVEAEAPNSNVVFSNIRFGPIGSTVAGLPGAGNGGNNGGNPPPPTTTTSSAPATTTTASAGPKAGRWQQCGGIGFTGPTQCEEPYICTKLNDWYSQCL

SEQ ID NO: 39 - DNA, 1812 bp, Myceliophthora thermophila
atggccaagaagcttttcatcaccgccgcccttgcggctgccgtgttggcggcccccgtcattgaggagcgccagaactgcggcgctgtgtggtaagaaagcccggtctgagtttcccatgactttctcatcgagtaatggcataaggcccaccccttcgactgactgtgagaatcgatcaaatccaggactcaatgcggcggcaacgggtggcagggtcccacatgctgcgcctcgggctcgacctgcgttgcgcagaacgagtggtactctcagtgcctgcccaacaatcaggtgacgagttccaacactccgtcgtcgacttccacctcgcagcgcagcagcagcacctccagcagcagcaccaggagcggcagctcctcctcctccaccaccacgccccctcccgtctccagccccgtgactagcattcccggcggtgcgaccaccacggcgagctactctggcaaccccttctcgggcgtccggctcttcgccaacgactactacaggtccgaggtccacaatctcgccattcctagcatgaccggtactctggcggccaaggcttccgccgtcgccgaagtccctagcttccagtggctcgaccggaacgtcaccatcgacaccctgatggtccagactctgtcccagatccgggctgccaataatgccggtgccaatcctccctatgctggtgagttacatggcggcgacttgccttctcgtcccccacctttcttgacgggatcggttacctgacctggaggcaaaacaaaaccagcccaacttgtcgtctacgacctccccgaccgtgactgcgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgacctgtttgccggcatctacaatgacgccggcaagccggctgccgtccgcggcctggccactaacgtcgccaactacaacgcctggagtatcgcttcggccccgtcgtacacgtcccctaaccctaactacgacgagaagcactacatcgaggccttcagcccgctcctgaacgcggccggcttccccgcacgcttcattgtcgacactggccgcaacggcaaacaacctaccggtatggtttttttctttttttttctctgttcccctcccccttccccttcagttggcgtccacaaggtctcttagtcttgcttcttctcggaccaaccttcccccacccccaaaacgcaccgcccacaaccgttcgactctatactcttgggaatgggcgccgaaactgaccgttcgacaggccaacaacagtggggtgactggtgcaatgtcaagggcactggctttggcgtgcgcccgacggccaacacgggccacgacctggtcgatgcctttgtctgggtcaagcccggcggcgagtccgacggcacaagcgacaccagcgccgcccgctacgactaccactgcggcctgtccgatgccctgcagcctgctccggaggctggacagtggttccaggcctacttcgagcagctgctcaccaacgccaacccgcccttctaa

SEQ ID NO: 40 - protein, 482 aa, Myceliophthora thermophila
MAKKLFITAALAAAVLAAPVIEERQNCGAVWTQCGGNGWQGPTCCASGSTCVAQNEWYSQCLPNNQVTSSNTPSSTSTSQRSSSTSSSSTRSGSSSSSTTTPPPVSSPVTSIPGGATTTASYSGNPFSGVRLFANDYYRSEVHNLAIPSMTGTLAAKASAVAEVPSFQWLDRNVTIDTLMVQTLSQIRAANNAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAADLFAGIYNDAGKPAAVRGLATNVANYNAWSIASAPSYTSPNPNYDEKHYIEAFSPLLNAAGFPARFIVDTGRNGKQPTGQQQWGDWCNVKGTGFGVRPTANTGHDLVDAFVWVKPGGESDGTSDTSAARYDYHCGLSDALQPAPEAGQWFQAYFEQLLTNANPPF

SEQ ID NO: 41 - DNA, 1802 bp, Myceliophthora thermophila
atggccaagaagcttttcatcaccgccgcgcttgcggctgccgtgttggcggcccccgtcattgaggagcgccagaactgcggcgctgtgtggtaagaaagcccggtccgagtctcccatgattttctcgtcgagtaatggcataagggccaccccttcgactgaccgtgagaatcgatcaaatccaggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccccgcccgtctccagccccgtgaccagcattcccggcggtgcgacctccacggcgagctactctggcaaccccttctcgggcgtccggctcttcgccaacgactactacaggtccgaggtccacaatctcgccattcctagcatgactggtactctggcggccaaggcttccgccgtcgccgaagtccctagcttccagtggctcgaccggaacgtcaccatcgacaccctgatggtccagactctgtcccaggtccgggctctcaataaggccggtgccaatcctccctatgctggtgagttacatggcgacttgccttctcgtcccctacctttcttgacgggatcggttacctgacctggaggcaaaacaacaacagcccaactcgtcgtctacgacctccccgaccgtgactgtgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgtcgcctaaccctaactacgacgagaagcactacatcgaggccttcagcccgctcttgaactcggccggcttccccgcacgcttcattgtcgacactggccgcaacggcaaacaacctaccggtatgtttttttttcttttgtctctgtcccccccttttctcccccttcagttggcgtccacaaggtctcttagtcctgcttcatctgtgaccaacctccccccccccggcaccgcccacaaccgtttgactctatactcttgggaatgggcgccgaaactgaccgttccacaggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttctaa

SEQ ID NO: 42 - protein, 481 aa, Myceliophthora thermophila
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPP

SEQ ID NO: 43 - DNA, 1446 bp, Thielavia terrestris
atggctcagaagctccttctcgccgccgcccttgcggccagcgccctcgctgctcccgtcgtcgaggagcgccagaactgcggttccgtctggagccaatgcggcggcattggctggtccggcgcgacctgctgcgcttcgggcaatacctgcgttgagctgaacccgtactactcgcagtgcctgcccaacagccaggtgactacctcgaccagcaagaccacctccaccaccaccaggagcagcaccaccagccacagcagcggtcccaccagcacgagcaccaccaccaccagcagtcccgtggtcactaccccgccgagtacctccatccccggcggtgcctcgtcaacggccagctggtccggcaacccgttctcgggcgtgcagatgtgggccaacgactactacgcctccgaggtctcgtcgctggccatccccagcatgacgggcgccatggccaccaaggcggccgaggtggccaaggtgcccagcttccagtggcttgaccgcaacgtcaccatcgacacgctgttcgcccacacgctgtcgcagatccgcgcggccaaccagaaaggcgccaacccgccctacgcgggcatcttcgtggtctacgaccttccggaccgcgactgcgccgccgccgcgtccaacggcgagttctccatcgcgaacaacggggcggccaactacaagacgtacatcgacgcgatccggagcctcgtcatccagtactcagacatccgcatcatcttcgtcatcgagcccgactcgctggccaacatggtgaccaacctgaacgtggccaagtgcgccaacgccgagtcgacctacaaggagttgaccgtctacgcgctgcagcagctgaacctgcccaacgtggccatgtacctggacgccggccacgccggctggctcggctggcccgccaacatccagccggccgccaacctcttcgccgagatctacacgagcgccggcaagccggccgccgtgcgcggcctcgccaccaacgtggccaactacaacggctggagcctggccacgccgccctcgtacacccagggcgaccccaactacgacgagagccactacgtccaggccctcgccccgctgctcaccgccaacggcttccccgcccacttcatcaccgacaccggccgcaacggcaagcagccgaccggacaacggcaatggggagactggtgcaacgttatcggaactggcttcggcgtgcgcccgacgacaaacaccggcctcgacatcgaggacgccttcgtctgggtcaagcccggcggcgagtgcgacggcacgagcaacacgacctctccccgctacgactaccactgcggcctgtcggacgcgctgcagcctgctccggaggccggcacttggttccaggcctacttcgagcagctcctgaccaacgccaacccgcccttttaa

SEQ ID NO: 44 - protein, 481 aa, Thielavia terrestris
MAQKLLLAAALAASALAAPVVEERQNCGSVWSQCGGIGWSGATCCASGNTCVELNPYYSQCLPNSQVTTSTSKTTSTTTRSSTTSHSSGPTSTSTTTTSSPVVTTPPSTSIPGGASSTASWSGNPFSGVQMWANDYYASEVSSLAIPSMTGAMATKAAEVAKVPSFQWLDRNVTIDTLFAHTLSQIRAANQKGANPPYAGIFVVYDLPDRDCAAAASNGEFSIANNGAANYKTYIDAIRSLVIQYSDIRIIFVIEPDSLANMVTNLNVAKCANAESTYKELTVYALQQLNLPNVAMYLDAGHAGWLGWPANIQPAANLFAEIYTSAGKPAAVRGLATNVANYNGWSLATPPSYTQGDPNYDESHYVQALAPLLTANGFPAHFITDTGRNGKQPTGQRQWGDWCNVIGTGFGVRPTTNTGLDIEDAFVWVKPGGECDGTSNTTSPRYDYHCGLSDALQPAPEAGTWFQAYFEQLLTNANPPF

SEQ ID NO: 45 - DNA, 1593 bp, Chaetomium thermophilum
atgatgtacaagaagttcgccgctctcgccgccctcgtggctggcgccgccgcccagcaggcttgctccctcaccactgagacccaccccagactcacttggaagcgctgcacctctggcggcaactgctcgaccgtgaacggcgccgtcaccatcgatgccaactggcgctggactcacactgtttccggctcgaccaactgctacaccggcaacgagtgggatacctccatctgctctgatggcaagagctgcgcccagacctgctgcgtcgacggcgctgactactcttcgacctatggtatcaccaccagcggtgactccctgaacctcaagttcgtcaccaagcaccagcacggcaccaatgtcggctctcgtgtctacctgatggagaacgacaccaagtaccagatgttcgagctcctcggcaacgagttcaccttcgatgtcgatgtctctaacctgggctgcggtctcaacggcgccctctacttcgtctccatggacgctgatggtggtatgagcaagtactctggcaacaaggctggcgccaagtacggtaccggctactgcgatgctcagtgcccgcgcgaccttaagttcatcaacggcgaggccaacattgagaactggaccccttcgaccaatgatgccaacgccggtttcggccgctatggcagctgctgctctgagatggatatctgggatgccaacaacatggctactgccttcactcctcacccttgcaccattatcggccagagccgctgcgagggcaacagctgcggtggcacctacagctctgagcgctatgctggtgtttgcgatcctgatggctgcgacttcaacgcctaccgccagggcgacaagaccttctacggcaagggcatgaccgtcgacaccaccaagaagatgaccgtcgtcacccagttccacaagaactcggctggcgtcctcagcgagatcaagcgcttctacgttcaggacggcaagatcattgccaacgccgagtccaagatccccggcaaccccggcaactccatcacccaggagtggtgcgatgcccagaaggtcgccttcggtgacatcgatgacttcaaccgcaagggcggtatggctcagatgagcaaggccctcgagggccctatggtcctggtcatgtccgtctgggatgaccactacgccaacatgctctggctcgactcgacctaccccattgacaaggccggcacccccggcgccgagcgcggtgcttgcccgaccacctccggtgtccctgccgagattgaggcccaggtccccaacagcaacgttatcttctccaacatccgcttcggccccatcggctcgaccgtccctggcctcgacggcagcacccccagcaacccgaccgccaccgttgctcctcccacttctaccaccaccagcgtgagaagcagcactactcagatttccaccccgactagccagcccggcggctgcaccacccagaagtggggccagtgcggtggtatcggctacaccggctgcactaactgcgttgctggcactacctgcactgagctcaacccctggtacagccagtgcctgtaa

SEQ ID NO: 46 - protein, 530 aa, Chaetomium thermophilum
MMYKKFAALAALVAGAAAQQACSLTTETHPRLTWKRCTSGGNCSTVNGAVTIDANWRWTHTVSGSTNCYTGNEWDTSICSDGKSCAQTCCVDGADYSSTYGITTSGDSLNLKFVTKHQHGTNVGSRVYLMENDTKYQMFELLGNEFTFDVDVSNLGCGLNGALYFVSMDADGGMSKYSGNKAGAKYGTGYCDAQCPRDLKFINGEANIENWTPSTNDANAGFGRYGSCCSEMDIWDANNMATAFTPHPCTIIGQSRCEGNSCGGTYSSERYAGVCDPDGCDFNAYRQGDKTFYGKGMTVDTTKKMTVVTQFHKNSAGVLSEIKRFYVQDGKIIANAESKIPGNPGNSITQEWCDAQKVAFGDIDDFNRKGGMAQMSKALEGPMVLVMSVWDDHYANMLWLDSTYPIDKAGTPGAERGACPTTSGVPAEIEAQVPNSNVIFSNIRFGPIGSTVPGLDGSTPSNPTATVAPPTSTTTSVRSSTTQISTPTSQPGGCTTQKWGQCGGIGYTGCTNCVAGTTCTELNPWYSQCL

SEQ ID NO: 47 - DNA, 1434 bp, Chaetomium thermophilum
atggctaagcagctgctgctcactgccgctcttgcggccacttcgctggctgcccctctccttgaggagcgccagagctgctcctccgtctggggtcaatgcggtggcatcaattacaacggcccgacctgctgccagtccggcagtgtttgcacttacctgaatgactggtacagccagtgcattcccggtcaggctcagcccggcacgactagcaccacggctcggaccaccagcaccagcaccaccagcacttcgtcggtccgcccgaccacctcgaatacccctgtgacgactgctcccccgacgaccaccatcccgggcggcgcctcgagcacggccagctacaacggcaacccgttttcgggtgttcaactttgggccaacacctactactcgtccgaggtgcacactttggccatccccagcttgtctcctgagctggctgccaaggccgccaaggtcgctgaggttcccagcttccagtggctcgaccgcaatgtgactgttgacactctcttctccggcactcttgccgaaatccgcgccgccaaccagcgcggtgccaacccgccttatgccggcattttcgtggtttatgacttaccagaccgtgattgcgcggctgctgcttcgaacggcgagtggtctatcgccaacaatggtgccaacaactacaagcgctacatcgaccggatccgtgagctccttatccagtactccgatatccgcactattctggtcattgaacctgattccctggccaacatggtcaccaacatgaacgtccagaagtgctcgaacgctgcctccacttacaaggagcttactgtctatgccctcaaacagctcaatcttcctcacgttgccatgtacatggatgctggccacgctggctggcttggctggcccgccaacatccagcctgctgctgagctctttgctcaaatctaccgcgacgctggcaggcccgctgctgtccgcggtcttgcgaccaacgttgccaactacaatgcttggtcgatcgccagccctccgtcctacacctctcctaacccgaactacgacgagaagcactatattgaggcctttgctcctcttctccgcaaccagggcttcgacgcaaagttcatcgtcgacaccggccgtaacggcaagcagcccactggccagcttgaatggggtcactggtgcaatgtcaagggaactggcttcggtgtgcgccctactgctaacactgggcatgaacttgttgatgctttcgtgtgggtcaagcccggtggcgagtccgacggcaccagtgcggacaccagcgctgctcgttatgactatcactgcggcctttccgacgcactgactccggcgcctgaggctggccaatggttccaggcttatttcgaacagctgctcatcaatgccaaccctccgctctga

SEQ ID NO: 48 - protein, 477 aa, Chaetomium thermophilum
MAKQLLLTAALAATSLAAPLLEERQSCSSVWGQCGGINYNGPTCCQSGSVCTYLNDWYSQCIPGQAQPGTTSTTARTTSTSTTSTSSVRPTTSNTPVTTAPPTTTIPGGASSTASYNGNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNYKRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSADTSAARYDYHCGLSDALTPAPEAGQWFQAYFEQLLINANPPL

SEQ ID NO: 49 - DNA, 1599 bp, Aspergillus fumigatus
atgctggcctccaccttctcctaccgcatgtacaagaccgcgctcatcctggccgcccttctgggctctggccaggctcagcaggtcggtacttcccaggcggaagtgcatccgtccatgacctggcagagctgcacggctggcggcagctgcaccaccaacaacggcaaggtggtcatcgacgcgaactggcgttgggtgcacaaagtcggcgactacaccaactgctacaccggcaacacctgggacacgactatctgccctgacgatgcgacctgcgcatccaactgcgcccttgagggtgccaactacgaatccacctatggtgtgaccgccagcggcaattccctccgcctcaacttcgtcaccaccagccagcagaagaacattggctcgcgtctgtacatgatgaaggacgactcgacctacgagatgtttaagctgctgaaccaggagttcaccttcgatgtcgatgtctccaacctcccctgcggtctcaacggtgctctgtactttgtcgccatggacgccgacggtggcatgtccaagtacccaaccaacaaggccggtgccaagtacggtactggatactgtgactcgcagtgccctcgcgacctcaagttcatcaacggtcaggccaacgtcgaagggtggcagccctcctccaacgatgccaatgcgggtaccggcaaccacgggtcctgctgcgcggagatggatatctgggaggccaacagcatctccacggccttcaccccccatccgtgcgacacgcccggccaggtgatgtgcaccggtgatgcctgcggtggcacctacagctccgaccgctacggcggcacctgcgaccccgacggatgtgatttcaactccttccgccagggcaacaagaccttctacggccctggcatgaccgtcgacaccaagagcaagtttaccgtcgtcacccagttcatcaccgacgacggcacctccagcggcaccctcaaggagatcaagcgcttctacgtgcagaacggcaaggtgatccccaactcggagtcgacctggaccggcgtcagcggcaactccatcaccaccgagtactgcaccgcccagaagagcctgttccaggaccagaacgtcttcgaaaagcacggcggcctcgagggcatgggtgctgccctcgcccagggtatggttctcgtcatgtccctgtgggatgatcactcggccaacatgctctggctcgacagcaactacccgaccactgcctcttccaccactcccggcgtcgcccgtggtacctgcgacatctcctccggcgtccctgcggatgtcgaggcgaaccaccccgacgcctacgtcgtctactccaacatcaaggtcggccccatcggctcgaccttcaacagcggtggctcgaaccccggtggcggaaccaccacgacaactaccacccagcctactaccaccacgaccacggctggaaaccctggcggcaccggagtcgcacagcactatggccagtgtggtggaatcggatggaccggacccacaacctgtgccagcccttatacctgccagaagctgaatgattattactctcagtgcctgtag

SEQ ID NO: 50 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 51 - DNA, 1713 bp, Aspergillus fumigatus
atgaagcaccttgcatcttccatcgcattgactctactgttgcctgccgtgcaggcccagcagaccgtatggggccaatgtatgttctggctgtcactggaataagactgtatcaactgctgatatgcttctaggtggcggccaaggctggtctggcccgacgagctgtgttgccggcgcagcctgtagcacactgaatccctgtatgttagatatcgtcctgagtggagacttatactgacttccttagactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctgtaagtggccttatcccaatactgagaccaactctctgacagtcgtagcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcggtaggccgtacacctccgttgcgcgccgcctttctctgacatcttgcagaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggtatgcctcacttcccgcattctgtatcccttccagacactaactcatcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctgtaagtgcttattccaatcgccgatgtgtgccgactaatcaatgtttcagcccggaatggcgtccagcccacgaagcaaaacgcctggggtgactggtgcaacgtcatcggcaccggcttcggtgttcgcccctcgactaacaccggcgatccgctccaggatgcctttgtgtggatcaagcccggtggagagagtgatggcacgtccaactcgacttccccccggtatgacgcgcactgcggatatagtgatgctctgcagcctgctcctgaggctggtacttggttccaggtatgtcatccattagccagatgagggataagtgactgacggacctaggcctactttgagcagcttctgaccaacgctaacccgtccttttaa

SEQ ID NO: 52 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 53 - DNA, 2586 bp, Aspergillus oryzae
atgaagcttggttggatcgaggtggccgcattggcggctgcctcagtagtcagtgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 54 - protein, 861 aa, Aspergillus oryzae
MKLGWIEVAALAAASVVSAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 55 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 56 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 57 - DNA, 2800 bp, Penicillium brasilianum
tgaaaatgcagggttctacaatctttctggctttcgcctcatgggcgagccaggttgctgccattgcgcagcccatacagaagcacgaggtttgttttatcttgctcatggacgtgctttgacttgactaattgttttacatacagcccggatttctgcacgggccccaagccatagaatcgttctcagaaccgttctacccgtcgccctggatgaatcctcacgccgagggctgggaggccgcatatcagaaagctcaagattttgtctcgcaactcactatcttggagaaaataaatctgaccaccggtgttgggtaagtctctccgactgcttctgggtcacggtgcgacgagccactgactttttgaagctgggaaaatgggccgtgtgtaggaaacactggatcaattcctcgtctcggattcaaaggattttgtacccaggattcaccacagggtgttcggttcgcagattattcctccgctttcacatctagccaaatggccgccgcaacatttgaccgctcaattctttatcaacgaggccaagccatggcacaggaacacaaggctaagggtatcacaattcaattgggccctgttgccggccctctcggtcgcatccccgagggcggccgcaactgggaaggattctcccctgatcctgtcttgactggtatagccatggctgagacaattaagggcatgcaggatactggagtgattgcttgcgctaaacattatattggaaacgagcaggagcacttccgtcaagtgggtgaagctgcgggtcacggatacactatttccgatactatttcatctaatattgacgaccgtgctatgcatgagctatacttgtggccatttgctgatgccgttcgcgctggtgtgggttctttcatgtgctcatactctcagatcaacaactcctacggatgccaaaacagtcagaccctcaacaagctcctcaagagcgaattgggcttccaaggctttgtcatgagcgattggggtgcccatcactctggagtgtcatcggcgctagctggacttgatatgagcatgccgggtgataccgaatttgattctggcttgagcttctggggctctaacctcaccattgcaattctgaacggcacggttcccgaatggcgcctggatgacatggcgatgcgaattatggctgcatacttcaaagttggccttactattgaggatcaaccagatgtcaacttcaatgcctggacccatgacacctacggatataaatacgcttatagcaaggaagattacgagcaggtcaactggcatgtcgatgttcgcagcgaccacaataagctcattcgcgagactgccgcgaagggtacagttctgctgaagaacaactttcatgctctccctctgaagcagcccaggttcgtggccgtcgttggtcaggatgccgggccaaaccccaagggccctaacggctgcgcagaccgaggatgcgaccaaggcactctcgcaatgggatggggctcagggtctaccgaattcccttacctggtcactcctgacactgctattcagtcaaaggtcctcgaatacgggggtcgatacgagagtatttttgataactatgacgacaatgctatcttgtcgcttgtctcacagcctgatgcaacctgtatcgtttttgcaaatgccgattccggtgaaggctacatcactgtcgacaacaactggggtgaccgcaacaatctgaccctctggcaaaatgccgatcaagtgattagcactgtcagctcgcgatgcaacaacacaatcgttgttctccactctgtcggaccagtgttgctaaatggtatatatgagcacccgaacatcacagctattgtctgggcagggatgccaggcgaagaatctggcaatgctctcgtggatattctttggggcaatgttaaccctgccggtcgcactccgttcacctgggccaaaagtcgagaggactatggcactgatataatgtacgagcccaacaacggccagcgtgcgcctcagcaggatttcaccgagagcatctacctcgactaccgccatttcgacaaagctggtatcgagccaatttacgagtttggattcggcctctcctataccaccttcgaatactctgacctccgtgttgtgaagaagtatgttcaaccatacagtcccacgaccggcaccggtgctcaagcaccttccatcggacagccacctagccagaacctggatacctacaagttccctgctacatacaagtacatcaaaaccttcatttatccctacctgaacagcactgtctccctccgcgctgcttccaaggatcccgaatacggtcgtacagactttatcccaccccacgcgcgtgatggctcccctcaacctctcaaccccgctggagacccagtggccagtggtggaaacaacatgctctacgacgaactttacgaggtcactgcacagatcaaaaacactggcgacgtggccggcgacgaagtcgtccagctttacgtagatctcgggggtgacaacccgcctcgtcagttgagaaactttgacaggttttatctgctgcccggtcagagctcaacattccgggctacattgacgcgccgtgatttgagcaactgggatattgaggcgcagaactggcgagttacggaatcgcctaagagagtgtatgttggacggtcgagtcgggatttgccgctgagctcacaattggagtaatgatcatgtctaccaatagatgttgaatgtctggtgtggatatt

SEQ ID NO: 58 - protein, 878 aa, Penicillium brasilianum
MQGSTIFLAFASWASQVAAIAQPIQKHEPGFLHGPQAIESFSEPFYPSPWMNPHAEGWEAAYQKAQDFVSQLTILEKINLTTGVGWENGPCVGNTGSIPRLGFKGFCTQDSPQGVRFADYSSAFTSSQMAAATFDRSILYQRGQAMAQEHKAKGITIQLGPVAGPLGRIPEGGRNWEGFSPDPVLTGIAMAETIKGMQDTGVIACAKHYIGNEQEHFRQVGEAAGHGYTISDTISSNIDDRAMHELYLWPFADAVRAGVGSFMCSYSQINNSYGCQNSQTLNKLLKSELGFQGFVMSDWGAHHSGVSSALAGLDMSMPGDTEFDSGLSFWGSNLTIAILNGTVPEWRLDDMAMRIMAAYFKVGLTIEDQPDVNFNAWTHDTYGYKYAYSKEDYEQVNWHVDVRSDHNKLIRETAAKGTVLLKNNFHALPLKQPRFVAVVGQDAGPNPKGPNGCADRGCDQGTLAMGWGSGSTEFPYLVTPDTAIQSKVLEYGGRYESIFDNYDDNAILSLVSQPDATCIVFANADSGEGYITVDNNWGDRNNLTLWQNADQVISTVSSRCNNTIVVLHSVGPVLLNGIYEHPNITAIVWAGMPGEESGNALVDILWGNVNPAGRTPFTWAKSREDYGTDIMYEPNNGQRAPQQDFTESIYLDYRHFDKAGIEPIYEFGFGLSYTTFEYSDLRVVKKYVQPYSPTTGTGAQAPSIGQPPSQNLDTYKFPATYKYIKTFIYPYLNSTVSLRAASKDPEYGRTDFIPPHARDGSPQPLNPAGDPVASGGNNMLYDELYEVTAQIKNTGDVAGDEVVQLYVDLGGDNPPRQLRNFDRFYLLPGQSSTFRATLTRRDLSNWDIEAQNWRVTESPKRVYVGRSSRDLPLSSQLE

SEQ ID NO: 59 - DNA, 2583 bp, Aspergillus niger
atgaggttcactttgatcgaggcggtggctctgactgccgtctcgctggccagcgctgatgaattggcctactccccaccgtattacccatccccttgggccaatggccagggcgactgggcgcaggcataccagcgcgctgttgatattgtctcgcaaatgacattggatgagaaggtcaatctgaccacaggaactggatgggaattggaactatgtgttggtcagactggcggtgttccccgattgggagttccgggaatgtgtttacaggatagccctctgggcgttcgcgactccgactacaactctgctttccctgccggcatgaacgtggctgcaacctgggacaagaatctggcataccttcgcggcaaggctatgggtcaggaatttagtgacaagggtgccgatatccaattgggtccagctgccggccctctcggtagaagtcccgacggtggtcgtaactgggagggcttctccccagaccctgccctaagtggtgtgctctttgccgagaccatcaagggtatccaagatgctggtgtggttgcgacggctaagcactacattgcttacgagcaagagcatttccgtcaggcgcctgaagcccaaggttttggatttaatatttccgagagtggaagtgcgaacctcgatgataagactatgcacgagctgtacctctggcccttcgcggatgccatccgtgcaggtgctggcgctgtgatgtgctcctacaaccagatcaacaacagttatggctgccagaacagctacactctgaacaagctgctcaaggccgagctgggcttccagggctttgtcatgagtgattgggctgctcaccatgctggtgtgagtggtgctttggcaggattggatatgtctatgccaggagacgtcgactacgacagtggtacgtcttactggggtacaaacttgaccattagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactacgtgtcggagggaccgtacgagaaggtcaaccagtacgtgaatgtgcaacgcaaccacagcgaactgattcgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgcttaagcacaagaatggtgtattcaccgccaccgataactgggctatcgatcagattgaggcgcttgctaagaccgccagtgtctctcttgtctttgtcaacgccgactctggtgagggttacatcaatgtggacggaaacctgggtgaccgcaggaacctgaccctgtggaggaacggcgataatgtgatcaaggctgctgctagcaactgcaacaacacaatcgttgtcattcactctgtcggaccagtcttggttaacgagtggtacgacaaccccaatgttaccgctatcctctggggtggtttgcccggtcaggagtctggcaactctcttgccgacgtcctctatggccgtgtcaaccccggtgccaagtcgccctttacctggggcaagactcgtgaggcctaccaagactacttggtcaccgagcccaacaacggcaacggagcccctcaggaagactttgtcgagggcgtcttcattgactaccgtggatttgacaagcgcaacgagaccccgatctacgagttcggctatggtctgagctacaccactttcaactactcgaaccttgaggtgcaggtgctgagcgcccctgcatacgagcctgcttcgggtgagaccgaggcagcgccaaccttcggagaggttggaaatgcgtcggattacctctaccccagcggattgcagagaattaccaagttcatctacccctggctcaacggtaccgatctcgaggcatcttccggggatgctagctacgggcaggactcctccgactatcttcccgagggagccaccgatggctctgcgcaaccgatcctgcctgccggtggcggtcctggcggcaaccctcgcctgtacgacgagctcatccgcgtgtcagtgaccatcaagaacaccggcaaggttgctggtgatgaagttccccaactgtatgtttcccttggcggtcccaatgagcccaagatcgtgctgcgtcaattcgagcgcatcacgctgcagccgtcggaggagacgaagtggagcacgactctgacgcgccgtgaccttgcaaactggaatgttgagaagcaggactgggagattacgtcgtatcccaagatggtgtttgtcggaagctcctcgcggaagctgccgctccgggcgtctctgcctactgttcactaa

SEQ ID NO: 60 - protein, 860 aa, Aspergillus niger
MRFTLIEAVALTAVSLASADELAYSPPYYPSPWANGQGDWAQAYQRAVDIVSQMTLDEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGFGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKHKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASDYLYPSGLQRITKFIYPWLNGTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 61 - DNA, 2583 bp, Aspergillus aculeatus
atgaagctcagttggcttgaggcggctgccttgacggctgcttcagtcgtcagcgctgatgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggaattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaagcttctgaaggccgagctcggcttccagggctttgtgatgtctgactggggtgctcaccacagtggtgttggctctgctttggccggcttggatatgtcaatgcctggcgatatcaccttcgattctgccactagtttctggggtaccaacctgaccattgctgtgctcaacggtaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggatccaactcgtacggtgccaatggctgctctgaccgtggctgtgacaacggtactcttgctatggcttggggtagcggcactgccgaattcccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccgggcgccaaatctccattcacctggggcaagacgagggaggcgtacggggattaccttgtccgtgagctcaacaacggcaacggagctccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaatga

SEQ ID NO: 62 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 63 - DNA, 3294 bp, Aspergillus oryzae
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggtggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatccccatgcgttcctcccccctcctccgctccgccgttgtggccgccctgccggtgttggcccttgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 64 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 65 - DNA, 3294 bp, Aspergillus oryzae
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggtggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatccccatgcgttcctcccccctcctccgctccgccgttgtggccgccctgccggtgttggcccttgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaagtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgttatgtgctcttacaaccaaatcaacaacagctacggttgcgagaatagcgaaactctgaacaagcttttgaaggcggagcttggtttccaaggcttcgtcatgagtgattggaccgctcaacacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 66 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWESFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAQHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 67 - DNA, 1846 bp, Thielavia terrestris
aattgaaggagggagtggcggagtggccaccaagtcaggcggctgtcaactaaccaaggatgggaacagttcggctcgccttgcccgagggcagcgttccctgatggggacgaaccatgggactggggtcagctgctgtataaaagttcaaatcgatgatctctcagatggcgctgctggggtgttctgcgcttttccatcctcgcaacctggtatcccactagtccagcgttcggcaccatgaagtcgttcaccattgccgccttggcagccctatgggcccaggaggccgccgcccacgcgaccttccaggacctctggattgatggagtcgactacggctcgcaatgtgtccgcctcccggcgtccaactcccccgtcaccaatgttgcgtccgacgatatccgatgcaatgtcggcacctcgaggcccaccgtcaagtgcccggtcaaggccggctccacggtcacgatcgagatgcaccaggttcgcacgcctctctgcgtaggccccccagctactatatggcactaacacgacctccagcaacctggcgaccggtcttgcgccaacgaggctatcggcggcgaccactacggccccgtaatggtgtacatgtccaaggtcgatgacgcggtgacagccgacggttcatcgggctggttcaaggtgttccaggacagctgggccaagaacccgtcgggttcgacgggcgacgacgactactggggcaccaaggacctcaactcgtgctgcggcaagatgaacgtcaagatccccgaagacatcgagccgggcgactacctgctccgcgccgaggttatcgcgctgcacgtggccgccagctcgggcggcgcgcagttctacatgtcctgctaccagctgaccgtgacgggctccggcagcgccaccccctcgaccgtgaatttcccgggcgcctactcggccagcgacccgggcatcctgatcaacatccacgcgcccatgtcgacctacgtcgtcccgggcccgaccgtgtacgcgggcggctcgaccaagtcggctggcagctcctgctccggctgcgaggcgacctgcacggttggttccggccccagcgcgacactgacgcagcccacctccaccgcgaccgcgacctccgcccctggcggcggcggctccggctgcacggcggccaagtaccagcagtgcggcggcaccggctacactgggtgcaccacctgcgctgtaagttccctcgtgatatgcagcggaacaccgtctggactgttttgctaactcgcgtcgtagtccgggtctacctgcagcgccgtctcgcctccgtactactcgcagtgcctctaagccgggagcgcttgctcagcgggctgctgtgaaggagctccatgtccccatgccgccatggccggagtaccgggctgagcgcccaattcttgtatatagttgagttttcccaatcatgaatacatatgcatctgcatggactgttgcgtcgtcagtctacatcctttgctccactgaactgtgagaccccatgtcatccggaccattcgatcggtgctcgctctaccatctcggttgatgggtctgggcttgagagtcactggcacgtcctcggcggtaatgaaatgtggaggaaagtgtgagctgtctgacgcactcggcgctgatgagacgttgagcgcggcccacactggtgttctgtaagccagcacacaaaagaatactccaggatggcccatagcggcaaatatacagtatcagggatgcaaaaagtgcaaaagtaaggggctcaatcggggatcgaacccgagacctcgcacatgacttatttcaagtcaggggt

SEQ ID NO: 68 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 69 - DNA, 880 bp, Thielavia terrestris
accccgggatcactgcccctaggaaccagcacacctcggtccaatcatgcggttcgacgccctctccgccctcgctcttgcgccgcttgtggctggccacggcgccgtgaccagctacatcatcggcggcaaaacctatcccggctacgagggcttctcgcctgcctcgagcccgccgacgatccagtaccagtggcccgactacaacccgaccctgagcgtgaccgacccgaagatgcgctgcaacggcggcacctcggcagagctcagcgcgcccgtccaggccggcgagaacgtgacggccgtctggaagcagtggacccaccagcaaggccccgtcatggtctggatgttcaagtgccccggcgacttctcgtcgtgccacggcgacggcaagggctggttcaagatcgaccagctgggcctgtggggcaacaacctcaactcgaacaactggggcaccgcgatcgtctacaagaccctccagtggagcaacccgatccccaagaacctcgcgccgggcaactacctcatccgccacgagctgctcgccctgcaccaggccaacacgccgcagttctacgccgagtgcgcccagctggtcgtctccggcagcggctccgccctgcccccgtccgactacctctacagcatccccgtctacgcgccccagaacgaccccggcatcaccgtgagtgggcttccgttccgcggcgagctctgtggaaatcttgctgacgatgggctaggttgacatctacaacggcgggcttacctcctacaccccgcccggcggccccgtctggtctggcttcgagttttaggcgcattgagtcgggggctacgaggggaaggcatctgttcgcatgagcgtgggtac

SEQ ID NO: 70 - protein, 239 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 71 - DNA, 1000 bp, Thielavia terrestris
ctcctgttcctgggccaccgcttgttgcctgcactattggtagagttggtctattgctagagttggccatgcttctcacatcagtcctcggctcggctgccctgcttgctagcggcgctgcggcacacggcgccgtgaccagctacatcatcgccggcaagaattacccggggtgggtagctgattattgagggcgcattcaaggttcataccggtgtgcatggctgacaaccggctggcagataccaaggcttttctcctgcgaactcgccgaacgtcatccaatggcaatggcatgactacaaccccgtcttgtcgtgcagcgactcgaagcttcgctgcaacggcggcacgtcggccaccctgaacgccacggccgcaccgggcgacaccatcaccgccatctgggcgcagtggacgcacagccagggccccatcctggtgtggatgtacaagtgcccgggctccttcagctcctgtgacggctccggcgctggctggttcaagatcgacgaggccggcttccacggcgacggcgtcaaggtcttcctcgacaccgagaacccgtccggctgggacatcgccaagctcgtcggcggcaacaagcagtggagcagcaaggtccccgagggcctcgcccccggcaactacctcgtccgccacgagttgatcgccctgcaccaggccaacaacccgcagttctacccggagtgcgcccaggtcgtcatcaccggctccggcaccgcgcagccggatgcctcatacaaggcggctatccccggctactgcaaccagaatgacccgaacatcaaggtgagatccaggcgtaatgcagtctactgctggaaagaaagtggtccaagctaaaccgcgctccaggtgcccatcaacgaccactccatccctcagacctacaagattcccggccctcccgtcttcaagggcaccgccagcaagaaggcccgggacttcaccgcctgaagttgttgaatcgatggag

SEQ ID NO: 72 - protein, 258 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 73 - DNA, 681 bp, Thielavia terrestris
atgctcgcaaacggtgccatcgtcttcctggccgccgccctcggcgtcagtggccactacacctggccacgggttaacgacggcgccgactggcaacaggtccgtaaggcggacaactggcaggacaacggctacgtcggggatgtcacgtcgccacagatccgctgtttccaggcgaccccgtccccggccccatccgtcctcaacaccacggccggctcgaccgtgacctactgggccaaccccgacgtctaccaccccgggcctgtgcagttttacatggcccgcgtgcccgatggcgaggacatcaactcgtggaacggcgacggcgccgtgtggttcaaggtgtacgaggaccatcctacctttggcgctcagctcacatggcccagcacgggcaagagctcgttcgcggttcccatccccccgtgcatcaagtccggctactacctcctccgggcggagcaaatcggcctgcacgtcgcccagagcgtaggcggagcgcagttctacatctcatgcgcccagctcagcgtcaccggcggcggcagcaccgagccgccgaacaaggtggccttccccggcgcttacagtgcgacggacccgggcattctgatcaacatctactaccctgttcccacgtcctaccagaaccccggcccggccgtcttcagctgctga

SEQ ID NO: 74 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 75 - DNA, 960 bp, Thielavia terrestris
atgaagggacttttcagtgccgccgccctctccctggccgtcggccaggcttcggcccattacatcttccagcaactctccatcaacgggaaccagtttccggtgtaccaatatattcgcaagaacaccaattataacagtcccgttaccgatctcacgtccgacgatcttcggtgcaatgtcggcgcccagggtgctgggacagacaccgtcacggtgaaggccggcgaccagttcaccttcacccttgacacccctgtttaccaccaggggcccatctccatctacatgtccaaggccccgggcgcggcgtcagactacgatggcagcggcggctggttcaagatcaaggactggggcccgactttcaacgccgacggcacggccacctgggacatggccggctcatacacctacaacatcccgacctgcattcccgacggcgactatctgctccgcatccagtcgctggccatccacaacccctggccggcgggcatcccgcagttctacatctcctgcgcccagatcaccgtgaccggcggcggcaacggcaaccctggcccgacggccctcatccccggcgccttcaaggacaccgacccgggctacacggtgaacatctacacgaacttccacaactacacggttcccggcccggaggtcttcagctgcaacggcggcggctcgaacccgcccccgccggtgagtagcagcacgcccgcgaccacgacgctggtcacgtcgacgcgcaccacgtcctccacgtcctccgcctcgacgccggcctcgaccggcggctgcaccgtcgccaagtggggccagtgcggcggcaacgggtacaccggctgcacgacctgcgcggccgggtccacctgcagcaagcagaacgactactactcgcagtgcttgtaagggaggccgcaaagcatgaggtgtttgaagaggaggagaggggtc

SEQ ID NO: 76 - protein, 304 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 77 - DNA, 954 bp, Thielavia terrestris
atgaagggcctcagcctcctcgccgctgcgtcggcagcgactgctcataccatcttcgtgcagctcgagtcagggggaacgacctatccggtatcctacggcatccgggaccctagctacgacggtcccatcaccgacgtcacctccgactcactggcttgcaatggtcccccgaaccccacgacgccgtccccgtacatcatcaacgtcaccgccggcaccacggtcgcggcgatctggaggcacaccctcacatccggccccgacgatgtcatggacgccagccacaaggggccgaccctggcctacctcaagaaggtcgatgatgccttgaccgacacgggtatcggcggcggctggttcaagatccaggaggccggttacgacaatggcaattgggctaccagcacggtgatcaccaacggtggcttccaatatattgacatccccgcctgcattcccaacggccagtatctgctccgcgccgagatgatcgcgctccacgccgccagcacgcagggtggtgcccagctctacatggagtgcgcgcagatcaacgtggtgggcggctccggcagcgccagcccgcagacgtacagcatcccgggcatctaccaggcaaccgacccgggcctgctgatcaacatctactccatgacgccgtccagccagtacaccattccgggtccgcccctgttcacctgcagcggcagcggcaacaacggcggcggcagcaacccgtcgggcgggcagaccacgacggcgaagcccacgacgacgacggcggcgacgaccacctcctccgccgctcctaccagcagccaggggggcagcagcggttgcaccgttccccagtggcagcagtgcggtggcatctcgttcaccggctgcaccacctgcgcggcgggctacacctgcaagtatctgaacgactattactcgcaatgccagtaa

SEQ ID NO: 78 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 79 - DNA, 799 bp, Thermoascus aurantiacus
atgtccttttccaagataattgctactgccggcgttcttgcctctgcttctctagtggctggccatggcttcgttcagaacatcgtgattgatggtaaaaagtatgtcattgcaagacgcacataagcggcaacagctgacaatcgacagttatggcgggtatctagtgaaccagtatccatacatgtccaatcctccagaggtcatcgcctggtctactacggcaactgatcttggatttgtggacggtactggataccaaaccccagatatcatctgccataggggcgccaagcctggagccctgactgctccagtctctccaggaggaactgttgagcttcaatggactccatggcctgattctcaccatggcccagttatcaactaccttgctccgtgcaatggtgattgttccactgtggataagacccaattagaattcttcaaaattgccgagagcggtctcatcaatgatgacaatcctcctgggatctgggcttcagacaatctgatagcagccaacaacagctggactgtcaccattccaaccacaattgcacctggaaactatgttctgaggcatgagattattgctcttcactcagctcagaaccaggatggtgcccagaactatccccagtgcatcaatctgcaggtcactggaggtggttctgataaccctgctggaactcttggaacggcactctaccacgataccgatcctggaattctgatcaacatctatcagaaactttccagctatatcatccctggtcctcctctgtatactggttaa

SEQ ID NO: 80 - protein, 249 aa, Thermoascus aurantiacus
MSFSKIIATAGVLASASLVAGHGFVQNIVIDGKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 81 - DNA, 1172 bp, Trichoderma reesei
ggatctaagccccatcgatatgaagtcctgcgccattcttgcagcccttggctgtcttgccgggagcgttctcggccatggacaagtccaaaacttcacgatcaatggacaatacaatcagggtttcattctcgattactactatcagaagcagaatactggtcacttccccaacgttgctggctggtacgccgaggacctagacctgggcttcatctcccctgaccaatacaccacgcccgacattgtctgtcacaagaacgcggccccaggtgccatttctgccactgcagcggccggcagcaacatcgtcttccaatggggccctggcgtctggcctcacccctacggtcccatcgttacctacgtggctgagtgcagcggatcgtgcacgaccgtgaacaagaacaacctgcgctgggtcaagattcaggaggccggcatcaactataacacccaagtctgggcgcagcaggatctgatcaaccagggcaacaagtggactgtgaagatcccgtcgagcctcaggcccggaaactatgtcttccgccatgaacttcttgctgcccatggtgcctctagtgcgaacggcatgcagaactatcctcagtgcgtgaacatcgccgtcacaggctcgggcacgaaagcgctccctgccggaactcctgcaactcagctctacaagcccactgaccctggcatcttgttcaacccttacacaacaatcacgagctacaccatccctggcccagccctgtggcaaggctagatccaggggtacggtgttggcgttcgtgaagtcggagctgttgacaaggatatctgatgatgaacggagaggactgatgggcgtgactgagtgtatatatttttgatgaccaaattgtatacgaaatccgaacgcatggtgatcattgtttatccctgtagtatattgtctccaggctgctaagagcccaccgggtgtattacggcaacaaagtcaggaatttgggtggcaatgaacgcaggtctccatgaatgtatatgtgaagaggcatcggctggcatgggcattaccagatataggccctgtgaaacatatagtacttgaacgtgctactggaacggatcataagcaagtcatcaacatgtgaaaaaacactacatgtaaaaaaaaaaaaaaaaaaaaa

SEQ ID NO: 82 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 83 - DNA, 924 bp, Myceliophthora thermophila
atgaagttcacctcgtccctcgctgtcctggccgctgccggcgcccaggctcactgttagtcgaccctcgaacccaacacccccctccccccttttctcctccatctcctcggcctcacttagtagccgctgacaacgactagataccttccctagggccggcactggtggctcgctctctggcgagtgggaggtggtccgcatgaccgagaaccattactcgcacggcccggtcaccgatgtcaccagccccgagatgacctgctatcagtccggcgtgcagggtgcgccccagaccgtccaggtcaaggcgggctcccaattcaccttcagcgtggatccctcgatcggccaccccggccctctccagttctacatggctaaggtgccgtcgggccagacggccgccacctttgacggcacgggagccgtgtggttcaagatctaccaagacggcccgaacggcctcggcaccgacagcattacctggcccagcgccggttcgtgacttcctccccactcgcttttttttttttattttttatttttttttctttcggaactcaagaatctttctctctctctcccgtctttggccttgaacaacactaaaactcttccttactgtattaattaggcaaaaccgaggtctcggtcaccatccccagctgcatcgatgatggcgagtacctgctccgggtcgagcacatcgcgctccacagcgccagcagcgtgggcggcgctcagttctacattgcctgcgcccagctctccgtcaccggcggctccggcaccctcaacacgggctcgctcgtctccctgcccggcgcctacaaggccaccgacccgggcatcctcttccagctctactggcccatcccgaccgagtacatcaaccccggcccggcccccgtctcttgctaa

SEQ ID NO: 84 - protein, 232 aa, Myceliophthora thermophila
MKFTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIDDGEYLLRVEHIALHSASSVGGAQFYIACAQLSVTGGSGTLNTGSLVSLPGAYKATDPGILFQLYWPIPTEYINPGPAPVSC

SEQ ID NO: 85 - DNA, 854 bp, Myceliophthora thermophila
atgaaggccctctctctccttgcggctgcctcggcagtctctgcgcataccatcttcgtccagctcgaagcagacggcacgaggtacccggtctcgtacgggatccgggacccaagctacgacggccccatcaccgacgtcacatccaacgacgttgcttgcaacggcgggccgaacccgacgaccccctccagcgacgtcatcaccgtcaccgcgggcaccacggtcaaggccatctggaggcacaccctccaatccggcccggacgatgtcatggacgccagccacaagggcccgaccctggcctacctcaagaaggtcggcgatgccaccaaggactcgggcgtcggcggtggctggttcaagattcaggaggacggctacaacaacggccagtggggcaccagcaccgttatctccaacggcggcgagcactacatgtgagccattcctccgagagaagaccaagactcttgacgatctcgctgacccgtgcaacaagtgacatcccggcctgcatccccgagggtcagtacctcctccgcgccgagatgatcgccctccacgcggccgggtcccccggcggtgcccagctctacgtaagcctctgcccttccccccttcctcttgatcgaatcggactgcccaccccccttttcgactccgactaacaccgttgccagatggaatgtgcccagatcaacatcgtcggcggctccggctcggtgcccagctcgaccgtcagcttccccggcgcgtacagccccaacgacccgggtctcctcatcaacatctattccatgtcgccctcgagctcgtacaccatcccgggcccgcccgtcttcaagtgctag

SEQ ID NO: 86 - protein, 235 aa, Myceliophthora thermophila
MKALSLLAAASAVSAHTIFVQLEADGTRYPVSYGIRDPSYDGPITDVTSNDVACNGGPNPTTPSSDVITVTAGTTVKAIWRHTLQSGPDDVMDASHKGPTLAYLKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 87 - DNA, 1242 bp, Myceliophthora thermophila
atgaagtccttcgccctcaccactctggccgccctggccggcaacgccgccgctcacgcgaccttccaggccctctgggtcgacggcgtcgactacggcgcgcagtgtgcccgtctgcccgcgtccaactccccggtcaccgacgtgacctccaacgcgatccgctgcaacgccaacccgtcgcccgctcggggcaagtgcccggtcaaggccggctcgaccgttacggtcgagatgcatcaggtacgttggatgaatgaaaggggaaaggaagcagaggcagaaggggaaggcgaagggaaagaaaaagaaaaagaaatggaaaagaaaaagaaatggaaaagaaaaagaaaaatgaaaaagaaagtggaaaccgtcagactaactggggctcctcccccccacccctcctttgatatcagcaacccggtgaccggtcgtgcagcagcgaggcgatcggcggggcgcactacggccccgtcatggtgtacatgtccaaggtgtcggacgcggcgtcggcggacgggtcgtcgggctggttcaaggtgttcgaggacggctgggccaagaacccgtccggcgggtcgggcgacgacgactactggggcaccaaggacctgaactcgtgctgcgggaagatgaacgtcaagatccccgccgacctgccctcgggcgactacctgctccgggccgaggccctcgcgctgcacacggcgggcagcgccggcggcgcccagttctacatgacgtgctaccagctcaccgtgacgggctccggcagcgccagcccgcccaccgtctccttcccgggcgcctacaaggccaccgacccgggcatcctcgtcaacatccacgccccgctgtccggctacaccgtgcccggcccggccgtctactccggcggctccaccaagaaggccggcagcgcctgcaccggctgcgagtccacctgcgccgtcggctccggccccaccgccaccgtctcccagtcgcccggttccaccgccacctccgcccccggcggcggcggcggctgcaccgtccagaagtaccagcagtgcggcggcgagggctacaccggctgcaccaactgcgcggtacgtttttcaaccccgtttttttttttccttccctaccttatttggttacctaattaattactttccggctgctgactttttgctttagtccggctctacctgcagcgccgtctcgccgccctactactcgcagtgcgtctaa

SEQ ID NO: 88 - protein, 323 aa, Myceliophthora thermophila
MKSFALTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGEGYTGCTNCASGSTCSAVSPPYYSQCV

SEQ ID NO: 89 - DNA, 1253 bp, Myceliophthora thermophila
atgaagccttttagcctcgtcgccctggcgaccgccgtgagcggccatgccatcttccagcgggtgtcggtcaacgggcaggaccagggccagctcaagggggtgcgggcgccgtcgagcaactccccgatccagaacgtcaacgatgccaacatggcctgcaacgccaacattgtgtaccacgacagcaccatcatcaaggtgcccgcgggagcccgcgtcggcgcgtggtggcagcacgtcatcggcgggccgcagggcgccaacgacccggacaacccgatcgcggcctcccacaagggtatgatgatcgatgatgcctctctcttcccccgttcttgatggacaggcgatggctcccaggaacacgcgtgactgaccaccgaatccaggccccatccaggtctacctggccaaggtggacaacgcggcgacggcgtcgccgtcgggcctcaggtggttcaaggtggccgagcgcggcctgaacaacggcgtgtgggccgtcgatgagctcatcgccaacaacggctggcactacttcgacctgccgtcgtgcgtggcccccggccagtacctgatgcgcgtcgagctgctcgccctgcacagcgcctcaagccccggcggcgcccagttctacatgggctgcgcacagatcgaaggtgcgtcgatctttgttctccttccgtgtcctctctgatcctttctctcttctttttctttcttttactccctttccttccatcttcggagaagcaacgaagggggaaagggatagaagagaggaatgagagacgacgaaagagaggattggggaaagacaagacagggaaaaaaagacaagaaaaaaaaaaaaaaaaaaaaacagagtgagctaacaagaacaatcagtcactggctccggcaccaactcgggctccgactttgtctcgttccccggcgcctactcggccaacgatccgggcatcttgctaagcatctacgacagctcgggcaagcccaccaacggcgggcgctcgtacccgatccccggcccgcgccccatctcctgctccggcagcggcgacggcggcaacaacggcggcggcggcgacgacaacaacaataacaacggtggtggcaacaacggcggcggcggcggcggcagcgtccccctgtacgggcagtgcggcggcatcggctacacgggcccgaccacctgtgcccagggaacttgcaaggtgtcgaacgaatactacagccagtgcctcccctag

SEQ ID NO: 90 - protein, 310 aa, Myceliophthora thermophila
MKPFSLVALATAVSGHAIFQRVSVNGQDQGQLKGVRAPSSNSPIQNVNDANMACNANIVYHDSTIIKVPAGARVGAWWQHVIGGPQGANDPDNPIAASHKGPIQVYLAKVDNAATASPSGLRWFKVAERGLNNGVWAVDELIANNGWHYFDLPSCVAPGQYLMRVELLALHSASSPGGAQFYMGCAQIEVTGSGTNSGSDFVSFPGAYSANDPGILLSIYDSSGKPTNGGRSYPIPGPRPISCSGSGDGGNNGGGGDDNNNNNGGGNNGGGGGGSVPLYGQCGGIGYTGPTTCAQGTCKVSNEYYSQCLP

SEQ ID NO: 91 - DNA, 814 bp, Myceliophthora thermophila
atgaagctctccctcttctccgtcctggccactgccctcaccgtcgaggggcatgccatcttccagaaggtctccgtcaacggagcggaccagggctccctcaccggcctccgcgctcccaacaacaacaaccccgtgcaggatgtcaacagccaggacatgatctgcggccagtcgggatcgacgtcgaacactatcatcgaggtcaaggccggcgataggatcggtgcctggtatcagcatgtcatcggcggtgcccagttccccaacgacccagacaacccgattgccaagtcgcacaagggccccgtcatggcctacctcgccaaggttgacaatgccgcaaccgccagcaagacgggcctgaagtggtatgtattcccgcggcccgagggacatcgggttgggcaagtcgagactgacggagctcgcttctccgtataggttcaagatttgggaggatacctttaatcccagcaccaagacctggggtgtcgacaacctcatcaataacaacggctgggtgtacttcaacctcccgcagtgcatcgccgacggcaactacctcctccgcgtcgaggtcctcgctctgcactcggcctactctcagggccaggctcagttctaccagtcctgcgcccagatcaacgtatccggcggcggctccttcacaccgccgtcgactgtcagcttcccgggtgcctacagcgccagcgaccccggtatcctgatcaacatctacggcgccaccggccagcccgacaacaacggccagccgtacactgcccctgggcccgcgcccatctcctgctga

SEQ ID NO: 92 - protein, 246 aa, Myceliophthora thermophila
MKLSLFSVLATALTVEGHAIFQKVSVNGADQGSLTGLRAPNNNNPVQDVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPPSTVSFPGAYSASDPGILINIYGATGQPDNNGQPYTAPGPAPISC

SEQ ID NO: 93 - DNA, 1115 bp, Thermoascus aurantiacus
atgtcgttctcgaagattgctgcgatcaccggggccattacctatgcgtctctggccgccgctcacggttatgttacaggaatcgtagccgatggcacctagtatgtaacgctcatgccaagatccgcattgctgtactaacaattagcagctacgggggctatatcgtgacccaatacccctacatgtcgacaccgccggatgtcatcgcctggtctaccaaagcaactgatcttggtttcgtggatcccagtagctatgcttcgtctgatattatctgccacaagggtgctgagcctggtgccctgagcgccaaggtggctgctggagggaccgtcgagctgcagtggacggattggcctgagagtcacaagggcccggtcattgactacctcgccgcctgtaacggggactgctcgactgtcgacaagaccaaactagagttcttcaagattgatgagagtggcctaattgacggcagcagcgccccaggcacatgggcctctgacaacttgattgccaataacaacagctggaccgtcaccatcccgagcacgattgctcccggcaactatgtcctgagacatgaaatcattgccctccactccgccggaaatacaaatggtgctcagaactacccccagtgtatcaaccttgaggtcacaggcagtggcaccgacacccctgccggcaccctcggaacggagctttataaggcaacggaccctggcattctggtcaacatctaccagaccctgaccagctacgatattcccggccctgctctgtacaccggtggtagctctggtagctctggttcctccaacaccgccaaggccaccacttcgacggcttctagctctatcgtgaccccgacgcctgttaacaacccaaccgttactcagactgccgttgttgatgtcacccagactgtttcccagaatgctgccgtcgccaccacgactccggcctccactgcagttgctacagctgtcccaacgggaaccacctttagctttgattcgatgacctcggatgaattcgtcagcctgatgcgtgcgaccgtgaattggctgctttctaacaagaagcatgcccgggatctttcttactaa

SEQ ID NO: 94 - protein, 354 aa, Thermoascus aurantiacus
MSFSKIAAITGAITYASLAAAHGYVTGIVADGTYYGGYIVTQYPYMSTPPDVIAWSTKATDLGFVDPSSYASSDIICHKGAEPGALSAKVAAGGTVELQWTDWPESHKGPVIDYLAACNGDCSTVDKTKLEFFKIDESGLIDGSSAPGTWASDNLIANNNSWTVTIPSTIAPGNYVLRHEIIALHSAGNTNGAQNYPQCINLEVTGSGTDTPAGTLGTELYKATDPGILVNIYQTLTSYDIPGPALYTGGSSGSSGSSNTAKATTSTASSSIVTPTPVNNPTVTQTAVVDVTQTVSQNAAVATTTPASTAVATAVPTGTTFSFDSMTSDEFVSLMRATVNWLLSNKKHARDLSY

SEQ ID NO: 95 - DNA, 862 bp, Aspergillus fumigatus
atgactttgtccaagatcacttccattgctggccttctggcctcagcgtctctcgtggctggccacggctttgtttctggcattgttgctgatgggaaatagtatgtgcttgaaccacacaaatgacagctgcaacagctaacttctattccagttacggagggtaccttgttaaccaatacccctacatgagcaaccctcccgacaccattgcctggtccaccaccgccaccgacctcggctttgtggacggcaccggctaccagtctccggatattatctgccacagagacgcaaagaatggcaagttgaccgcaaccgttgcagccggttcacagatcgaattccagtggacgacgtggccagagtctcaccatggaccggtacgacgccgaagagaagagaacatattgtgaccagataggctaacatagcatagttgattacttacctcgctccatgcaacggcgactgtgccaccgtggacaagaccaccctgaagtttgtcaagatcgccgctcaaggcttgatcgacggctccaacccacctggtgtttgggctgatgatgaaatgatcgccaacaacaacacggccacagtgaccattcctgcctcctatgcccccggaaactacgtccttcgccacgagatcatcgcccttcactctgcgggtaacctgaacggcgcgcagaactacccccagtgtttcaacatccaaatcaccggtggcggcagtgctcagggatctggcaccgctggcacgtccctgtacaagaatactgatcctggcatcaagtttgacatctactcggatctgagcggtggataccctattcctggtcctgcactgttcaacgcttaa

SEQ ID NO: 96 - protein, 250 aa, Aspergillus fumigatus [sequence omitted]

SEQ ID NO: 97 - DNA, 1021 bp, Penicillium pinophilum
atgccttctactaaagtcgctgccctttctgctgttctagctttggcctccacggttgctggccatggttttgtgcaaaacatcgttatcgacggtaaatcgtaagcagtgatgcatccattattaaactagacatgcttacaaaaaaatcagttactctggataccttgtgaatcagttcccctacgagtccaacccaccagctgttattgggtgggcaacaactgcaaccgacctgggattcgtcgctcccagtgagtacaccaatgcagacattatctgccacaagaacgccacacctggcgcgctttctgctccagttgctgcagggggcactgtcgagctccagtggactacatggcccgatagtcatcacggtcctgtcatcagctacctcgccaactgcaatggcaattgttctaccgtggataagactaagctagactttgtcaagattgaccaaggtggtttgatcgacgatactacccccccgggtacatgggcttccgacaaacttatcgctgccaacaacagctggactgtaactatcccctccaccatcgcgcctggaaactacgttttgcgccacgaaatcattgctcttcactccgctggaaacgcagacggtgcccaaaactaccctcaatgcatcaacttggagatcaccggcagcggaaccgccgctccctctggtaccgctggcgaaaagctctacacctctactgaccccggtatcttggtcaatatctaccaatccttgtcgacctacgttattcccggaccaactctgtggagcggtgctgccaatggcgctgttgccactggttctgctactgcggttgctacgactgccactgcttctgcgaccgctactcctaccacacttgttacctctgtcgctccagcttcatctacctttgccactgctgttgtgaccactgtcgctcctgcagtaactgatgtcgtgactgtcaccgatgtagttaccgtgaccaccgtcatcaccactactgtcctttga

SEQ ID NO: 98 - protein, 322 aa, Penicillium pinophilum
MPSTKVAALSAVLALASTVAGHGFVQNIVIDGKSYSGYLVNQFPYESNPPAVIGWATTATDLGFVAPSEYTNADIICHKNATPGALSAPVAAGGTVELQWTTWPDSHHGPVISYLANCNGNCSTVDKTKLDFVKIDQGGLIDDTTPPGTWASDKLIAANNSWTVTIPSTIAPGNYVLRHEIIALHSAGNADGAQNYPQCINLEITGSGTAAPSGTAGEKLYTSTDPGILVNIYQSLSTYVIPGPTLWSGAANGAVATGSATAVATTATASATATPTTLVTSVAPASSTFATAVVTTVAPAVTDVVTVTDVVTVTTVITTTVL

SEQ ID NO: 99 - DNA, 1486 bp, Thermoascus sp.
atgttgtcgttcgcttctgccaagtcagctgtgctgacgacccttctacttcttggatccgctcaggctcacactttgatgaccaccctgtttgtggatggcgtcaatcagggagatggtgtctgtattcgcatgaacaacaacggtagtactgccaacacctatatccagcctgtcacgagcaaggatattgcctgcggtaagtacagtaccggtccagatatcatactctatttcaatccgacaacagtcagagctggagagcaatgctaaacatccccaggcattcaaggcgaaattggcgccgctcgagtctgtccagccaaggcttcatccaccctcacgttccaattccgagagcagccatccaacccgaattccgctcctctcgatccctcgcacaaaggccccgctgcggtgtacctgaaaaaggtagactccgccatcgcgagcaacaacgccgctggagacggctggttcaagatctgggagtccgtctacgacgagtccacgggcaaatggggtacgaccaagatgatcgagaacaacgggcacatctctgtcaaggtccccgacgatatcgagggtgggtattatctcgcgcgtacggagcttctggcgctgcacgcggcgaacgaaggggatccgcagttctacgttggctgcgcgcagctgttcatcgattcagcggggacagcgaaaccgcctactgtctctattggagaggggacctacgatctgagcatgcctgccatgacgtacaatatctaccagactccgttggctctaccatacccgatgtatgggcctcctgtctacacacctggctctggctcgggttctggctctggttccgggtcagcttctgcaacgagatcttctgctattcctactgccaccgctgttacggactgttcttccgaagaggacagggaagactcagtcatggcaaccggtgttcccgttgcaagaagcacactcagaacctgggttgacagactgtcatggcatggtaaggcccgtgagaacgtgaaaccagccgccaggagaagcgcccttgtccagaccgagggtctgaagccggaaggctgcatcttcgtcaacggcaactggtgcggtttcgaggtccccgattacaacgatgcggaaagctgctgggctgtacgttcccgtctaattacttaaaacgaaataaaagctaacagtacttttctttttctaatcccaggcctccgacaactgctggaaacagtccgactcgtgctggaaccagacccagcccaccggctacaacaactgccagatctggcaagaccagaaatgcaagcccatccaggactcgtgtagccaatccaacccgactggaccgccgaacaagggcaaggatataactccaacgtggccgcccctggagggctcgatgaagaccttcaccaagcgcactgtcagttaccgtgattggattatgaaaaggaaaggagcataa

SEQ ID NO: 100 - protein, 444 aa, Thermoascus sp.
MLSFASAKSAVLTTLLLLGSAQAHTLMTTLFVDGVNQGDGVCIRMNNNGSTANTYIQPVTSKDIACGIQGEIGAARVCPAKASSTLTFQFREQPSNPNSAPLDPSHKGPAAVYLKKVDSAIASNNAAGDGWFKIWESVYDESTGKWGTTKMIENNGHISVKVPDDIEGGYYLARTELLALHAANEGDPQFYVGCAQLFIDSAGTAKPPTVSIGEGTYDLSMPAMTYNIYQTPLALPYPMYGPPVYTPGSGSGSGSGSGSASATRSSAIPTATAVTDCSSEEDREDSVMATGVPVARSTLRTWVDRLSWHGKARENVKPAARRSALVQTEGLKPEGCIFVNGNWCGFEVPDYNDAESCWAASDNCWKQSDSCWNQTQPTGYNNCQIWQDQKCKPIQDSCSQSNPTGPPNKGKDITPTWPPLEGSMKTFTKRTVSYRDWIMKRKGA

SEQ ID NO: 101 - DNA, 835 bp, Penicillium sp.
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 102 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 103 - DNA, 977 bp, Thielavia terrestris
atgaagctgtcatcccagctcgccgccctcacgctggccgcggcctccgtgtcaggccactacatcttcgagcagattgcccatggcggcaccaagttcccaccttacgagtacatccgaagaaacacgaactataacagccctgtcaccagtctctcgtcgaacgacctgcgatgcaacgtaggcggcgagacggctggcaacacgaccgtcctcgacgtgaaggcgggcgactccttcaccttctactcggacgtggccgtgtaccaccaggggcccatctcactgtgcgtgccccgggccaactttgatcagtcccaagcggactgtccgctcgcctggataaccacaattgactgacagcccgcacagctacatgtccaaggctcccggctccgtcgtggactacgacggctccggcgactggttcaagatccacgactggggcccgaccttcagcaacggccaggcctcgtggccgctgcggggtgcgtcccttccctttccctcccccttcctcccccttcctccccccctttccccccttttctgtctggtcgcacgccctgctgacgtccccgtagacaactaccagtacaacatcccgacgtgcatcccgaacggcgagtacctgctgcgcatccagtcgctggcgatccacaacccgggcgccacgccgcagttctacatcagctgcgcgcaggtccgggtctcgggcggcggcagcgcctccccctccccaacggccaagatccccggcgcgttcaaggcgaccgatcccgggtataccgcgaatgtgagtgccctatgttccttgcgctccttgttccttgctccttgctcggcgtgcttgaacgctacgggctgtggagggagggatggatggatgaataggatgctgactgatggtgggacaccagatttacaataacttccactcgtatacggtgccgggtccggcggtctttcagtgctag

SEQ ID NO: 104 - protein, 223 aa, Thielavia terrestris
MKLSSQLAALTLAAASVSGHYIFEQIAHGGTKFPPYEYIRRNTNYNSPVTSLSSNDLRCNVGGETAGNTTVLDVKAGDSFTFYSDVAVYHQGPISLYMSKAPGSVVDYDGSGDWFKIHDWGPTFSNGQASWPLRDNYQYNIPTCIPNGEYLLRIQSLAIHNPGATPQFYISCAQVRVSGGGSASPSPTAKIPGAFKATDPGYTANIYNNFHSYTVPGPAVFQC

SEQ ID NO: 105 - DNA, 878 bp, Thielavia terrestris
atgaagttctcactggtgtctctgctggcttacggcctctcggtcgaggcgcactccatcttccaggttcgtctcgcacatcacgctcaactcggctcgtggcgtaagggcaaggattaacacggccggcagagagtctcggtcaacggccaagaccaaggcctgctcaccggcctccgcgctccaagcaacaacaacccagtgcaagatgtcaacagccagaacatgatttgcggccagtcgggctccaagtcgcagaccgttatcaacgtcaaggccggcgacaggatcggctcgctctggcagcatgtcatcggcggcgcccagttttcgggtgacccggacaacccgatcgcccactcgcacaagggccccgtgatggcgtaccttgctaaggtcgacaatgccgcgtccgcgagccaaacgggtctgaagtggtaagtagcgggcgacgctcaggggacggggatcgggggcctgctccatccgagactaacaccgtggacaggttcaagatctggcaggacgggttcgataccagcagcaagacatggggcgtcgacaacctgatcaagaacaacggctgggtgtacttccacctgccgcagtgcctcgctccgggccagtatctcctgcgcgtcgaggttctggcgctgcactcggcgtaccagcagggccaggcccagttctaccagtcctgcgcccagatcaacgtctccggctccgggtccttcagcccgtcccagacggtcagcatcccgggcgtctacagcgccaccgacccgagcatcctcatcaacatctacggcagcacggggcagcccgacaacggcggcaaggcttacaacccccctggacccgccccgatctcctgctga

SEQ ID NO: 106 - protein, 246 aa, Thielavia terrestris
MKFSLVSLLAYGLSVEAHSIFQRVSVNGQDQGLLTGLRAPSNNNPVQDVNSQNMICGQSGSKSQTVINVKAGDRIGSLWQHVIGGAQFSGDPDNPIAHSHKGPVMAYLAKVDNAASASQTGLKWFKIWQDGFDTSSKTWGVDNLIKNNGWVYFHLPQCLAPGQYLLRVEVLALHSAYQQGQAQFYQSCAQINVSGSGSFSPSQTVSIPGVYSATDPSILINIYGSTGQPDNGGKAYNPPGPAPISC

SEQ ID NO: 107 - DNA, 1253 bp, Thielavia terrestris
atgaggacgacattcgccgccgcgttggcagccttcgctgcgcaggaagtggcaggccatgccatcttccaacagctctgggtggacggcaccgactatatacgtgctccccttttccttttgtgtttgcccatcctcgattgataacccgaggccatccaatgctgactcttacagcacggctcctcctgcgtccgcatgccgctgtcgaactcgcccgtcacgaacgtcggcagcagggacatgatctgcaacgccggcacgcgccccgtcagcgggaagtgccccgtcaaggccggcggcaccgtgacggttgagatgcaccaggtgggctgatttcctgagcgtcctattcctcccggaagcccctttcccatcctttgccctggctaacccctccgcccctcccagcaacccggggatcggtcgtgtaacaacgaagccatcggcggcgcccactggggaccggtgcaggtgtacctcagcaaggtggaggacgcgagcacggcggacgggtcgacgggctggttcaagatcttcgcggacacgtggtccaagaaggcgggcagctcggtgggggacgacgacaactggggcacgcgcgacctcaacgcgtgctgcggcaagatgcaggtcaagatcccggcggacatcccgtcgggcgactacctgctgcgggcggaggcgctggcgctgcacacggcgggccaggtgggcggcgcgcagttctacatgagctgctaccagatcaccgtgtcgggcggcggcagcgccagcccggccaccgtcaagttccccggcgcctacagcgccaacgacccgggcatccacatcaacatccacgcggccgtgtccaactacgtcgcgcccggcccggccgtctattccggcggcacgaccaaggtggccgggtccgggtgccaaggctgcgagaacacgtgcaaggtcggctcgtcgcccacggcgacggcgccgtcgggcaagagcggcgcgggttccgacggcggcgctgggaccgacggcgggtcttcgtcttcgagccccgacacgggcagcgcgtgcagcgtgcaggcctacgggcagtgcggcgggaacgggtactcgggttgcacccagtgcgcggtaagttcggggtcgtctgtcttttgtaggaacatccgagaggcttggctgacgaggcgttgttgtagcccggctatacttgcaaggcggtctctccgccgtactattcgcagtgcgccccttcttcttag

SEQ ID NO: 108 - protein, 334 aa, Thielavia terrestris
MRTTFAAALAAFAAQEVAGHAIFQQLWHGSSCVRMPLSNSPVTNVGSRDMICNAGTRPVSGKCPVKAGGTVTVEMHQQPGDRSCNNEAIGGAHWGPVQVYLSKVEDASTADGSTGWFKIFADTWSKKAGSSVGDDDNWGTRDLNACCGKMQVKIPADIPSGDYLLRAEALALHTAGQVGGAQFYMSCYQITVSGGGSASPATVKFPGAYSANDPGIHINIHAAVSNYVAPGPAVYSGGTTKVAGSGCQGCENTCKVGSSPTATAPSGKSGAGSDGGAGTDGGSSSSSPDTGSACSVQAYGQCGGNGYSGCTQCAPGYTCKAVSPPYYSQCAPSS

SEQ ID NO: 109 - DNA, 798 bp, Thielavia terrestris
atgaagctgagcgttgccatcgccgtgctggcgtcggctcttgccgaggctcactgtgagtgcatcgtctcactccagctactgcgaagcttgctgacgatggtccctagacaccttccccagcatcggaaacaccgctgactggcagtatgtgcggattacaacgaactaccagagcaacgggccggtgacggacgtcacctcggatcaaattcggtgctacgaacggaacccaggcacgggagcgcagggcatatacaacgtcaccgccggccagaccatcaactacaacgcgaaggcgtccatctcccacccggggcccatgtccttctacattgctaaggttcccgccggccaaaccgctgcgacctgggacggtaagggggctgtgtggaccaagatctaccaggacatgcccaagttcggcagcagcctgacctggcccaccatgggtaagaattctcaccctggaaatgaacgcacatttgcacagatctaacatggcctacaggcgccaagtctgtccccgtcaccatccctcgttgcctccagaacggcgattaccttctgcgagccgagcacatcgctctacacagcgcgagcagcgtcggtggcgcccagttctacctctcgtgcgcccagcttactgtcagcggcggcagtggcacctggaaccccaagaaccgggtctccttccccggcgcttacaaggcaacagacccgggcatcttgatcaacatctactaccccgtgccgaccagctactcgccgcccggcccgccggctgagacgtgctaa

SEQ ID NO: 110 - protein, 227 aa, Thielavia terrestris
MKLSVAIAVLASALAEAHYTFPSIGNTADWQYVRITTNYQSNGPVTDVTSDQIRCYERNPGTGAQGIYNVTAGQTINYNAKASISHPGPMSFYIAKVPAGQTAATWDGKGAVWTKIYQDMPKFGSSLTWPTMGAKSVPVTIPRCLQNGDYLLRAEHIALHSASSVGGAQFYLSCAQLTVSGGSGTWNPKNRVSFPGAYKATDPGILINIYYPVPTSYSPPGPPAETC

SEQ ID NO: 111 - DNA, 1107 bp, Thielavia terrestris
atgccttctttcgcctccaagactctcctttccaccctggcgggtgccgcatccgtggccgcccacgggcacgtgtcgaacatcgtcatcaacggggtctcgtaccagggttacgatccgacctccttcccttacatgcagaacccgcccatcgtggtcggctggactgccgccgacacggacaacggctttgttgccccggatgccttcgccagtggcgatatcatctgccacaagaacgccaccaacgccaagggccacgccgtggtcgccgcgggagacaagatcttcatccagtggaacacatggcccgagtcccaccacggccccgtcatcgactacctcgcgagctgcggcagcgcgtcctgcgagaccgtcgacaagaccaagctcgagttcttcaagatcgacgaggtcggcctggtcgacggcagctcggcgcccggtgtgtggggctccgaccagctcatcgccaacaacaactcgtggctcgtcgagatcccgcccaccatcgcgccgggcaactacgtcctgcgccacgagatcatcgcgctgcacagcgccgaaaacgccgacggcgcccagaactacccgcagtgcttcaacctgcagatcaccggcaccggcaccgccaccccctccggcgtccccggcacctcgctctacaccccgaccgacccgggcatcctcgtcaacatctacagcgccccgatcacctacaccgtcccggggccggccctcatctccggcgccgtcagcatcgcccagtcctcctccgccatcaccgcctccggcaccgccctgaccggctctgccaccgcacccgccgccgccgctgctaccacaacttccaccaccaacgccgcggctgctgctacctctgctgctgctgctgctggtacttccacaaccaccaccagcgccgcggccgtggtccagacctcctcctcctcctcctccgccccgtcctctgccgccgccgccgccaccaccaccgcggctgccagcgcccgcccgaccggctgctcctctggccgctccaggaagcagccgcgccgccacgcgcgggatatggtggttgcgcgaggggctgaggaggcaaactga

SEQ ID NO: 112 - protein, 368 aa, Thielavia terrestris
MPSFASKTLLSTLAGAASVAAHGHVSNIVINGVSYQGYDPTSFPYMQNPPIVVGWTAADTDNGFVAPDAFASGDIICHKNATNAKGHAVVAAGDKIFIQWNTWPESHHGPVIDYLASCGSASCETVDKTKLEFFKIDEVGLVDGSSAPGVWGSDQLIANNNSWLVEIPPTIAPGNYVLRHEIIALHSAENADGAQNYPQCFNLQITGTGTATPSGVPGTSLYTPTDPGILVNIYSAPITYTVPGPALISGAVSIAQSSSAITASGTALTGSATAPAAAAATTTSTTNAAAAATSAAAAAGTSTTTTSAAAVVQTSSSSSSAPSSAAAAATTTAAASARPTGCSSGRSRKQPRRHARDMVVARGAEEAN

SEQ ID NO: 113 - DNA, 993 bp, Thielavia terrestris
atgccgcccgcactccctcaactcctaaccacggtcctgaccgccctcaccctcggttccaccgccctcgcccactcacacctcgcgtacattatcgttaacggcaagctctaccagggcttcgacccgcgcccgcaccaggccaactacccttcccgggtcgggtggtccaccggcgccgtcgacgacggcttcgtcacgccggccaactactccaccccggacatcatttgccacatcgccggcaccagcccggccggccacgcgcccgtgcgcccgggcgaccgcatccacgtccagtggaacggctggccggtcggccacatcggtcccgtgctgtcgtacctcgcccgctgcgagtcggacacgggctgcacgggccagaacaagaccgcgctgcggtggaccaagatcgacgactccagcccgaccatgcagaacgtcgccggcgcgggcacccagggcgagggcacccccggcaagcgctgggccaccgacgtgctgatcgccgccaacaacagctggcaggtcgccgtgccggcggggctgccgaccggcgcgtacgtgctgcgcaacgagatcatcgcgctgcactacgcggcgaggaagaacggggcgcagaactatccgctctgcatgaacctgtgggtggacgccagtggtgataatagtagtgtggctgcaacgacggcggcggtgacggcggggggtctgcagatggatgcgtatgacgcgcgcgggttctacaaggagaacgatccgggcgtgctggtcaatgtcacggccgcgctgtcgtcgtatgtcgtgcccgggccgacggtggcggcgggcgccacgccggtgccgtacgcgcagcagagcccgagcgtgtcgacggcggcgggcacgcccgtcgtcgttacaaggactagcgagacggcgccgtacacgggcgccatgacgccgacggttgcggcgaggatgaaggggagggggtatgatcggcggggttag

SEQ ID NO: 114 - protein, 330 aa, Thielavia terrestris
MPPALPQLLTTVLTALTLGSTALAHSHLAYIIVNGKLYQGFDPRPHQANYPSRVGWSTGAVDDGFVTPANYSTPDIICHIAGTSPAGHAPVRPGDRIHVQWNGWPVGHIGPVLSYLARCESDTGCTGQNKTALRWTKIDDSSPTMQNVAGAGTQGEGTPGKRWATDVLIAANNSWQVAVPAGLPTGAYVLRNEIIALHYAARKNGAQNYPLCMNLWVDASGDNSSVAATTAAVTAGGLQMDAYDARGFYKENDPGVLVNVTAALSSYVVPGPTVAAGATPVPYAQQSPSVSTAAGTPVVVTRTSETAPYTGAMTPTVAARMKGRGYDRRG

SEQ ID NO: 115 - DNA, 1221 bp, Thielavia terrestris
atgaagacattcaccgccctcctggccgcagccggcctcgtcgccggccatggatatgtcgacaacgccaccattggcggccagttttatcaggtactctaccgcttcacccaaggtccgctggccacaactctataggtgtcataaattaacaagccaccgtcccgcagttctatcaggtgtgctcgctaccgaccatgtggtcccgtctcagcaagccactcacacgcccatgatcccctagccttacgtcgacccgtatttagcaaccttggcacgtagtatttattgtcccaaatattgagctgaactgcacctccctagaatcccgcggtgctaacattctttcagcccgacagggtctctcgatccatcccgggcaacggcccggtcacggacgtcactctcatcgacctgcagtgcaacgccaattccaccccggccaagctccacgccactgccgctgccggctcggacgtgattctccgctggacgctctggcctgagtcgcacgttggccccgtcatcacctacatggcccgctgccccgacacgggctgccaggactggatgccgggcacttcgtaggagcccatcttgcaccatatccatttcaaccggccacacgcactgacccatatgtctgtctacccctgcagtgcggtctggttcaagatcaaggagggcggccgcgacggcacttccaacacctgggccgacgtacgtgtaccccgtcccagagagccaaagcccccccttcaacaaagcaaacatctcaatagcccgagcctacgcactaacccctctccttccccctcgaaaacacagaccccgctgatgacggcgcccacctcgtacacgtacacgatcccctcctgcctgaagaagggctactacctggtccgccacgagatcatcgcgctgcacgccgcctacacctaccccggcgcgcagttctacccgggctgccaccagctcaacgtcacgggcggcgggtccaccgtaccgtcgagcggcctggtggcctttcccggggcgtacaagggcagtgaccccgggattacgtacgatgcgtataaaggtgggttggctggttggcccaggtcttggtgatgggggaatgtggtgatgaggtttattatttgggatcccgtggctaacgtaaccctgggtgtagcgcaaacgtaccagattcctgggccggcggtctttacttgctga

SEQ ID NO: 116 - protein, 236 aa, Thielavia terrestris
MKTFTALLAAAGLVAGHGYVDNATIGGQFYQNPAVLTFFQPDRVSRSIPGNGPVTDVTLIDLQCNANSTPAKLHATAAAGSDVILRWTLWPESHVGPVITYMARCPDTGCQDWMPGTSAVWFKIKEGGRDGTSNTWADTPLMTAPTSYTYTIPSCLKKGYYLVRHEIIALHAAYTYPGAQFYPGCHQLNVTGGGSTVPSSGLVAFPGAYKGSDPGITYDAYKAQTYQIPGPAVFTC

SEQ ID NO: 117 - DNA, 933 bp, Thielavia terrestris
atggccttgctgctcttggcaggcttggccattctggccgggccggctcatgcccacggcggcctcgccaactacacagtgggcaacacctggtatagggggtgcgtaaggggggcaccgacaacgcctgcttagtaactccaccatttcgagcgggctaacaccgggcgcagctacgaccccttcacgccggcggccgaccagatcggccagccgtggatgatccaacgcgcgtgggactcgatcgacccgatcttcagcgtcaacgacaaggcgctcgcctgcaacaccccggccacggcgccgacctcttacattcccatccgcgcgggcgagaacatcacggccgtgtactggtactggctgcacccggtgggccccatgacggcgtggctggcgcggtgcgacggcgactgccgcgacgccgacgtcaacgaggcgcgctggttcaagatctgggaggccggcctgctcagcgggccgaacctggccgagggcatgtggtaccagaaggcgttccagaactgggacggcagcccggacctgtggcccgtcacgatcccggccgggctgaagagcggcctgtacatgatccggcacgagatcttgtcgatccacgtcgaggataaaccgcagttttatcccgagtgtgcgcatctgaatgtgaccgggggtggggacctgctgccgcctgatgagtttttggtgaagttcccgggcgcttacaaagaagatagtgagtgaaacgcgaagcttcggtagccattgggttgcgctgatggaggttagacccgtcgatcaagatcaatatctactcggaccagtacgccaatacaacggtgagtgtaacaggtcgagcaaaaccaaacagatgccgatgactgatgatctcagaattacacaattcccggagggccgatatgggatgggtga

SEQ ID NO: 118 - protein, 250 aa, Thielavia terrestris
MALLLLAGLAILAGPAHAHGGLANYTVGNTWYRGYDPFTPAADQIGQPWMIQRAWDSIDPIFSVNDKALACNTPATAPTSYIPIRAGENITAVYWYWLHPVGPMTAWLARCDGDCRDADVNEARWFKIWEAGLLSGPNLAEGMWYQKAFQNWDGSPDLWPVTIPAGLKSGLYMIRHEILSIHVEDKPQFYPECAHLNVTGGGDLLPPDEFLVKFPGAYKEDNPSIKINIYSDQYANTTNYTIPGGPIWDG

SEQ ID NO: 119 - DNA, 1584 bp, Thielavia terrestris
atgatgccgtcccttgttcgcttctcaatgggtctggcgaccgccttcgcctcgctgtccacagcacataccgtcttcaccacgcttttcatcaacggcgtcgaccaaggggacgggacctgcatccgcatggccaagaagggcagcgtttgcacccatcccattgctggtggcctcgacagcccagacatggcttgtggtatgccctctgcgtttcccctgcgagagctttcctcgagctaacccaatgccgcgttgcccaggccgagacggacaacaagccgtggcattcacctgcccagccccggcgggctccaagttgagcttcgagttccgcatgtgggccgacgcctctcagcccggctctatcgacccatcccacctcggctcgacggcaatctacctcaaacaagtctccaacatcagctccgactcggctgccggccctggctggttcaagatctacgccgagggctacgacacagccgccaagaagtgggccacagagaagctcatcgacaacggcggcctgctgagcatcgagcttccgcccactctgccggcgggatactacctcgcccgcagcgagatcgtcaccatccagaacgtcaccaacgaccacgtcgacccgcagttctacgttggctgcgcacagctcttcgtccaggggcctccgaccacccccaccgtcccgccagacagactcgtctccatcccgggccacgtccatgcctccgacccggggctgaccttcaacatctggcgcgacgacccctccaagacggcctacaccgtcgtcggcccggcccccttctcccccaccgccgcccccacccccacctccaccaacaccaacgggcagcaacaacaacaacagcaacaggcgataaagcagacggacggcgtgatccccgccgactgccagctcaagaacgccaactggtgcggcgccgaggtgcccgcgtacgccgacgaggccggctgctgggcgtcgtcggccgactgcttcgcccagctggacgcctgctacacgtcggcgccgcccacgggcagccgcggctgccggctgtgggaggactggtgcaccggcattcagcagggctgccgcgcggggcggtggcgggggccgccgccctttcatggggagggggcagcagcggaggtgtgaacggttcggggacgggtggcggtggtggtggtggtggtggtggcactggctcttcttcggcttctgccccgacggagacggcctctgctggccgggggggcgcaagaatagctgccgtggccggctgcggaggcgggacaggagacatggttgaagaggttttcctcttttattgggacgcttgcagcggctggcgacggagccgtggtggtggttcgattcttgcgaggcttatccttcatgtccttcttccacttttgagaccgaggcgagcccctcgagtccatttacttctcttccacctgtacctcaacttctgttatccaggaaccagtggtttctataatcgcctgagcattaaactaggcatatggccaagcaaaatgtcgcctgatgtagcgcattacgtgaaataa

SEQ ID NO: 120 - protein, 478 aa, Thielavia terrestris
MMPSLVRFSMGLATAFASLSTAHTVFTTLFINGVDQGDGTCIRMAKKGSVCTHPIAGGLDSPDMACGRDGQQAVAFTCPAPAGSKLSFEFRMWADASQPGSIDPSHLGSTAIYLKQVSNISSDSAAGPGWFKIYAEGYDTAAKKWATEKLIDNGGLLSIELPPTLPAGYYLARSEIVTIQNVTNDHVDPQFYVGCAQLFVQGPPTTPTVPPDRLVSIPGHVHASDPGLTFNIWRDDPSKTAYTVVGPAPFSPTAAPTPTSTNTNGQQQQQQQQAIKQTDGVIPADCQLKNANWCGAEVPAYADEAGCWASSADCFAQLDACYTSAPPTGSRGCRLWEDWCTGIQQGCRAGRWRGPPPFHGEGAAAETASAGRGGARIAAVAGCGGGTGDMVEEVFLFYWDACSGWRRSRGGGSILARLILHVLLPLLRPRRAPRVHLLLFHLYLNFCYPGTSGFYNRLSIKLGIWPSKMSPDVAHYVK

SEQ ID NO: 121 - DNA, 868 bp, Thielavia terrestris
atgcagctcctcgtgggcttgctgcttgcagccgtggctgctcgagcacattgtatttctacccctttccgcgtgcctcccagcctcaaggcaagaagacgcacgcagcagctaacggaccctatcagacacatttcccagactcgtggtaaatgggcagcccgaggacaaggactggtcggttacgcgcatgaccaagaacgcgcagagcaagcagggagtccaggacccgaccagtcccgacattcgctgctacacgtcgcagacggcgcctaacgtggctacggtccctgccggagccaccgtccattacatatcgactcagcagatcaaccacccgggcccgacgcagtactacctcgccaaggtaccggcggggtcgtcggccaagacgtgggacgggtcaggggccgtctggttcaagatctcgaccaccatgccttacttggacaacaacaagcagcttgtctggccgaatcagagtaggaacaattcccgctccaatcttcgatttggccttgagctacggccgattgcatgggagagaccgttgactgacggggcaacccaaccttcatcagacacgtacacgacggtcaacacgaccatccccgccgatacgcccagtggggaatacctcctccgggtcgagcagatcgcgctgcacctggcctcgcagcccaacggggctcagttctacctggcctgctcgcagatccagattacgggcggcggcaacggcacgcccggcccgctagtcgcgttgccgggggcgtacaagagcaacgacccgggcattttggtcaacatctactctatgcagcccggcgattacaagccgcccgggccgccggtgtggagtggctga

SEQ ID NO: 122 - protein, 230 aa, Thielavia terrestris
MQLLVGLLLAAVAARAHYTFPRLVVNGQPEDKDWSVTRMTKNAQSKQGVQDPTSPDIRCYTSQTAPNVATVPAGATVHYISTQQINHPGPTQYYLAKVPAGSSAKTWDGSGAVWFKISTTMPYLDNNKQLVWPNQNTYTTVNTTIPADTPSGEYLLRVEQIALHLASQPNGAQFYLACSQIQITGGGNGTPGPLVALPGAYKSNDPGILVNIYSMQPGDYKPPGPPVWSG

SEQ ID NO: 123 - DNA, 1068 bp, Thielavia terrestris
atgaagctgtacctggcggcctttctaggcgccgtcgccaccccgggagcgttcgctcatcgtaggttccccgtctatctccctaggggtagcaccacgactaatttctcgtcgtccccctgtagaaatccacgggattctacttgtcaacggcaccgaaacgccggaatggaaatacgtccggtaatatctaccttgctctccttcttccacaaccagcctaacacatcatcagtgacgtggcctgggagggcgcctacgaaccggaaaaataccccaacaccgagttctttaagacgcccccgcagacggacatcaacaacccgaacatcacctgcggcaggaacgcgttcgactcggccagcaagactgagacggccgacatactggccggctcagaggtcggcttccgcgtctcgtgggacggcaacggcaagtacggcgtgttctggcatcccgggccggggcagatctacctctctcgtgctccgaacgacgacctggaggactaccgcggcgacggagactggttcaagatcgcaaccggcgccgccgtctccaataccgagtggctgctgtggaacaagcatgacgtgagccccaacattcctcgcccaatcgatccccaacctggtcaccatggcggcgtccgggatgcaaagagactaactccagaggaacctacctagttcaacttcaccatccccaagacgacgccgccgggcaagtacctgatgcgcatcgagcagttcatgccctccacggtcgaatacagccagtggtacgtcaactgcgcccacgtcaacatcatcggccccggcggaggcacgccgacgggctttgccaggtttcccggcacctacactgttgacgatcccggtaagccggacctaccggacacagaggcctcgggatagcttgctaaccttgtttgctctctctctttttctctcccgactaggcatcaaggtgccgttgaaccagatcgtcaacagcggagagttgccgcaggaccaactgaggctgctcgagtacaagcccccgggcccagcgctgtggactggttga

SEQ ID NO: 124 - protein, 257 aa, Thielavia terrestris
MKLYLAAFLGAVATPGAFAHQIHGILLVNGTETPEWKYVRDVAWEGAYEPEKYPNTEFFKTPPQTDINNPNITCGRNAFDSASKTETADILAGSEVGFRVSWDGNGKYGVFWHPGPGQIYLSRAPNDDLEDYRGDGDWFKIATGAAVSNTEWLLWNKHDFNFTIPKTTPPGKYLMRIEQFMPSTVEYSQWYVNCAHVNIIGPGGGTPTGFARFPGTYTVDDPGIKVPLNQIVNSGELPQDQLRLLEYKPPGPALWTG

SEQ ID NO: 125 - DNA, 871 bp, Thermoascus crustaceus
atggccttttcccagataatggctattaccggcgtttttcttgcctctgcttccctggtggctggccatggctttgttcagaatatcgtgattgatggtaaaaggtacctaactacctaccttactatctgatgtcatttacaagaaagggcacagacacaagcggcaaaaaaaagaaagaaagaaagaaagaaagaaagctgacaaaaattcaacaagttatggcgggtacatcgtgaaccaatatccatacatgtcagatcctccggaggtcgtcggctggtctaccaccgcaaccgacctcggattcgtggacggtaccggataccaaggacctgatatcatctgccacaggggcgccaagcctgcagccctgactgcccaagtggccgccggaggaaccgtcaagctggaatggactccatggcctgattctcaccacggcccggtgatcaactaccttgctccttgcaacggtgactgttccaccgtggacaagacccaattgaaattcttcaagatcgcccaggccggtctcatcgatgacaacagtcctcctggtatctgggcctcagacaatctgatagcggccaacaacagctggactgtcaccatcccaaccacaactgcacctggaaactatgttctaaggcatgagatcattgctctccactcagctgggaacaaggatggtgcgcagaactatccccagtgcatcaacctgaaggtcactggaaatggttctggcaatcctcctgctggtgctcttggaacggcactctacaaggatacagatccgggaattctgatcaatatctaccagaaactttccagctatgttattcctggtcctgctttgtacactggttag

SEQ ID NO: 126 - protein, 251 aa, Thermoascus crustaceus
MAFSQIMAITGVFLASASLVAGHGFVQNIVIDGKSYGGYIVNQYPYMSDPPEVVGWSTTATDLGFVDGTGYQGPDIICHRGAKPAALTAQVAAGGTVKLEWTPWPDSHHGPVINYLAPCNGDCSTVDKTQLKFFKIAQAGLIDDNSPPGIWASDNLIAANNSWTVTIPTTTAPGNYVLRHEIIALHSAGNKDGAQNYPQCINLKVTGNGSGNPPAGALGTALYKDTDPGILINIYQKLSSYVIPGPALYTG

SEQ ID NO: 127 - DNA, 1102 bp, Thermoascus crustaceus
atgtcattctcgaagatacttgctatcgctggggccattacctacgcatcttcagctgccgctcatggttatgtccagggaattgttgtcgatggcagctagtatgtcactctggatggaaccttcagcacgtactgtactaacaatcagcagctacgggggatatatggtgacccaatatccctacaccgctcaacctccggaactcatcgcctggtccactaaagcaaccgatcttgggtttgtggacggcagtggctatacttctcctgatatcatctgccataagggtgctgagcctggtgcccagagcgccaaagtggcagctggagggaccgttgagctgcagtggacggcatggcccgagtctcacaagggcccagttattgactacctcgccgcctgcgacggggactgctcatctgttgataagactgcactaaagttctttaagattgacgagagtggtctgattgacggcaacggtgctggaacatgggcctctgatacgttgatcaaaaataacaacagctggactgtcaccatcccaagcacaattgcttccggaaactacgtactaagacacgaaataattgcgctccattctgccggaaacaaagatggtgctcagaactatccccagtgtatcaacctcgaggtcactggtagtggcaccgaaaaccctgctggcactctcggaacagcgctttacacagacactgatcctggccttctggtcaacatctaccagggtctgtccaactattcaatccctggtcctgctctgtatagcggcaacagtgataacgctggttccctcaaccctaccaccacgccgtcaattcagaatgctgctgctgctccctccacttccacagcatctgttgtcactgattcttcgtcagccacccagactgctagtgtcgccgccacgactccagcctccacttcggctgttacagcctcaccagctcccgatactggaagcgacgtaaccaaatatctggattcgatgagctcggatgaggtcctcaccctggtgcgcgggaccctgtcttggctggtttctaacaagaaacatgcgcgggatctttctcactga

SEQ ID NO: 128 - protein, 349 aa, Thermoascus crustaceus
MSFSKILAIAGAITYASSAAAHGYVQGIVVDGSYYGGYMVTQYPYTAQPPELIAWSTKATDLGFVDGSGYTSPDIICHKGAEPGAQSAKVAAGGTVELQWTAWPESHKGPVIDYLAACDGDCSSVDKTALKFFKIDESGLIDGNGAGTWASDTLIKNNNSWTVTIPSTIASGNYVLRHEIIALHSAGNKDGAQNYPQCINLEVTGSGTENPAGTLGTALYTDTDPGLLVNIYQGLSNYSIPGPALYSGNSDNAGSLNPTTTPSIQNAAAAPSTSTASVVTDSSSATQTASVAATTPASTSAVTASPAPDTGSDVTKYLDSMSSDEVLTLVRGTLSWLVSNKKHARDLSH

SEQ ID NO: 129 - DNA, 1493 bp, Thermoascus crustaceus
atgttgtcattcattcccaccaagtcagctgcgctgacgactcttctacttcttggaacagctcatgctcacactttgatgaccaccatgtttgtggacggcgtcaaccagggagatggtgtctgcattcgcatgaacaatgacggcggaactgccaatacctatatccagcctatcacgagcaaggatatcgcctgcggtaagtacccagatgtcatcatactctgccataacatccgtcatatctactagaatcggagcaatgttaagtatttccaggcatccaaggcgaaatcggcgcctcccgagtctgcccagtcaaggcatcttccaccctaaccttccaattccgcgagcaacccaacaacccaaactcctcccctctcgatccatcgcacaaaggccccgccgcggtgtacctgaaaaaggtcgactccgccatcgcgagcaacaacgccgccggagacagctggttcaagatctgggagtccgtctacgacgagtccacgggcaaatggggcacgaccaagatgatcgagaacaacgggcacatctccgtcaaggtgcccgatgatatcgagggtggttactatcttgcccggacggagctgctggcgctacattctgcggatcagggggatccgcagttctatgttggctgtgcgcagctgtttatcgattcggatgggacggcgaaaccgcccactgtttctattggagaggggacgtacgatctgagcatgcctgccatgacgtataatatctgggagacaccgttggctctgccgtatccgatgtatgggcctcctgtctatacgcctggctctggttctggatcagtccgtgcgacgagctcttctgctgtccctactgcaaccgaatcctcttttgtagaggaaagagcaaaccccgtcacggcaaacagtgtttattctgcaaggggcaaattcaaaacctggattgataaactgtcatggcgcgggaaggtccgtgagaacgtcagacaagccgcgggaagaagaagcactctcgtccagactgtgggtctaaagccaaaaggctgcatcttcgtcaatggaaactggtgcggcttcgaggttcccgactacaacgatgcggagagctgctgggctgtatgttcccctccttagcctcttacatccctaagtactacatttgaaaacaacaaaaagaaatgtatatactaactacgtacgctctactctaggcctccgacaactgctggaaacagtccgacgcctgctggaacaagacccaacccacgggctacaataactgccagatctggcaggacaagaaatgcaaggtcatccaggattcctgtagcggacccaacccgcatggaccaccgaataagggcaaggatttgactccggagtggccgccactgaagggctcgatggatacgttctccaagcgtactatcggttaccgcgattggattgttagaaggagaggtgcatgagggtgta

SEQ ID NO: 130 - protein, 436 aa, Thermoascus crustaceus
MLSFIPTKSAALTTLLLLGTAHAHTLMTTMFVDGVNQGDGVCIRMNNDGGTANTYIQPITSKDIACGIQGEIGASRVCPVKASSTLTFQFREQPNNPNSSPLDPSHKGPAAVYLKKVDSAIASNNAAGDSWFKIWESVYDESTGKWGTTKMIENNGHISVKVPDDIEGGYYLARTELLALHSADQGDPQFYVGCAQLFIDSDGTAKPPTVSIGEGTYDLSMPAMTYNIWETPLALPYPMYGPPVYTPGSGSGSVRATSSSAVPTATESSFVEERANPVTANSVYSARGKFKTWIDKLSWRGKVRENVRQAAGRRSTLVQTVGLKPKGCIFVNGNWCGFEVPDYNDAESCWAASDNCWKQSDACWNKTQPTGYNNCQIWQDKKCKVIQDSCSGPNPHGPPNKGKDLTPEWPPLKGSMDTFSKRTIGYRDWIVRRRGA

SEQ ID NO: 131 - DNA, 1415 bp, Aspergillus fumigatus
atggtccatctatcttcattggcagcagccctggctgctctgcctctgtatgtttacccactcacgagaggaggaacagctttgacattgctatagtgtatatggagctggcctgaacacagcagccaaagccaaaggactaaagtactttggttccgccacggacaatccagagctcacggactctgcgtatgtcgcgcaactgagcaacaccgatgattttggtcaaatcacacccggaaactccatgaaggtttgcttacgtctgcctccctggagcattgcctcaaaagctaattggttgttttgtttggatagtgggatgccaccgagccttctcagaattctttttcgttcgcaaatggagacgccgtggtcaatctggcgaacaagaatggccagctgatgcgatgccatactctggtctggcacagtcagctaccgaactggggtatgtaaacgtcttgtctattctcaaatactctctaacagttgacagtctctagcgggtcatggaccaatgcgacccttttggcggccatgaagaatcatatcaccaatgtggttactcactacaaggggaagtgctacgcctgggatgttgtcaatgaaggtttgttgctccatctatcctcaatagttcttttgaaactgacaagcctgtcaatctagccctgaacgaggacggtactttccgtaactctgtcttctaccagatcatcggcccagcatacattcctattgcgttcgccacggctgctgccgcagatcccgacgtgaaactctactacaacgactacaacattgaatactcaggcgccaaagcgactgctgcgcagaatatcgtcaagatgatcaaggcctacggcgcgaagatcgacggcgtcggcctccaggcacactttatcgtcggcagcactccgagtcaatcggatctgacgaccgtcttgaagggctacactgctctcggcgttgaggtggcctataccgaacttgacatccgcatgcagctgccctcgaccgccgcaaagctggcccagcagtccactgacttccaaggcgtggccgcagcatgcgttagcaccactggctgcgtgggtgtcactatctgggactggaccgacaagtactcctgggtccccagcgtgttccaaggctacggcgccccattgccttgggatgagaactatgtgaagaagccagcgtacgatggcctgatggcgggtcttggagcaagcggctccggcaccacaacgaccactactactacttctactacgacaggaggtacggaccctactggagtcgctcagaaatggggacagtgtggcggtattggctggaccgggccaacaacttgtgtcagtggtaccacttgccaaaagctgaatgactggtactcacagtgcctgtaa

SEQ ID NO: 132 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 133 - DNA, 2564 bp, Trichoderma reesei
ggacagccggacgcaatggtgaataacgcagctcttctcgccgccctgtcggctctcctgcccacggccctggcgcagaacaatcaaacatacgccaactactctgctcagggccagcctgatctctaccccgagacacttgccacgctcacactctcgttccccgactgcgaacatggccccctcaagaacaatctcgtctgtgactcatcggccggctatgtagagcgagcccaggccctcatctcgctcttcaccctcgaggagctcattctcaacacgcaaaactcgggccccggcgtgcctcgcctgggtcttccgaactaccaagtctggaatgaggctctgcacggcttggaccgcgccaacttcgccaccaagggcggccagttcgaatgggcgacctcgttccccatgcccatcctcactacggcggccctcaaccgcacattgatccaccagattgccgacatcatctcgacccaagctcgagcattcagcaacagcggccgttacggtctcgacgtctatgcgccaaacgtcaatggcttccgaagccccctctggggccgtggccaggagacgcccggcgaagacgcctttttcctcagctccgcctatacttacgagtacatcacgggcatccagggtggcgtcgaccctgagcacctcaaggttgccgccacggtgaagcactttgccggatacgacctcgagaactggaacaaccagtcccgtctcggtttcgacgccatcataactcagcaggacctctccgaatactacactccccagttcctcgctgcggcccgttatgcaaagtcacgcagcttgatgtgcgcatacaactccgtcaacggcgtgcccagctgtgccaacagcttcttcctgcagacgcttttgcgcgagagctggggcttccccgaatggggatacgtctcgtccgattgcgatgccgtctacaacgttttcaaccctcatgactacgccagcaaccagtcgtcagccgccgccagctcactgcgagccggcaccgatatcgactgcggtcagacttacccgtggcacctcaacgagtcctttgtggccggcgaagtctcccgcggcgagatcgagcggtccgtcacccgtctgtacgccaacctcgtccgtctcggatacttcgacaagaagaaccagtaccgctcgctcggttggaaggatgtcgtcaagactgatgcctggaacatctcgtacgaggctgctgttgagggcatcgtcctgctcaagaacgatggcactctccctctgtccaagaaggtgcgcagcattgctctgatcggaccatgggccaatgccacaacccaaatgcaaggcaactactatggccctgccccatacctcatcagccctctggaagctgctaagaaggccggctatcacgtcaactttgaactcggcacagagatcgccggcaacagcaccactggctttgccaaggccattgctgccgccaagaagtcggatgccatcatctacctcggtggaattgacaacaccattgaacaggagggcgctgaccgcacggacattgcttggcccggtaatcagctggatctcatcaagcagctcagcgaggtcggcaaaccccttgtcgtcctgcaaatgggcggtggtcaggtagactcatcctcgctcaagagcaacaagaaggtcaactccctcgtctggggcggatatcccggccagtcgggaggcgttgccctcttcgacattctctctggcaagcgtgctcctgccggccgactggtcaccactcagtacccggctgagtatgttcaccaattcccccagaatgacatgaacctccgacccgatggaaagtcaaaccctggacagacttacatctggtacaccggcaaacccgtctacgagtttggcagtggtctcttctacaccaccttcaaggagactctcgccagccaccccaagagcctcaagttcaacacctcatcgatcctctctgctcctcaccccggatacacttacagcgagcagattcccgtcttcaccttcgaggccaacatcaagaactcgggcaagacggagtccccatatacggccatgctgtttgttcgcacaagcaacgctggcccagccccgtacccgaacaagtggctcgtcggattcgaccgacttgccgacatcaagcctggtcactcttccaagctcagcatccccatccctgtcagtgctctcgcccgtgttgattctcacggaaaccggattgtataccccggcaagtatgagctagccttgaacaccgacgagtctgtgaagcttgagtttgagttggtgggagaagaggtaacgattgagaactggccgttggaggagcaacagatcaaggatgctacacctgacgcataagggttttaatgatgttgttatgacaaacgggtagagtagttaatgatggaataggaagaggccatagttttctgtttgcaaaccatttttgccattgcgaaaaaaaaaaaaaaaaaaaaaaaaaaaaaaaaaaaaaaaaaaaaaaaaaaaaaaaa

SEQ ID NO: 134 - protein, 780 aa, Trichoderma reesei
MVNNAALLAALSALLPTALAQNNQTYANYSAQGQPDLYPETLATLTLSFPDCEHGPLKNNLVCDSSAGYVERAQALISLFTLEELILNTQNSGPGVPRLGLPNYQVWNEALHGLDRANFATKGGQFEWATSFPMPILTTAALNRTLIHQIADIISTQARAFSNSGRYGLDVYAPNVNGFRSPLWGRGQETPGEDAFFLSSAYTYEYITGIQGGVDPEHLKVAATVKHFAGYDLENWNNQSRLGFDAIITQQDLSEYYTPQFLAAARYAKSRSLMCAYNSVNGVPSCANSFFLQTLLRESWGFPEWGYVSSDCDAVYNVFNPHDYASNQSSAAASSLRAGTDIDCGQTYPWHLNESFVAGEVSRGEIERSVTRLYANLVRLGYFDKKNQYRSLGWKDVVKTDAWNISYEAAVEGIVLLKNDGTLPLSKKVRSIALIGPWANATTQMQGNYYGPAPYLISPLEAAKKAGYHVNFELGTEIAGNSTTGFAKAIAAAKKSDAIIYLGGIDNTIEQEGADRTDIAWPGNQLDLIKQLSEVGKPLVVLQMGGGQVDSSSLKSNKKVNSLVWGGYPGQSGGVALFDILSGKRAPAGRLVTTQYPAEYVHQFPQNDMNLRPDGKSNPGQTYIWYTGKPVYEFGSGLFYTTFKETLASHPKSLKFNTSSILSAPHPGYTYSEQIPVFTFEANIKNSGKTESPYTAMLFVRTSNAGPAPYPNKWLVGFDRLADIKPGHSSKLSIPIPVSALARVDSHGNRIVYPGKYELALNTDESVKLEFELVGEEVTI

SEQ ID NO: 135 - DNA, 26 bp, Aspergillus aculeatus
  misc_feature (18)..(18): N=A,C,G, OR T
gagcagtctcggtcgggnadrttrta

SEQ ID NO: 136 - DNA, 24 bp, Aspergillus aculeatus
  misc_feature (16)..(16): N=A,C,G, OR T
gccgtcggactcgccnccnggytt

SEQ ID NO: 137 - DNA, 20 bp, Aspergillus aculeatus
ctcctacacccagggcaaca

SEQ ID NO: 138 - DNA, 20 bp, Aspergillus aculeatus
cgattggtgcaacgtcatca

SEQ ID NO: 139 - DNA, 36 bp, Aspergillus aculeatus
taagaattcaccatgcgttatacattgtctctcgca

SEQ ID NO: 140 - DNA, 32 bp, Aspergillus aculeatus
  misc_feature (12)..(12): Y=C OR T
tatgcggccgcytaraangcnggrttngcrtt

SEQ ID NO: 141 - DNA, 1410 bp, Aspergillus aculeatus
atgcgttatacattgtctctcgcagcagcgctgctgccatgcgcaatccaggcccagcaaaccctgtacggacaatgtggtggtcaaggctattccggactcaccagctgcgtggcaggagcaacatgctctaccgtgaatgaatactacgctcagtgtacgccagcagcgggcgccacctctaccaccttgaagacaactactaccactgccggggcgaccacgacgactactaccaagagctctgcttctcagacatctactactaagacctctaccggcaccgtctcgacgaccacggcgactaccacggccagcgcgagcggcaacccgttcagtgggtaccagctctacgtgaacccatactactcctccgaagtggcgtcgctggccattccatctctcactgggacgctttcctcgctccaggcagcggccacggccgcggccaaggtgccttcttttgtctggctggatgtggccgccaaggtgccgacgatggccacctacctggccgacatcaaagcccagaatgccgctggagccaatcccccgatcgcaggccaatttgtggtgtacgacctccctgaccgtgactgcgccgctctagccagtaacggcgagtactccattgccaacaacggtgtggccaactacaaggcctacatcgactccatccgcaaggtcctcgtgcagtattccgatgtgcacaccattctggtgattgagcccgacagtctcgccaacctggtgaccaacctcaacgtggccaaatgcgccaacgcccagagcgcctatctcgaatgcaccaactatgccttggagcagctgaacctccccaacgtggccatgtatctcgatgccggacacgccggctggctcggctggcccgcaaaccagcaaccggccgccaacttgtacgcgagcgtttacaagaacgctagttcccccgccgcagtgcgcggcctggccacgaatgtcgccaactacaacgccttcaccatctcctcctgcccctcctacacccagggcaacagcgtttgcgacgagcagcagtacatcaacgcgatcgccccgctcctctcagcccagggcttcgacgcccacttcatcgtcgacaccggccgcaacggcaaacagccaaccggtcagcaagcctggggcgattggtgcaacgtcatcaacaccgggttcggcgtgcgcccgaccaccagcacgggcgatgcgctcgtcgacgccttcgtctgggtgaagcccggcggcgagagcgacggcacctccgatagctcggccacccgctacgacgcccactgcgggtacagcgatgccttgcagccggcccctgaggcgggaacctggttccaggcctatttcgtgcagttgctcacgaacgctaatcccgcattctaa